SEQUENCE LISTING (51 sequences)

SEQ ID NO: 1 - protein, 22 aa, Artificial Sequence
  GMCSF signal peptide
MLLLVTSLLLCELPHPAFLLIP

SEQ ID NO: 2 - protein, 245 aa, Artificial Sequence
  CD19 heavy chain variable domain (VH)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - protein, 12 aa, Artificial Sequence
  IgG4 hinge domain
ESKYGPPCPPCP

SEQ ID NO: 4 - protein, 28 aa, Artificial Sequence
  CD28 transmembrane domain
MFWVLVVVGGVLACYSLLVTVAFIIFWV

SEQ ID NO: 5 - protein, 42 aa, Artificial Sequence
  4-1BB costimulatory domain
KRGRKKLLYIFKQPFMRPVQTTQEEDGCSCRFPEEEEGGCEL

SEQ ID NO: 6 - protein, 112 aa, Artificial Sequence
  CD3z signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 24 aa, Artificial Sequence
  T2A ribosomal skipping sequence
LEGGGEGRGSLLTCGDVEENPGPR

SEQ ID NO: 8 - protein, 22 aa, Artificial Sequence
  GMCSF signal peptide
MLLLVTSLLLCELPHPAFLLIP

SEQ ID NO: 9 - protein, 335 aa, Artificial Sequence
  EGFRt
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 10 - protein, 22 aa, Artificial Sequence
  GMCSF signal peptide
MLLLVTSLLLCELPHPAFLLIP

SEQ ID NO: 11 - protein, 243 aa, Artificial Sequence
  CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTV

SEQ ID NO: 12 - protein, 12 aa, Artificial Sequence
  IgG4 hinge domain
ESKYGPPCPPCP

SEQ ID NO: 13 - protein, 28 aa, Artificial Sequence
  CD28 transmembrane domain
MFWVLVVVGGVLACYSLLVTVAFIIFWV

SEQ ID NO: 14 - protein, 41 aa, Artificial Sequence
  CD28 costimulatory domain
RSKRSRGGHSDYMNMTPRRPGPTRKHYQPYAPPRDFAAYRS

SEQ ID NO: 15 - protein, 112 aa, Artificial Sequence; identical to SEQ ID NO: 6
  CD3z signaling domain

SEQ ID NO: 16 - protein, 24 aa, Artificial Sequence
  T2A ribosomal skipping sequence
LEGGGEGRGSLLTCGDVEENPGPR

SEQ ID NO: 17 - protein, 22 aa, Artificial Sequence
  GMCSF signal peptide
MLLLVTSLLLCELPHPAFLLIP

SEQ ID NO: 18 - protein, 335 aa, Artificial Sequence; identical to SEQ ID NO: 9
  EGFRt

SEQ ID NO: 19 - protein, 22 aa, Artificial Sequence
  GMCSF signal peptide
MLLLVTSLLLCELPHPAFLLIP

SEQ ID NO: 20 - protein, 121 aa, Artificial Sequence
  hR12 heavy chain variable domain (VH)
QVQLVESGGALVQPGGSLTLSCKASGFDFSAYYMSWVRQAPGKGLEWIATIYPSSGKTYYAASVQGRFTISADNAKNTVYLQMNSLTAADTATYFCARDSYADDGALFNIWGQGTLVTVSS

SEQ ID NO: 21 - protein, 15 aa, Artificial Sequence
  4(GS)x3 linker
GGGGSGGGGSGGGGS

SEQ ID NO: 22 - protein, 111 aa, Artificial Sequence
  hR12 light chain variable domain (VL)
QLVLTQSPSVSAALGSSAKITCTLSSAHKTDTIDWYQQLAGQAPRYLMYVQSDGSYEKRSGVPDRFSGSSSGADRYLIISSVQADDEADYYCGADYIGGYVFGGGTQLTVG

SEQ ID NO: 23 - protein, 12 aa, Artificial Sequence
  IgG4 hinge domain
ESKYGPPCPPCP

SEQ ID NO: 24 - protein, 28 aa, Artificial Sequence
  CD28 transmembrane domain
MFWVLVVVGGVLACYSLLVTVAFIIFWV

SEQ ID NO: 25 - protein, 42 aa, Artificial Sequence; identical to SEQ ID NO: 5
  4-1BB costimulatory domain

SEQ ID NO: 26 - protein, 112 aa, Artificial Sequence; identical to SEQ ID NO: 6
  CD3z signaling domain

SEQ ID NO: 27 - protein, 24 aa, Artificial Sequence
  T2A ribosomal skipping sequence
LEGGGEGRGSLLTCGDVEENPGPR

SEQ ID NO: 28 - protein, 22 aa, Artificial Sequence
  GMCSF signal peptide
MLLLVTSLLLCELPHPAFLLIP

SEQ ID NO: 29 - protein, 335 aa, Artificial Sequence; identical to SEQ ID NO: 9
  EGFRt

SEQ ID NO: 30 - protein, 22 aa, Artificial Sequence
  GMCSF signal peptide
MLLLVTSLLLCELPHPAFLLIP

SEQ ID NO: 31 - protein, 119 aa, Artificial Sequence
  huLuc63 heavy chain variable domain (VH)
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 32 - protein, 15 aa, Artificial Sequence
  4(GS)x3 linker
GGGGSGGGGSGGGGS

SEQ ID NO: 33 - protein, 107 aa, Artificial Sequence
  huLuc63 light chain variable domain (VL)
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 34 - protein, 228 aa, Artificial Sequence
  IgG4 hinge domain
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 28 aa, Artificial Sequence
  CD28 transmembrane domain
MFWVLVVVGGVLACYSLLVTVAFIIFWV

SEQ ID NO: 36 - protein, 42 aa, Artificial Sequence; identical to SEQ ID NO: 5
  4-1BB costimulatory domain

SEQ ID NO: 37 - protein, 112 aa, Artificial Sequence; identical to SEQ ID NO: 6
  CD3z signaling domain

SEQ ID NO: 38 - protein, 24 aa, Artificial Sequence
  T2A ribosomal skipping sequence
LEGGGEGRGSLLTCGDVEENPGPR

SEQ ID NO: 39 - protein, 22 aa, Artificial Sequence
  GMCSF signal peptide
MLLLVTSLLLCELPHPAFLLIP

SEQ ID NO: 40 - protein, 335 aa, Artificial Sequence; identical to SEQ ID NO: 9
  EGFRt

SEQ ID NO: 41 - protein, 22 aa, Artificial Sequence
  GMCSF signal peptide
MLLLVTSLLLCELPHPAFLLIP

SEQ ID NO: 42 - protein, 119 aa, Artificial Sequence; identical to SEQ ID NO: 31
  huLuc63 heavy chain variable domain (VH)

SEQ ID NO: 43 - protein, 15 aa, Artificial Sequence
  4(GS)x3 linker
GGGGSGGGGSGGGGS

SEQ ID NO: 44 - protein, 107 aa, Artificial Sequence; identical to SEQ ID NO: 33
  huLuc63 light chain variable domain (VL)

SEQ ID NO: 45 - protein, 228 aa, Artificial Sequence; identical to SEQ ID NO: 34
  IgG4 hinge domain

SEQ ID NO: 46 - protein, 28 aa, Artificial Sequence
  CD28 transmembrane domain
MFWVLVVVGGVLACYSLLVTVAFIIFWV

SEQ ID NO: 47 - protein, 41 aa, Artificial Sequence; identical to SEQ ID NO: 14
  CD28 costimulatory domain

SEQ ID NO: 48 - protein, 112 aa, Artificial Sequence; identical to SEQ ID NO: 6
  CD3z signaling domain

SEQ ID NO: 49 - protein, 24 aa, Artificial Sequence
  T2A ribosomal skipping sequence
LEGGGEGRGSLLTCGDVEENPGPR

SEQ ID NO: 50 - protein, 22 aa, Artificial Sequence
  GMCSF signal peptide
MLLLVTSLLLCELPHPAFLLIP

SEQ ID NO: 51 - protein, 335 aa, Artificial Sequence; identical to SEQ ID NO: 9
  EGFRt

(19) United States
(12) Patent Application Publication
(10) Pub. No.: US 2021/0169880 A1
(43) Pub. Date: Jun. 10, 2021

(54) CONTROL AND MODULATION OF THE FUNCTION OF GENE-MODIFIED CHIMERIC ANTIGEN RECEPTOR T CELLS WITH DASATINIB AND OTHER TYROSINE KINASE INHIBITORS

(71) Applicant: JULIUS-MAXIMILIANS-UNIVERSITÄT WÜRZBURG, Würzburg (DE)

(72) Inventors: Michael HUDECEK, Höchberg (DE); Katrin MESTERMANN, Rimpar (DE)

(21) Appl. No.: 16/767,545

(22) PCT Filed: Dec. 7, 2018
(86) PCT No.: PCT/EP2018/084018

§ 371 Date: May 27, 2020

(30) Foreign Application Priority Data
Dec. 7, 2017 (EP) 17205922.2

(51) Int. Cl. A61K 31/506; A61P 35/00; C07K 14/705; A61K 35/17

(52) U.S. Cl. CPC A61K 31/506; A61K 35/17; C07K 14/705; A61P 35/00

(57) ABSTRACT

The invention relates to the immunomodulatory features of dasatinib and other tyrosine kinase inhibitors towards genetically modified immune cells. The invention encompasses the indication of dasatinib and other tyrosine kinase inhibitors as an immune cell inhibitor as well as an enhancer of immune cells depending on the dosing and schedule of treatment, the administration routes, the susceptible receptor variants and the treatable cell types which can be used for immunotherapy.

### FIELD OF THE INVENTION

The invention relates to the use of dasatinib and other tyrosine kinase inhibitors to control and modulate the function of gene-modified chimeric antigen receptor (CAR)-T cells in cancer immunotherapy. The invention comprises the use of dasatinib and other tyrosine kinase inhibitors to enhance safety through the prevention and treatment of potentially life-threatening side effects that may occur during CAR-T cell immunotherapy, and the use of dasatinib to augment the anticancer potency and efficacy of CAR-T cell immunotherapy.

### BACKGROUND OF THE INVENTION

Adoptive immunotherapy with T cells that were engineered by transient or stable gene transfer to express a chimeric antigen receptor (CAR) is under pre-clinical and clinical investigation as a highly innovative and highly effective novel treatment for advanced chemotherapy- and radiotherapy-refractory malignancies in hematology and oncology.

CARs are synthetic designer receptors, commonly comprised of an extracellular antigen-binding moiety that binds to a surface molecule or structure on tumor cells; a spacer and transmembrane domains that anchors the receptor on the T cell surface; and an intracellular signaling module, most commonly a CD3 zeta domain in cis with a costimulatory moiety derived from CD28 or 4-1BB, to activate and stimulate the CAR-T cell after binding of the respective target molecule or structure. In addition, alternative CAR designs comprising NKG2D domains, the T-cell receptor constant domains, and other CD3 subunits are being developed. At present, the process of antigen binding, signal generation and transduction, subsequent T cell activation and stimulation of CARs is incompletely understood, owing at least in part to the fact that CARs comprise domains (e.g. signaling domains like CD3 zeta, CD28 and 4-1BB) that occur in endogenous T cells, but are assembled in the CAR construct in a new and artificial way.

Clinical proof-of-concept for the efficacy of CAR-T cell immunotherapy has been accomplished with CAR-T cells specific for the CD19 molecule (CD19 CAR-T cells) that is expressed on malignant cells in B-cell leukemia and lymphoma [1]-[3] and recently also with CAR-T cells specific for the B cell maturation antigen (BCMA) (BCMA CAR-T cells) that is expressed in multiple myeloma (MM) [4]. Adoptive transfer of autologous or allogeneic CD19 CAR-T cells has induced durable complete and partial responses in patients with acute lymphoblastic leukemia (ALL), chronic lymphocytic leukemia (CLL), non-Hodgkin lymphoma (NHL), and MM. CD19 CAR-T cells have been approved by the FDA for the treatment of relapsed/refractory ALL and NHL in 2017. Adoptive transfer of BCMA CAR-T cells has induced durable complete and partial responses in patients with MM. At present, numerous clinical trials with CAR-T cells targeting CD19, BCMA and other antigens are ongoing at cancer centers world-wide.

Even though CAR-T cell therapy is being appraised as a remarkably potent and highly effective novel anticancer treatment, there are significant concerns related to safety. The clinical use of CAR-T cells (including CD19 CAR-T cells and BCMA CAR-T cells) has disclosed a number of acute and chronic, potentially life-threatening and in some cases, fatal, side effects that have thus far limited the clinical use of CAR-T cells, and restricted their application to medically fit patients at highly specialized cancer centers with in-depth experience in bone marrow transplantation and immunotherapy. These side effects may be due to (but not limited to): i) the strong activation and subsequent cytokine release from CAR-T cells after adoptive transfer into the patient due to the presence of a large number of tumor cells that express the respective target antigen (cytokine release syndrome CRS); ii) the activation of other immune cells in the patient's body that take up the tumor cell debris that accumulates as a result of tumor cell killing by CAR-T cells (e.g. macrophage activation syndrome, MAS); iii) on-target recognition and elimination of normal cells in the patient's body that express the respective target antigen (e.g.: depletion of normal B cells by CD19 CAR-T cells); iv) off-target recognition of normal (or malignant) cells in the patient's body that do not express the respective target antigen of the CAR; v) the rejection of CAR-T cells due to an immune response of the patient's immune system against the transferred CAR-T cells, either due to recognition of the CAR construct or the T cell if the T cell is derived from an allogeneic donor; vi) inadvertent activation of CAR-T cells if the CAR construct harbors motifs that are recognized by endogenous immune cells (e.g. Fc-motif in Ig-derived CAR spacer domain); vii) tonic signaling and activation of CAR-T cells independent from stimulation with antigen.

A severe side effect of CAR-T cell therapy is CRS. CRS symptoms are caused by elevated levels of pro-inflammatory cytokines including GM-CSF, IFN-γ, TNF-α, IL-2, IL-6, IL-8, IL-10 [5] and commonly start with development of fever, often within hours to few days after CAR-T cell transfer. CRS symptoms may include tachycardia/hypotension, malaise, fatigue, myalgia, nausea, anorexia and capillary leak and may result in multi-organ failure [6]. The risk of developing CRS correlates with the total dose of CAR-T cells that are administered, and the tumor burden prior to CAR-T cell therapy [5], [7]. CRS is major cause of morbidity and mortality in CAR-T cell therapy.

At present, the ability to prevent and treat clinical CRS, and to prevent or treat other side effects in the context of CAR-T cell therapy is very limited. At present, there is no means to effectively control the function of CAR-T cells after infusion into the patient. CAR-T cells are a ‘living drug’, i.e. after infusion into the patient they become part of the patient's immune system, expand and subsequently contract in the patient, and may persist long-term as memory CAR-T cells that prevent tumor relapse. It has been demonstrated that CAR-T cell engraftment and persistence (area under the curve, AUC) correlates with therapeutic efficacy. In this regard, CAR-T cells are different form conventional drugs that are either eliminated, metabolized or decay in the patient with a predictable and consistent half-life.

At present, there are three major strategies to mitigate CRS, and to treat or prevent side effects of CAR-T cell therapy. 1) Tocilizumab: It has been shown that Interleukin-6 (IL-6) plays a critical role in CRS and therefore, blockade of the IL-6 receptor (IL-6R) through the anti-IL-6R antibody tocilizumab is often attempted, and has been shown to mitigate CRS in a significant proportion of patients. However, this intervention does not exert a direct effect on CAR-T cells and is rather a symptomatic treatment. 2) Steroids: It is commonly attempted to mitigate CRS or other CAR-T cell-mediated side effects through administration of Dexamethasone or Prednisone. However, their ability to control CRS or other side effects is low. Because steroids are known to be immunosuppressive, their use in the context of CAR-T cell therapy has raised concerns that they may negatively influence the therapeutic effect of CAR-T cells. 3) Suicide genes and depletion markers: Some CAR-T cell products are equipped with ‘emergency breaks’, i.e. suicide genes like inducible Caspase 9 (iCasp9) that can be triggered by a dimerizer drug to induce apoptosis of CAR-T cells. A limitation is that this strategy works well for CAR-T cells that express high levels of this suicide gene, but is ineffective in low expressers [8] or depletion markers like EGFRt or CD20t that can be triggered by antibodies that induce antibody-dependent cellular cytotoxicity (ADCC) or complement-dependent cytotoxicity (CDC) to remove CAR-T cells [9]. However, these antibody-dependent depletion markers may only work if the patient's immune system is unaltered which is often not the case after intensive prior chemotherapy, or due to depletion of normal immune cells as part of on-target recognition of the CAR. A major concern with suicide genes and depletion markers is that they eliminate CAR-T cells and terminate their therapeutic effect. This is of particular concern, because due to the immunogenicity of current CAR constructs, second infusions are often not possible (because patients develop an immune response and reject CAR-T cells at the time of second infusion). As a consequence, there is at present no reported case where iCasp9 or EGFRt have been triggered in the context of CAR-T cell immunotherapy.

It has recently been shown that patients that received CAR-T cells and are at high risk of developing CRS and/or neurotoxicity can be identified by measuring serum cytokines including but not limited to IFN-γ, IL-6, MCP1, and measuring viral signs like body temperature [2], [10]. If one could control (and intermittently pause) the function of CAR-T cells in such patients, it would be possible to mitigate or prevent these toxicities.

At present, there is an unmet need for a method to control the function of CAR-T cells after administration to the patient, to prevent or treat CRS or other side effects, while preserving the subsequent anticancer effect of the CAR-T cell product.

Another challenge in CAR-T cell cancer immunotherapy is that in a subset of patients, this treatment is ineffective and does not lead to the desired therapeutic response. There are several mechanisms that may lead to inefficacy of CAR-T cell therapy (including, but not limited to): 1) CAR-T cells are exhausted because of constant exposure to antigen and ensuing constant signaling from the CAR, especially in patients with high tumor burden, and patients with solid tumors. 2) CAR-T cells are exhausted after their manufacture ex vivo and subsequently fail to engraft, expand, persist, proliferate and function against cancer cells in the patient's body; 3) CAR-T cells are exhausted and undergo activation-induced cell death (AICD) due to tonic signaling from the CAR construct; 4) CAR-T cells express check-point molecules, including but not limited to PD-1, that inhibit their viability, proliferation and function against cancer cells. The programmed cell death protein 1 (PD-1) is expressed on the surface of T cells. PD-1 promotes apoptosis in T cells upon binding to its ligand, PD-L1 which is commonly expressed on cancer cells and in the tumor microenvironment. Blockade of the PD-1_PD-L1 axis through check-point blockers, i.e, anti.PD-1 or anti-PD-L1 antibodies is being pursued as a strategy to augment the function of endogenous and CAR-modified T cells in cancer immunotherapy [11].

At present, there is an unmet need for means to improve the viability and function of CAR-T cells in patients that do not respond to CAR-T cell immunotherapy.

The tyrosine-kinase inhibitor dasatinib (©Sprycel) has been developed as an inhibitor of the BCR-ABL fusion protein [12] which is commonly expressed in Philadelphia-chromosome positive (Ph+) chronic myeloid leukemia (CML) [13] and in about 20% of cases in ALL [14]. Since 2010, dasatinib is approved for the first-line treatment of Ph+ ALL and CML. In addition, dasatinib has been shown to block the ATP binding sites of the SRC kinase Lck, which is involved in the signaling cascade of conventional T cells after stimulation through the endogenous, physiologic T-cell receptor[15]-[18].

### DESCRIPTION OF THE INVENTION

The present invention utilizes to the inventors' finding of the previously unknown and unexpected ability of the tyrosine kinase inhibitor dasatinib to block the function of CAR-T cells through continuous administration of the drug. Further, the invention utilizes the inventors' finding of the previously unknown and unexpected ability of the tyrosine kinase inhibitor dasatinib to augment the function of CAR-T cells through intermittent administration of the drug.

According to the invention, the continuous administration of dasatinib confers a rapid and complete blockade of CAR-T cell function. This blockade remains effective as long as CAR-T cells are continuously exposed to dasatininb at a concentration above a certain threshold. This blockade is effective in non-activated and already activated CAR-T cells. This blockade is effective in both CD8+ killer and CD4+ helper (and regulatory) T cells. Further, this blockade is effective independent from the antigen specificity of the CAR, and independent from the particular design of the CAR with respect to the antigen.binding domain, the extracellular spacer domain, and the intracellular signaling and costimulatory moiety. Further, this blockade is effective as long as exposure to dasatinib is maintained, but rapidly and completely reversible once exposure to dasatinib is discontinued. Further, this blockade does not affect the viability of CAR-T cells, and does not affect the ability of CAR-T cells to exert their anticancer function once exposure to dasatinib has been discontinued. According to the invention, the ability of dasatinib to control the function of CAR-T cells is distinct from and superior to the ability of steroids to control and inhibit the function of CAR-T cells. According to the invention, the ability of dasatinib to block CAR-T cell function can be exploited to enhance the safety of CAR-T cell therapy, including but not limited to preventing and treating CRS.

According to the invention, the intermittent administration of dasatinib can be exploited to augment the antitumor function of CAR-T cells. This augmentation is due to an increase in CAR-T cell viability and function upon intermittent exposure to dasatinib. Further, this augmentation is due to an increase in engraftment, proliferation and persistence of CAR-T cells upon intermittent exposure to dasatinib. Further, this augmentation is due to superior signaling of the CAR upon intermittent exposure to dasatinib. Further, this augmentation is due to a decrease in expression of inhibitory immune check-point molecules on CAR-T cells, including but not limited to PD-1, upon intermittent exposure to dasatinib. Intermittent administration shall comprise any use of dasatinib at intervals of constant or variable length where the concentration of dasatininb is not contiguously above the concentration required to block CAR-T cell function.

The present invention is exemplified by the following preferred embodiments:
1. A composition for use in a method for the treatment of cancer in a patient, the composition comprising a tyrosine kinase inhibitor;
   wherein in the method, the composition is to be administered to the patient, and wherein the method is a method for treating cancer comprising immunotherapy.
2. The composition of item 1 for use of item 1, wherein the immunotherapy is adoptive immunotherapy.
3. The composition of any one of items 1 to 2 for use of any one of items 1 to 2, wherein said immunotherapy is immunotherapy with immune cells.
4. The composition of item 3 for use of item 3, wherein said immunotherapy is immunotherapy with immune cells expressing a chimeric antigen receptor.
5. The composition of item 4 for use of item 4, wherein said chimeric antigen receptor is capable of binding to an antigen.
6. The composition of item 5 for use of item 5, wherein said chimeric antigen receptor is capable of binding to a cell surface antigen.
7. The composition of any one of items 5 to 6 for use of any one of items 5 to 6, wherein said antigen is a cancer antigen.
8. The composition of any one of items 5 to 7 for use of any one of items 5 to 7, wherein said antigen is selected from the group consisting of CD4, CD5, CD10, CD19, CD20, CD22, CD27, CD30, CD33, CD38, CD44v6, CD52, CD64, CD70, CD72, CD123, CD135, CD138, CD220, CD269, CD319, ROR1, ROR2, SLAMF7, BCMA, αvβ3-Integrin, α4β1-integrin, LILRB4, EpCAM-1, MUC-1, MUC-16, L1-CAM, c-kit, NKG2D, NKG2D-Ligand, PD-L1, PD-L2, Lewis-Y, CAIX, CEA, c-MET, EGFR, EGFRvIII, ErbB2, Her2, FAP, FR-a, EphA2, GD2, GD3, GPC3, IL-13Ra, Mesothelin, PSMA, PSCA, VEGFR, and FLT3.
9. The composition of item 8 for use of item 8, wherein said antigen is selected from the group consisting of CD19, CD20, CD22, CD123, SLAMF7, ROR1, BCMA, and FLT3.
10. The composition of item 9 for use of item 9, wherein said antigen is CD19
11. The composition of item 9 for use of item 9, wherein said antigen is ROR1.
12. The composition of item 9 for use of item 9, wherein said antigen is BCMA.
13. The composition of item 9 for use of item 9, wherein said antigen is FLT3.
14. The composition of item 9 for use of item 9, wherein said antigen is CD20.
15. The composition of item 9 for use of item 9, wherein said antigen is CD22.
16. The composition of item 9 for use of item 9, wherein said antigen is CD123.
17. The composition of item 9 for use of item 9, wherein said antigen is SLAMF7.
18. The composition of any one of items 5 to 17 for use of any one of items 5 to 17, wherein said cancer comprises cancer cells which express said antigen.
19. The composition of any one of items 4 to 18 for use of any one of items 4 to 18, wherein said chimeric antigen receptor comprises a costimulatory domain selected from the group consisting of the CD27, CD28, 4-1BB, ICOS, DAP10, NKG2D, MyD88 and OX40 costimulatory domains.
20. The composition of item 19 for use of item 19, wherein said chimeric antigen receptor comprises a CD28 costimulatory domain.
21. The composition of item 19 for use of item 19, wherein said chimeric antigen receptor comprises a 4-1BB costimulatory domain.
22. The composition of item 19 for use of item 19, wherein said chimeric antigen receptor comprises an OX40 costimulatory domain.
23. The composition of any one of items 3 to 22 for use of any one of items 3 to 22, wherein said immune cells are lymphocytes.
24. The composition of any one of items 3 to 23 for use of any one of items 3 to 23, wherein said immune cells are B lymphocytes or T lymphocytes.
25. The composition of item 24 for use of item 24, wherein said immune cells are T lymphocytes.
26. The composition of item 25 for use of item 25, wherein said immune cells are CD4+ and/or CD8+ T lymphocytes.
27. The composition of item 26 for use of item 26, wherein said immune cells are CD4+ T lymphocytes.
28. The composition of item 26 for use of item 26, wherein said immune cells are CD8+ T lymphocytes.
29. The composition of any one of items 3 to 28 for use of any one of items 3 to 28, wherein said immune cells are selected from the group consisting of CD8+ killer T cells, CD4+ helper T cells, naïve T cells, memory T cells, central memory T cells, effector memory T cells, memory stem T cells, invariant T cells, NKT cells, cytokine induced killer T cells, gamma/delta T cells, natural killer cells, monocytes, macrophages, dendritic cells, and granulocytes.
30. The composition of any one of items 1 to 29 for use of any of items 1 to 29, wherein said tyrosine kinase inhibitor is a Src kinase inhibitor.
31. The composition of any one of items 1 to 30 for use of any one of items 1 to 30, wherein said tyrosine kinase inhibitor is an inhibitor of kinases upstream of NFAT.
32. The composition of any one of items 1 to 31 for use of any one of items 1 to 31, wherein said tyrosine kinase inhibitor is an Lck kinase inhibitor.
33. The composition of any one of items 1 to 32 for use of any one of items 1 to 32, wherein said tyrosine kinase inhibitor is selected from the group consisting of dasatinib, saracatinib, bosutinib, nilotinib, and PP1-inhibitor.
34. The composition of item 33 for use of item 33, wherein said tyrosine kinase inhibitor is dasatinib.
35. The composition of item 33 for use of item 33, wherein said tyrosine kinase inhibitor is bosutinib.
36. The composition of item 33 for use of item 33, wherein said tyrosine kinase inhibitor is PP1-inhibitor.
37. The composition of item 33 for use of item 33, wherein said tyrosine kinase inhibitor is nilotinib.
38. The composition of any one of items 3 to 37 for use of any one of item 3 to 37, wherein said tyrosine kinase inhibitor causes inhibition of said immune cells.
39. The composition of item 38 for use of item 38, wherein said inhibition is an inhibition of cell mediated effector functions of said immune cells.
40. The composition of any one of items 38 to 39 for any one of use of items 38 to 39, wherein said inhibition of said immune cells is an inhibition of their
   I) cytolytic activity; and/or
   II) cytokine secretion; and/or
   III) proliferation.
41. The composition of any one of items 38 to 40 for use of any one of items 38 to 40, wherein said inhibition comprises inhibition of PD1 expression in said immune cells.
42. The composition of any one of items 38 to 41 for use of any one of items 38 to 41, wherein said inhibition comprises inhibition of cytokine secretion of said immune cells of one or more cytokines selected from the group consisting of GM-CSF, IFN-γ, IL-2, IL-4, IL-5, IL-6, IL-8, and IL-10.
43. The composition of any one of items 38 to 42 for use of any one of items 38 to 42, wherein said inhibition comprises inhibition of IFN-γ and/or IL-2 secretion of said immune cells.
44. The composition of item 43 for use of item 43, wherein said inhibition comprises inhibition of IFN-γ secretion of said immune cells.
45. The composition of item 43 for use of item 43, wherein said inhibition comprises inhibition of IL-2 secretion of said immune cells.
46. The composition of items 38 to 45 for use of items 38 to 45, wherein said inhibition is a partial inhibition or a complete inhibition.
47. The composition of any one of items 38 to 46 for use of any one of items 38 to 46, wherein said inhibition does not decrease the viability of said immune cells.
48. The composition of item 47 for use of item 47, wherein said inhibition does not decrease the viability of said immune cells for a given time period during which said composition is administered to said patient, wherein said time period is 1 hour, preferably 2 hours, preferably 3 hours, preferably 4 hours, preferably 5 hours, preferably 6 hours, preferably 8 hours, preferably 12 hours, preferably 18 hours, preferably 1 day, preferably 2 days, more preferably 3 days, even more preferably 7 days, even more preferably 2 weeks, even more preferably 3 weeks, even more preferably 4 weeks, even more preferably 2 months, even more preferably 3 months, even more preferably 6 months.
49. The composition of any one of items 38 to 48 for use of any one of items 38 to 48, wherein said inhibition is reversible.
50. The composition of item 49 for use of item 49, wherein said inhibition is reversed after said composition has not been administered to said patient for a given amount of time.
51. The composition of item 50 for use of item 50, wherein said given amount of time is 3 days, preferably 2 days, more preferably 24 hours, even more preferably 18 hours, even more preferably 12 hours, even more preferably 8 hours, even more preferably 6 hours, even more preferably 4 hours, even more preferably 3 hours, even more preferably 2 hours, even more preferably 90 minutes, even more preferably 60 minutes, even more preferably 30 minutes.
52. The composition of any one of items 1 to 51 for use of any one of items 1 to 51, wherein said composition is to be administered continuously or intermittently.
53. The composition of item 52 for use of item 52, wherein said composition is to be administered continuously.
54. The composition of item 52 for use of item 52, wherein said composition is to be administered intermittently.
55. The composition of any one of items 1 to 54 for use of any one of items 1 to 54, wherein the composition is to be administered such that after initial administration of said composition the serum levels of said tyrosine kinase inhibitor are maintained at or above a threshold serum level during the duration of said treatment.
56. The composition of any one of items 1 to 55 for use of any one of items 1 to 55, wherein in the method, the composition is to be administered such that after initial administration of said composition the serum levels of said tyrosine kinase inhibitor are maintained at least once above a threshold serum level and a least once below the same threshold serum level during the duration of said treatment.
57. The composition of any one of items 55 to 56 for use of any one of items 55 to 56, wherein said threshold serum level is within the range of 0.1 nM-1 μM, preferably 1 nM-500 nM, more preferably 5 nM-100 nM, even more preferably 10 nM-75 nM, even more preferably 25 nM-50 nM.
58. The composition of item 57 for use of item 57, wherein said threshold serum level is 50 nM.
59. The composition of any one of items 55 to 58 for use of any one of items 55 to 58, wherein said threshold serum level is the minimum serum level at which said inhibition of said immune cells is a complete inhibition of their
   I) cytolytic activity; and/or
   II) cytokine secretion; and/or
   III) proliferation.
60. The composition of any one of items 1 to 59 for use of any one of items 1 to 59, wherein said treatment of cancer has an improved clinical outcome compared to said immunotherapy against said cancer alone.
61. The composition of any one of items 1 to 60 for use of any one of items 1 to 60, wherein said use is a use for mitigating or preventing toxicity associated with said immunotherapy against said cancer.
62. The composition of any one of items 1 to 61 for use of any one of items 1 to 61, wherein said use is a use for decreasing tumor burden in said patient compared to said immunotherapy against said cancer alone.
63. The composition of any one of items 1 to 62 for use of any one of items 1 to 62, wherein said use in the treatment of cancer does not decrease the therapeutic efficacy of said immunotherapy against said cancer compared to said immunotherapy against said cancer alone.
64. The composition of any one of items 1 to 63 for use of any one of items 1 to 63, wherein said use in the treatment of cancer is a use for increasing the therapeutic efficacy of said immunotherapy against said cancer compared to said immunotherapy against said cancer alone.
65. The composition of any one of items 1 to 64 for use of any one of items 1 to 64, wherein said use in the treatment of cancer is a use for decreasing the morbidity and mortality of said immunotherapy against said cancer compared to said immunotherapy against said cancer alone.
66. The composition of any one of items 1 to 65 for use of any one of items 1 to 65, wherein said use in the treatment of cancer is a use for increasing the anti-tumor efficacy of said immunotherapy against said cancer compared to said immunotherapy against said cancer alone.
67. The composition of any one of items 3 to 66 for use of any one of items 3 to 66, wherein said use in the treatment of cancer is a use for increasing the engraftment and/or persistence of said immune cells in said immunotherapy against said cancer compared to the engraftment and/or persistence of said immune cells in said immunotherapy against said cancer alone.
68. The composition of any one of items 3 to 67 for use of any one of items 3 to 67, wherein said use in the treatment of cancer is a use for increasing the engraftment of said immune cells in said immunotherapy compared to the engraftment of said immune cells in a method comprising said immunotherapy against said cancer alone.
69. The composition of any one of items 3 to 68 for use of any one of items 3 to 68, wherein said use is a use for decreasing the exhaustion of said immune cells in said immunotherapy against said cancer compared to the exhaustion of said immune cells in a method comprising said immunotherapy against said cancer alone.
70. The composition of any one of items 1 to 69 for use of any one of items 1 to 69, wherein said composition is to be administered
   I) before said treatment of cancer by immunotherapy; and/or
   II) concurrently to said treatment of cancer by immunotherapy; and/or
   III) after said treatment of cancer by immunotherapy.
71. The composition of item 70 for use of item 70, wherein said composition is to be administered before said treatment of cancer by immunotherapy.
72. The composition of item 70 for use of item 70, wherein said composition is to be administered concurrently to said treatment of cancer by immunotherapy.
73. The composition of item 70 for use of item 70, wherein said composition is to be administered after said treatment of cancer by immunotherapy.
74. The composition of item 70 for use of item 70, wherein said composition is to be administered before said treatment of cancer by immunotherapy and concurrently to said treatment of cancer by immunotherapy.
75. The composition of item 70 for use of item 70, wherein said composition is to be administered before said treatment of cancer by immunotherapy and after said treatment of cancer by immunotherapy.
76. The composition of item 70 for use of item 70, wherein said composition is to be administered concurrently to said treatment of cancer by immunotherapy and after said treatment of cancer by immunotherapy.
77. The composition of item 70 for use of item 70, wherein said composition is to be administered before said treatment of cancer by immunotherapy, concurrently to said treatment of cancer by immunotherapy, and after said treatment of cancer by immunotherapy.
78. The composition of any one of items 3 to 77 for use of any one of items 3 to 77, wherein said use is a use for preventing activation of said immune cells in said immunotherapy.
79. The composition of item 78 for use of item 78, wherein said immune cells are resting immune cells.
80. The composition of any one of items 3 to 79 for of any one of items 3 to 79, wherein said immune cells are of human origin.
81. The composition of item 80 for use of item 80, wherein said immune cells of human origin are primary human cells.
82. The composition of item 81 for use of item 81, wherein said primary human cells are primary human T lymphocytes.
83. The composition of any one of items 80 to 82 for use of any one of items 80 to 82, wherein said immune cells of human origin are allogeneic cells with respect to said patient.
84. The composition of any one of items 80 to 82 for use of any one of item 80 to 82, wherein said immune cells of human origin are syngeneic cells with respect to said patient.
85. The composition of any one of items 4 to 84 for use of any one of items 4 to 84, wherein said immune cells are immune cells which transiently or stably express said chimeric antigen receptor.
86. The composition of any one of items 4 to 85 for use of any one of items 4 to 85, wherein said chimeric antigen receptor is of first, second, or third generation.
87. The composition of any one of items 5 to 86 for use of any one of items 5 to 86, wherein said chimeric antigen receptor comprises a single chain variable fragment, preferably wherein said single chain variable fragment is capable of binding to said antigen.
88. The composition of any one of items 5 to 86 for use of any one of items 5 to 86, wherein said chimeric antigen receptor comprises a ligand or fragment thereof, wherein said ligand or fragment thereof is capable of binding to said antigen.
89. The composition of any one of items 5 to 88 for use of any one of items 5 to 88, wherein said chimeric antigen receptor comprises a signaling domain comprising one or more domains selected from the group consisting of CD3 zeta, CD3 epsilon, CD3 gamma, T-cell receptor alpha chain, T-cell receptor beta chain, T-cell receptor delta chain, and T-cell receptor gamma chain.
90. The composition of any one of items 1 to 89 for use of any one of items 1 to 89, wherein said cancer is a cancer associated with a higher risk of morbidity and mortality in said immunotherapy.
91. The composition of any one of items 1 to 90 for use of any one of items 1 to 90, wherein said cancer comprises cells which express one or more checkpoint molecules, which are preferably selected from the group consisting A2AR, B7-H3, B7-H4, BTLA, CTLA-4, IDO, KIR, LAG3, PD-L1, PD-L2, TIM-3, and VISTA.
92. The composition of item 91 for use of item 91, wherein said cancer comprises cells which express PD-1.
93. The composition of any one of items 1 to 92 for use of any one of items 1 to 92, wherein said cancer is a cancer selected from the group consisting of carcinoma, sarcoma, myeloma, leukemia, and lymphoma.
94. The composition of item 93 for use of item 93, wherein said cancer is myeloma.
95. The composition of item 93 for use of item 93, wherein said cancer is leukemia.
96. The composition of item 93 for use of item 93, wherein said cancer is lymphoma.
97. The composition of item 93 for use of item 93, wherein said cancer is carcinoma, preferably wherein said cancer is a carcinoma selected from the group consisting of breast cancer, lung cancer, colorectal cancer, and pancreatic cancer.
98. The composition of item 95 for use of item 95, wherein said leukemia is B-cell leukemia, T-cell leukemia, myeloid leukemia, acute lymphoblastic leukemia, or chronic myeloid leukemia.
99. The composition of item 96 for use of item 96, wherein said lymphoma is non-Hodgkin lymphoma, Hodgkin lymphoma, or B-cell lymphoma.
100. The composition of any one of items 1 to 99 for use of any one of items 1 to 99, wherein said cancer is a cancer characterized as
   I) CD19 positive; and/or
   II) BCMA positive; and/or
   III) ROR1 positive; and/or
   IV) FLT3 positive; and/or
   V) CD20 positive; and/or
   VI) CD22 positive; and/or
   VII) CD123 positive; and/or
   VIII) SLAMF7 positive.
101. The composition of item 100 for use of item 100, wherein said cancer is CD19 positive.
102. The composition of item 100 for use of item 100, wherein said cancer is BCMA positive.
103. The composition of item 100 for use of item 100, wherein said cancer is ROR1 positive.
104. The composition of item 100 for use of item 100, wherein said cancer is FLT3 positive.
105. The composition of item 100 for use of item 100, wherein said cancer is CD20 positive.
106. The composition of item 100 for use of item 100, wherein said cancer is CD22 positive.
107. The composition of item 100 for use of item 100, wherein said cancer is CD123 positive.
108. The composition of item 100 for use of item 100, wherein said cancer is SLAMF7 positive.
109. The composition of any one of items 1 to 108 for use of any one of items 1 to 108, wherein said patient is a patient that is not eligible for said treatment of said cancer by said immunotherapy alone.
110. The composition of any one of items 1 to 109 for use of any one of items 1 to 109, wherein said patient is a patient that is not eligible for conventional adoptive immunotherapy with T cells expressing a chimeric antigen receptor.
111. The composition of any one of items 1 to 110 for use of any one of items 1 to 110, wherein said patient has an increased risk of developing cytokine release syndrome.
112. The composition of any one of items 1 to 111 for use of any one of items 1 to 111, wherein said patient has an increased risk of developing neurotoxic side effects associated with said immunotherapy.
113. The composition of any one of items 1 to 112 for use of any one of items 1 to 112, wherein said patient has an increased risk of developing on-target/off-tumor effects associated with said immunotherapy.
114. The composition of any one of items 1 to 113 for use of any one of items 1 to 113, wherein said patient has elevated serum levels of one or more cytokines selected from the group of IFN-γ, IL-6, and MCP1.
115. The composition of any one of items 1 to 114 for use of any one of items 1 to 114, wherein said patient is a patient that has developed an immune response to said immunotherapy, wherein said immune response is a side effect of said immunotherapy against said cancer.
116. The composition of any one of items 1 to 115 for use of any one of items 1 to 115, wherein said method for treatment is a method for treatment in combination with allogeneic or autologous hematopoietic stem cell transplantation.
117. The composition of any one of items 1 to 116 for use of any one of items 1 to 116, wherein said composition further comprises a pharmaceutically acceptable carrier.
118. The composition of any one of items 1 to 117 for use of any one of items 1 to 117, wherein said composition is to be administered by a route other than oral administration.
119. The composition of any one of items 1 to 118 for use of any one of items 1 to 118, wherein said cancer is a cancer other than chronic myeloid leukemia and acute lymphoblastic leukemia.
120. A composition for use in a method for the treatment of one or more side effects associated with immunotherapy in a patient; wherein the composition comprises a tyrosine kinase inhibitor;
   and wherein in the method, the composition is to be administered to the patient.
121. The composition of item 120 for use of item 120, wherein said immunotherapy is an immunotherapy as defined in any one of items 2 to 17 and 19 to 29.
122. The composition of items 120 to 121 for use of items 120 to 121, wherein said cancer is a cancer as defined in any one of items 18, 90 to 108, and 119.
123. The composition of items 120 to 122 for use of items 120 to 122, wherein said patient is a patient as defined in any one of items 109 to 115.
124. The composition of items 120 to 123 for use of items 120 to 123, wherein said use is a use as defined in any one of items 1 to 119.
125. The composition of any one of items 120 to 124 for the use of any one of items 120 to 124, wherein said one or more side effects associated with immunotherapy are selected from the group consisting of:
   I) cytokine release syndrome, and/or
   II) macrophage activation syndrome, and/or
   III) off-target toxicity, and/or
   IV) on-target/off-tumor recognition of normal and/or malignant cells, and/or
   V) rejection of immunotherapy cells, and/or
   VI) inadvertent activation of immunotherapy cells, and/or
   VII) tonic signaling and activation of immunotherapy cells, and/or
   VIII) neurotoxicity, and/or
   IX) tumor lysis syndrome.
126. The composition of item 125 for use of item 125, wherein said side effect associated with immunotherapy is cytokine release syndrome.
127. The composition of item 125 for use of item 125, wherein said side effect associated with immunotherapy is off-target toxicity.
128. The composition of item 125 for use of item 125, wherein said side effect associated with immunotherapy is on-target/off-tumor recognition of normal and/or malignant cells.
129. The composition of item 125 for use of item 125, wherein said side effect associated with immunotherapy is rejection of immunotherapy cells.
130. The composition of item 125 for use of item 125, wherein said side effect associated with immunotherapy is inadvertent activation of immunotherapy cells.
131. The composition of item 125 for use of item 125, wherein said side effect associated with immunotherapy is tonic signaling and activation of immunotherapy cells.
132. The composition of item 125 for use of item 125, wherein said side effect associated with immunotherapy is neurotoxicity.
133. The composition of item 125 for use of item 125, wherein said side effect associated with immunotherapy is tumor lysis syndrome.
134. The composition of item 126 or use of item 126, wherein said cytokine release syndrome is characterized by elevated cytokine serum levels of one or more cytokines selected from the group consisting of GM-CSF, FN-y, IL-2, IL-4, IL-5, IL-6, IL-8, and IL-10.
135. The composition of item 134 for use of item 134, wherein said use is a use for causing a reduction of one or more of said elevated cytokine serum levels.
136. The composition of any one of items 134 to 135 for use of any one of items 134 to 135, wherein said cytokine release syndrome is caused by said immunotherapy.
137. A composition for use in a method for modulating cells expressing a chimeric antigen receptor in immunotherapy for treating of cancer in a patient; wherein the composition comprises a tyrosine kinase inhibitor;
   and wherein in the method, the composition is to be administered to the patient.
138. The composition of item 137 for use of item 137, wherein said immunotherapy is an immunotherapy as defined in any one of items 2 to 17, 19 to 29, and 125 to 136.
139. The composition of any one of items 137 to 138 for use of any one of items 137 to 138, wherein said cancer is a cancer as defined in any one of items 18, 90 to 108, and 119.
140. The composition of any one of items 137 to 139 for use of any one of items 137 to 139, wherein said patient is a patient as defined in any one of items 109 to 115.
141. The composition of any one of items 137 to 140 for use of any one of items 137 to 140, wherein said use is a use as defined in any one of items 1 to 136.
142. A composition, comprising:
   I) An immune cell, and
   II) A tyrosine kinase inhibitor.
143. The composition of item 142, wherein said immune cell is an immune cell as defined in any one of items 3 to 17, 19 to 29, and 79 to 89.
144. The composition of any one of items 142 to 143, wherein said tyrosine kinase inhibitor is a tyrosine kinase inhibitor as defined in any one of items 30 to 59.
145. The composition of any one of items 142 to 144, wherein the composition comprises a pharmaceutically acceptable carrier.
146. A combination of:
   I) An immune cell, and
   II) A tyrosine kinase inhibitor, for a use as defined in any one of items 1 to 141.
147. The combination of item 144, wherein said immune cell is as immune cell as defined in any one of items 3 to 17, 19 to 29, and 79 to 89.
148. The combination of any one of items 146 to 147, wherein said tyrosine kinase inhibitor is a tyrosine kinase inhibitor as defined in any one of items 30 to 59.

### BRIEF DESCRIPTION OF THE DRAWINGS

FIG. 1: Car Constructs.

scFv: single chain variable fragment (VH-(G₄S)₃ linker-VL). IgG4-FC Hinge: Hinge domain of immunoglobulin G4. CD28: CD28 costimulatory domain. 4-1BB: 4-1BB costimulatory domain. 3zeta: CD3 zeta stimulatory domain. 2A: T2A ribosomal skip motif. tEGFR: truncated epidermal growth factor receptor.

(A) CD19 CAR with 4-1BB costimulatory domain. (SEQ ID NO: 1 to SEQ ID NO: 9)

(B) CD19 CAR with CD28 costimulatory domain. (SEQ ID NO: 10 to SEQ ID NO: 18)

(C) ROR1 CAR with 4-1BB costimulatory domain. (SEQ ID NO: 19 to SEQ ID NO: 29)

(D) SLAMF7 CAR with 4-1BB costimulatory domain (SEQ ID NO: 30 to SEQID NO: 40)

(E) SLAMF7 CAR with CD28 costimulatory domain (SEQ ID NO: 41 to SEQ ID NO: 51)

The amino acid sequences of (A) to (E) are represented in the one-letter amino acid code, in an N- to C-terminal order. Note that the C-terminal ends of the amino acid sequences are denoted by an asterisk.

FIG. 2: Dasatinib blocks the cytolytic activity of CD8⁺ CAR-T cells.

The cytolytic activity of CD8⁺ CAR-T cells was analyzed in a bioluminescence-based cytotoxicity assay in vitro. Diagram shows the cytolytic activity of CD8⁺ CAR-T cells in the absence of dasatinib (0 nM), and in the presence of titrated doses of dasatinib (12.5-100 nM). The percent specific lysis mediated by CAR-T cells was calculated using non-CAR modified T cells as reference and control. Specific lysis was determined at 1-hour intervals for up to 12 hours. Data shown are summary data obtained in independent experiments with CAR-T cell lines prepared from n=3 donors. * p<0.05, ** p<0.01, *** p<0.001.

A) Dasatinib blocks the cytolytic activity of CD8⁺ T cells expressing a CD19 CAR with 4-1BB costimulatory domain. Target cells in this assay: K562/CD19.

B) Dasatinib blocks the cytolytic activity of CD8⁺ T cells expressing a CD19 CAR with CD28 costimulatory domain. Target cells in this assay: K562/CD19.

C) Dasatinib blocks the cytolytic activity of CD8⁺ T cells expressing a ROR1 CAR with 4-1BB costimulatory domain. Target cells in this assay: K562/ROR1.

FIG. 3: Dasatinib blocks cytokine production and secretion in CD8. CAR-T cells.

CD8⁺ CAR-T cells were co-cultured with antigen-positive (K562/CD19 or K562/ROR1) target cells, either in the absence of dasatinib (0 nM) or in the presence of dasatinib (6.25-100 nM). The cytokines IFN-γ and IL-2 were measured by ELISA in supernatant obtained from these co-cultures after 20 hours of incubation. The amount of each cytokine that was produced specifically in response to antigen was determined subtracting the amount of each cytokine obtained without stimulation. Diagram shows the relative amount (in percent, normalized to the amount of cytokines released in the absence of dasatinib) of IFN-γ and IL-2 that was produced specifically in response to stimulation with antigen-positive target cells in the presence of dasatinib. Unless otherwise indicated, data shown are summary data obtained in independent experiments with CAR-T cell lines prepared from n=3 donors. * p<0.05, ** p<0.01.

A) Dasatinib blocks production and secretion of IFN-γ (left diagram) and IL-2 (right diagram) in CD8⁺ T cells expressing a CD19 CAR with 4-1BB costimulatory domain.

B) Dasatinib blocks production and secretion of IFN-γ (left diagram) and IL-2 (right diagram, n=2) in CD8⁺ T cells expressing a CD19 CAR with CD28 costimulatory domain.

C) Dasatinib blocks production and secretion of IFN-γ (left diagram) and IL-2 (right diagram) in CD8⁺ T cells expressing a ROR1 CAR with 4-1BB costimulatory domain.

FIG. 4: Dasatinib blocks proliferation of CD8⁺ CAR-T cells.

CD8⁺ CAR-T cells were labeled with CFSE and co-cultured with antigen-positive (K562/CD19 or K562/ROR1) target cells, either in the absence of dasatinib (0 nM) or in the presence of dasatinib (3.125-100 nM). The proliferation of CAR-T cells was analyzed by flow cytometry after 72 hours of incubation and the proliferation index determined. Diagram shows the relative proliferation (in percent, normalized to the proliferation index of CAR-T cells in the absence of Dasatinib) in response to stimulation with antigen-positive target cells in the presence of Dasatinib. Data shown are summary data obtained in independent experiments with CAR-T cell lines prepared from n=3 donors. * p<0.05, ** p<0.01.

A) Dasatinib blocks proliferation of CD8⁺ T cells expressing a CD19 CAR with 4-1BB costimulatory domain.

B) Dasatinib blocks proliferation of CD8⁺ T cells expressing a CD19 CAR with CD28 costimulatory domain.

C) Dasatinib blocks proliferation of CD8 T cells expressing a ROR1 CAR with 4-1BB costimulatory domain.

FIG. 5: Dasatinib blocks cytokine production and secretion in CD4⁺ CAR-T cells.

CD4⁺ CAR-T cells were co-cultured with antigen-positive (K562/CD19) target cells, either in the absence of dasatinib (0 nM) or in the presence of dasatinib (3.125-100 nM). The cytokines GM-CSF, IFN-γ, IL-2, IL-4, IL-5, IL-6 and IL-8 were measured by multiplex cytokine assay in supernatant obtained from these co-cultures after 20 hours of incubation. Diagram shows the amount of cytokines that was produced specifically in response to stimulation with antigen-positive target cells. Data shown are summary data obtained in independent experiments with CAR-T cell lines prepared from n=2 donors. * p<0.05, ** p<0.01, *** p<0.001.

A) Dasatinib blocks production and secretion of cytokines in CD4r T cells expressing a CD19 CAR with 4-1BB costimulatory domain.

B) Dasatinib blocks production and secretion of cytokines in CD4⁺ T cells expressing a CD19 CAR with CD28 costimulatory domain.

FIG. 6: Dasatinib blocks the function of SLAMF7 CAR-T cells

A) The cytolytic activity of CD8⁺ SLAMF7 CAR-T cells (upper diagram: with 4-1BB costimulatory domain; lower diagram: with CD28 costimulatory domain) was analyzed in a bioluminescence-based cytotoxicity assay in vitro. Diagrams show the cytolytic activity of CD8⁺ CAR-T cells against K562/SLAMF7 in the absence of dasatinib (0 nM), and in the presence of titrated doses of dasatinib (20-100 nM). The percent specific lysis mediated by CAR-T cells was calculated using non-CAR modified T cells as reference and control. Specific lysis was determined at 1-hour intervals for up to 14 hours. Data shown are summary data obtained in independent experiments with CAR-T cell lines prepared from n=2 donors.

B) CD8⁺ SLAMF7 CAR-T cells (light grey: with 4-1BB costimulatory domain; dark grey: with CD28 costimulatory domain) were co-cultured with antigen-positive (K562/SLAMF7) target cells, either in the absence of dasatinib (0 nM) or in the presence of dasatinib (20-100 nM). The cytokines IFN-γ (left diagram) and IL-2 (right diagram) were measured by ELISA in supernatant obtained from these co-cultures after 20 hours of incubation. The amount of each cytokine that was produced specifically in response to antigen was determined subtracting the amount of each cytokine obtained without stimulation. Diagram shows the relative amount (in percent, normalized to the amount of cytokines released in the absence of dasatinib) of IFN-γ and IL-2 that was produced specifically in response to stimulation with antigen-positive target cells in the presence of dasatinib. Data shown are summary data obtained in independent experiments with CAR-T cell lines prepared from n=2 donors. *** p<0.001.

C) CD4⁺ SLAMF7 CAR-T cells (light grey: with 4-1BB costimulatory domain; dark grey: with CD28 costimulatory domain) were co-cultured with antigen-positive (K562/SLAMF7) target cells, either in the absence of dasatinib (0 nM) or in the presence of dasatinib (20-100 nM). The cytokines IFN-γ (left diagram) and IL-2 (right diagram) were measured by ELISA in supernatant obtained from these co-cultures after 20 hours of incubation. The amount of each cytokine that was produced specifically in response to antigen was determined subtracting the amount of each cytokine obtained without stimulation. Diagram shows the relative amount (in percent, normalized to the amount of cytokines released in the absence of dasatinib) of IFN-γ and IL-2 that was produced specifically in response to stimulation with antigen-positive target cells in the presence of dasatinib. Data shown are summary data obtained in independent experiments with CAR-T cell lines prepared from n=2 donors. *** p<0.001,

FIG. 7: Dasatinib blocks the phosphorylation of tyrosine kinases involved in CAR-signaling.

CD8⁺ T cells expressing a CD19 CAR with 4-1BB costimulation were co-cultured with RCH-ACV target cells either in the absence of dasatinib (dasatinib −) or in the presence of dasatinib (100 nM; dasatinib +). Western blots were performed to determine phosphorylation and total protein expression of Lck/Src family kinases (Y416), CAR-associated CD3zeta (Y142), ZAP70 (Y319).

A) Western blots showing phosphorylation of Src family kinase (Y416), CAR-associated CD3zeta (Y142), ZAP70 (Y319), and the total expression of the corresponding proteins Lck, CD3zeta and ZAP70 in dasatinib-treated vs. dasatinib-untreated T cells. β-actin is stained as a loading control und used for normalization.

B) Diagram shows relative phosphorylation (as percent) in dasatinib-untreated T cells (100%) vs. dasatinib-treated T cells. Summary data obtained by quantitative Western blot analyses in n=3 independent experiments. * p<0.05.

FIG. 8: Dasatinib blocks NFAT mediated expression of GFP in CD8⁺ and CD4⁺ CAR-T cells.

CD8⁺ (left panel) and CD4⁺ (right panel) T cells expressing a CD19 CAR with 4-1BB costimulation were modified with an NFAT-inducible GFP-reporter gene. T cells were then stimulated with CD19-positive (Raji) or CD19-negative (K562) target cells, either in the presence of dasatinib (100 nM; dasatinib +) or the absence of dasatinib (dasatinib −) for 24 hours, and the reporter gene induction was analyzed by flow cytometry. Diagrams show the mean fluorescence intensity (MFI) obtained for GFP (green fluorescent protein) in the FITC channel. Results show summary data obtained in n=3 independent experiments. ** p<0.01, *** p<0.001.

FIG. 9: Blockade with dasatinib does not decrease the viability of CAR-T cells CD8⁺ T cells expressing a CD19 CAR with 4-1BB costimulation were co-cultured with CD19-positive target cells (K562/CD19) for 24 hours, either in the absence of dasatinib (dasatinib −) or in the presence of dasatinib (100 nM, dasatinib +). In one setting, dasatinib was added to the medium at 1 hour after the start of the co-culture [dasatinib (+)]. At the end of the co-culture, the percentage of alive T cells (Annexin-V⁻/7-AAD⁻), T cells in apoptosis (Annexin-V⁺/7-AAD⁻), and dead T cells (Annexin-V⁺/7-AAD⁺) was determined by flow cytometry. Diagram shows the mean percentage of alive, apoptotic and dead T cells obtained in n=3 independent experiments. * p<0.05.

FIG. 10: Dasatinib blocks the function of activated CD8⁺ CAR-T cells.

A) Dasatinib blocks the cytolytic activity of activated CD8⁺ CAR-T cells expressing a CD19 CAR with 4-18B costimulatory domain. The cytolytic activity of CD8⁺ CAR-T cells was analyzed in a bioluminescence-based cytotoxicity assay in vitro as shown in FIG. 2. Dasatinib (100 nM) was either added at the start of the cytotoxicity assay (0 h) or 1 hour after the start of the cytotoxicity assay (1 h). Results show summary data obtained in n=3 independent experiments. * p<0.05, ** p<0.01, *** p<0.001.

B) Dasatinib blocks cytokine production and secretion of activated CD8⁺ CAR-T cells. The cytokine production and secretion was analyzed by ELISA as shown in FIG. 3. Dasatinib (100 nM) was either added at the start of the co-culture (0 h) or 2 hours after the start of the co-culture (+2 h). Results show summary data obtained in n=3 independent experiments.* p<0.05, ** p<0.01, *** p<0.001.

C) Dasatinib blocks the proliferation of activated CD8⁺ CAR-T cells. Proliferation was analyzed by CFSE dye dilution as shown in FIG. 4. Dasatinib (100 nM) was either added at the start of the co-culture (0 h), or 1 hour (+1 h), 3 hours (+3 h) or 48 hours (+48 h) after the start of the co-culture. Results show summary data obtained in n=3 independent experiments. * p<0.05, *** p<0.001.

FIG. 11: Dasatinib prevents CAR-T cell activation during sequential stimulation CD8⁺ (left panel) and CD4⁺ (right panel) T cells expressing a CD19 CAR with 4-1BB costimulation were modified with an NFAT-inducible GFP-reporter gene. T cells were then stimulated with CD19-positive (Raji) target cells every 24 hours. Dasatinib was added either at assay start (black circles) or one hour after assay start (dasa +1 h, grey circles), and was then added to the medium every 24 hours simultaneously with new target cells. Untreated CAR T cells were included for comparison (untreated, white circles). Reporter gene induction was analyzed by flow cytometry. Diagrams show the mean fluorescence intensity (MFI) obtained for GFP (green fluorescent protein) in the FITC channel. Data shown are mean values+SD obtained in n=2 (CD8⁺) and n=3 experiments (CD4⁺) with T cells from different healthy donors. * P≤0.05, ** P≤0.01, *** P≤0.001 by two way ANOVA.

FIG. 12: The blockade of CAR-T cell function is rapidly and completely reversible after short-term exposure to dasatinib.

The blockade of CAR-T cell cytolytic activity is rapidly and completely reversible after short-term, 2-hour exposure to dasatinib. The cytolytic activity of CD8⁺ CAR-T cells was analyzed in a bioluminescence-based cytotoxicity assay in vitro as shown in FIG. 2. Dasatinib (100 nM) was added at the start of the cytotoxicity assay (−2 h) and then washed away (0 h). CD8⁺ CAR-T cells that were not exposed to dasatinib (0 nM) served as a reference. ***p<0.001.

A) Assay performed with CD8⁺ T cells expressing a CD19 CAR with 4-1BB costimulation. Data shown are summary data obtained in n=3 independent experiments.

B) Assay performed with CD8⁺ T cells expressing a CD19 CAR with CD28 costimulation. Data shown are summary data obtained in n=2 independent experiments.

FIG. 13: Long-term exposure to dasatinib does not decrease the viability of CAR-T cells.

CD8⁺ T cells expressing a CD19 CAR with 4-1BB costimulation were maintained in culture medium that contained dasatinib [100 nM, (+)]. Before co-culture [d0(−)], after 2 days (d2) and after 8 days (d8) the percentage of alive T cells (Annexin-V⁻/7-AAD), T cells in apoptosis (Annexin-V⁺/7-AAD⁻), and dead T cells (Annexin-V⁺/7-AAD⁺) was determined by flow cytometry. Untreated CD8⁺ CAR-T cells [(−)] were stained for comparison at the referring days. Diagram shows the mean percentage of alive, apoptotic and dead T cells obtained in data from one healthy donor.

FIG. 14: The blockade of CAR-T cell function is rapidly and completely reversible after long-term exposure to and subsequent removal of dasatinib; the blockade of CAR-T cell function is still effective after long-term exposure to dasatinib.

A) The blockade of CAR-T cell cytolytic activity is rapidly and completely reversible after long-term exposure to and subsequent removal of dasatinib; the blockade of CAR-T cell cytolytic activity is still effective after long-term exposure to dasatinib.

CD8⁺ T cells expressing a CD19 CAR with 4-1BB costimulation were maintained in culture medium that contained dasatinib (100 nM). After 1 day (left panel) and after 7 days (right panel), an aliquot of CD8⁺ CAR-T cells was washed, and their cytolytic activity analyzed in a bioluminescence-based cytotoxicity assay in vitro as shown in FIG. 2. To analyze whether the blockade of cytolytic activity was still effective after long-term exposure to dasatinib, dasatinib was added to the co-culture to a final concentration of 100 nM at the beginning of the cytotoxicity assay. Data shown are summary data obtained in independent experiments with CAR-T cell lines prepared from n=3 donors.

Key to legend: no dasa/no dasa: not exposed to dasatinib during 1-day or 7-day culture, and dasatinib not present during the cytotoxicity assay. no dasa/dasa: not exposed to dasatinib during 1-day or 7-day culture, dasatinib present during the cytotoxicity assay. dasa/no dasa: Exposed to dasatinib during 1-day or 7-day culture, and dasatinib not present during cytotoxicity assay. dasa/dasa: Exposed to dasatinib during 1-day or 7-day culture, and dasatinib present during the cytotoxicity assay. *** p<0.001.

B) The blockade of CAR-T cell cytokine production and secretion is rapidly and completely reversible after long-term exposure to and subsequent removal of dasatinib; the blockade of CAR-T cell cytokine production and secretion is still effective after long-term exposure to dasatinib.

CD8⁺ T cells expressing a CD19 CAR with 4-1BB costimulation were maintained in culture medium that contained dasatinib (100 nM). After 1 day and after 7 days, an aliquot of CD8 CAR-T cells was washed, and cytokine production and secretion analyzed as shown in FIG. 3. To analyze whether the blockade of cytokine production and secretion was still effective after long-term exposure to dasatinib, dasatinib was added at the beginning of co-culture to a final concentration of 100 nM. Data shown are summary data obtained in independent experiments with CAR-T cell lines prepared from n=3 donors.

Key to legend: Dasa pre −: not exposed to dasatinib during 1-day or 7-day culture. Dasa pre 1: exposed to dasatinib for 1 day. Dasa pre 7: exposed to dasatinib for 7 days. Dasa during −: dasatinib not present during co-culture for cytokine assay. Dasa during +: dasatinib present during co-culture for cytokine assay. *** p<0.001.

C) The blockade of CAR-T cell proliferation is rapidly and completely reversible after long-term exposure to and subsequent removal of dasatinib; the blockade of CAR-T cell proliferation is still effective after long-term exposure to dasatinib.

CD8⁺ T cells expressing a CD19 CAR with 4-1BB costimulation were maintained in culture medium that contained dasatinib (100 nM). After 1 day and after 7 days, an aliquot of CD8⁺ CAR-T cells was washed, and proliferation analyzed as shown in FIG. 4. To analyze whether the blockade of proliferation was still effective after long-term exposure to dasatinib, dasatinib was added at the beginning of co-culture to a final concentration of 100 nM. Data shown are summary data obtained in independent experiments with CAR-T cell lines prepared from n=3 donors.

Key to legend: Dasa pre −: not exposed to dasatinib during 1-day or 7-day culture. Dasa pre 1: exposed to dasatinib for 1 day. Dasa pre 7: exposed to dasatinib for 7 days. Dasa during−: dasatinib not present during co-culture for proliferation assay. Dasa during +: dasatinib present during co-culture for proliferation assay. ** p<0.01, *** p<0.001.

FIG. 15: Dasatinib blocks cytokine secretion from CAR-T cells in vivo and prevents cytokine release syndrome.

A) Experiment setup and treatment schedule: NSG mice were inoculated with firefly-luciferase-transduced Raji tumor cells by i.v. tail vein injection on day −7; dasatinib was administered by i.p. injection every 6 hours from day 0 at 0 hours until day 1 at 30 hours (total 6 doses). CAR-T cells (i.e. CD8⁺ and CD4⁺ CD19 CAR/4-1BB T cells, total dose: 5×10e6; CD8:CD4 ratio=1:1) or control untransduced T cells were administered on day 0 at 3 hours. Bioluminescence imaging was performed on day −1, on day 1 and on day 3 to determine tumor burden. On day 1 at 33 hours, and on day 3, cohorts of mice were sacrificed and peripheral blood (PB), bone marrow (BM) and spleen (SP) analyzed.

B) Cytokine levels in mouse serum were determined by multiplex cytokine analysis in samples obtained on day 1 at 33 hours and on day 3. Diagrams show the concentration of GM-CSF, IFN-γ, TNF-α, IL-2, IL-5 and IL-6, respectively, obtained in cohorts of mice that had been treated with: untransduced control T cells and received no dasatinib (ctrl/−); CD19 CAR-T cells and received no dasatinib (CAR/−); CD19 CAR-T cells and received dasatinib (CAR/+). * p<0.05; ** p<0.01.

C) Raji tumor burden was determined by bioluminescence imaging on day −1, on day 1 and on day 3. Diagram shows the mean fold-change in bioluminescence signal between day −1 and day 1 (black bars), and day 1 and day 3 (grey bars); obtained in cohorts of mice that had been treated with: untransduced control T cells and received no dasatinib (ctrl/−); untransduced control T cells and received dasatinib (ctrl/+); CD19 CAR-T cells and received no dasatinib (CAR/−); CD19 CAR-T cells and received dasatinib (CAR/+). ** p<0.01; *** p<0.001.

D) The presence of adoptively transferred CAR-modified and control untransduced T cells in peripheral blood (PB), bone marrow (BM) and spleen (Sp) was analyzed by flow cytometry on day 1 and day 3. The diagram shows the frequency of CAR-modified and control untransduced T cells (identified as human CD3⁺/human CD45⁺) as percentage of live (7-AAD⁻) cells.

Key to legend: control/untreated: mice had received untransduced control T cells and received no dasatinib; control/treated: mice had received untransduced control T cells and received dasatinib; CAR/untreated: mice had received CD19 CAR-T cells and received no dasatinib; CAR/treated: mice had received CD19 CAR-T cells and had received dasatinib.

E) The adoptively transferred CD19 CAR/4-1BB-modified and untransduced control T cells had also been equipped with the NFAT-inducible GFP-reporter gene. The expression of the GFP-reporter gene was analyzed in CAR-modified and control T cells in bone marrow (bottom diagram) and spleen (top diagram) by flow cytometry. The diagram shows the mean fluorescence intensity (MFI) of GFP in CAR-modified and control untransduced T cells (identified as human CD3⁺/human CD45⁺).

Key to legend: control/untreated: mice had received untransduced control T cells and received no dasatinib; control/treated: mice had received untransduced control T cells and received dasatinib; CAR/untreated: mice had received CD19 CAR-T cells and received no dasatinib; CAR/treated: mice had received CD19 CAR-T cells and had received dasatinib.

*p<0.05; ** p<0.01; *** p<0.001.

FIG. 16: Dasatinib pauses activated CD19 CAR/4-1BB-T cells in a function OFF state in vivo

A) Experiment setup and treatment schedule: NSG mice were inoculated with firefly-luciferase-transduced Raji tumor cells by Lv. tail vein injection on day 0. CAR-T cells (i.e. CD8⁺ and CD4⁺ CD19 CAR/4-1BB T cells, total dose: 5×10e6; CD8:CD4 ratio=1:1) or control untransduced T cells were administered on day 7. Dasatinib was administered every 6 hours between day 10 and day 12 (total 8 doses) to create a function ON OFF ON sequence. Bioluminescence imaging and bleeding was performed on day 7, 10, 12, 14, 17 and bioluminescence imaging was continued subsequently once weekly (dx) to determine tumor burden.

B) Development of tumor burden measured as ventral average luminescence over time. Upper diagram shows development of individual mice, lower diagram shows mean BLI of each treatment cohort. Key to Legend: ctrl (ON/OFF/ON): mice had received untransduced control T cells and dasatinib between day 10 and day 12; CAR (ON):mice had received CD19 CAR-T cells and no dasatinib; CAR (ON/OFF/ON): mice had received CD19 CAR-T cells and dasatinib between day 10 and day 12.

C) Diagrams show the relative change in tumor burden between indicated days; obtained in cohorts of mice that had been treated with: untransduced control T cells and dasatinib (ctrl (ON/OFF/ON)); CD19 CAR-T cells and no dasatinib (CAR (ON)); CD19 CAR-T cells and dasatinib (CAR (ON/OFF/ON). ** p<0.01; *** p<0.001.

D) Cytokine levels in mouse serum were determined by multiplex cytokine analysis in samples obtained on day 10, day 12, day 14 and day 17. Diagrams show the concentration of IFN-γ: left diagram shows the mean IFNγ and individual data points. Right diagram displays the development of each mouse in each treatment cohort. * p<0.05; ** p<0.01.

Key to Legend: ctrl (ON/OFF/ON): mice had received untransduced control T cells and dasatinib between day 10 and day 12; CAR (ON):mice had received CD19 CAR-T cells and no dasatinib; CAR (ON/OFF/ON): mice had received CD19 CAR-T cells and dasatinib between day 10 and day 12.

FIG. 17: Dasatinib pauses activated CD19 CAR/CD28-T cells in a function OFF state in vivo

A) Experiment setup and treatment schedule: NSG mice were inoculated with firefly-luciferase-transduced Raji tumor cells by Lv. tail vein injection on day 0. CAR-T cells (i.e. CD8⁺ and CD4⁺CD19 CAR/CD28 T cells, total dose: 5×10e6; CD8:CD4 ratio=1:1) or control untransduced T cells were administered on day 7. Dasatinib was administered every 6 hours between day 10 and day 12 (total 8 doses) to create a function ON OFF ON sequence. Bioluminescence imaging and bleeding was performed on day 7, 10, 12, 14, 17, and bioluminescence imaging was continued subsequently once weekly (dx) to determine tumor burden.

B) Development of tumor burden measured as ventral average luminescence over time. Left diagram shows median BLI of each treatment cohort; right diagram shows development of individual mice.

Key to Legend: CAR/Dasa: mice had received CD19/CD28 CAR-T cells and dasatinib between day 10 and day 12; CAR/DMSO:mice had received CD19 CAR-T cells and no dasatinib but injections with vehicle between day 10 and day 12; ctrl/Dasa: mice had received untransduced control T cells and dasatinib between day 10 and day 12; CAR/−: mice had received CD19 CAR-T cells no injections.

C) Diagrams show the relative change in tumor burden between indicated days; obtained in cohorts of mice that had been treated according to the legend.

Key to legend: ctrl/Dasa: mice had received untransduced control T cells and dasatinib between day 10 and day 12; CAR/Dasa: mice had received CD19/CD28 CAR-T cells and dasatinib between day 10 and day 12; CAR/DMSO:mice had received CD19 CAR-T cells and no dasatinib but injections with vehicle between day 10 and day 12; CAR/−: mice had received CD19 CAR-T cells no injections. ** p<0.01; *** p<0.001.

FIG. 18: Dasatinib exerts superior control over CAR-T cell function compared to dexamethasone.

A) Dasatinib exerts superior control over cytolytic activity by CAR-T cells compared to dexamethasone

The cytolytic activity of CD8⁺ T cells expressing a CD19 CAR with 4-1BB costimulation was analyzed in a bioluminescence-based cytotoxicity assay in vitro. Diagram shows the cytolytic activity of CD8⁺ CAR-T cells in the absence of dexamethasone (0 M), and in the presence of titrated doses of dexamethasone (0.1-100 μM) (top diagram). In some experiments, T cells were pre-treated with dexamethasone at the indicated dose for 24 hours and the cytotoxicity assay performed as described above (bottom diagram). Cytolytic activity of CAR-T cells in the presence of 0.1 μM dasatinib is shown as a reference and for comparison. The percent specific lysis mediated by CAR-T cells was calculated using unspecific control T cells and was determined at 1-hour intervals for up to 10 hours. Data shown are summary data obtained in independent experiments with CAR-T cell lines prepared from n=3 donors. * p<0.05, *** p<0.001.

B) Dasatinib exerts superior control over cytokine production and secretion in CAR-T cells compared to dexamethasone

CD8⁺ CAR-T cells were co-cultured with antigen-positive (K562/CD19) target cells, either in the absence of dexamethasone (0 μM) or in the presence of dexamethasone (0.1-100 μM). The cytokines IFN-γ and IL-2 were measured by ELISA in supernatant obtained from these co-cultures after 20 hours of incubation. The amount of each cytokine that was produced specifically in response to antigen was determined by subtracting the amount obtained without stimulation from the amount obtained after stimulation with K562/CD19 antigen-positive target cells. Diagrams show the relative amount (in percent, normalized to the amount of cytokines released in the absence of treatment) of IFN-γ (top diagram, grey bars) and IL-2 (bottom diagram, grey bars) that was produced specifically in response to stimulation with antigen-positive target cells. In some experiments, T cells were pre-treated with dexamethasone at the indicated dose for 24 hours (black bars). The cytokine production of CAR-T cells in the presence of 0.1 μM dasatinib is shown as a reference and for comparison. Data shown are summary data obtained in independent experiments with CAR-T cell lines prepared from n=3 donors. * p<0.05, ** p<0.01.

C) Dasatinib exerts superior control over proliferation of CAR-T cells compared to dexamethasone regarding the proliferation of CD8⁺ CAR-T cells

CD8⁺ CAR-T cells were labeled with CFSE and co-cultured with antigen-positive (K562/CD19) target cells, either in the absence of dexamethasone (0 μM) or in the presence of dexamethasone (0.1-100 μM). The proliferation of CAR-T cells was analyzed by flow cytometry after 72 hours of incubation and the proliferation index determined. Diagram shows the relative proliferation (in percent, normalized to the proliferation index of CAR-T cells in the absence of treatment) in response to stimulation with antigen-positive target cells (grey bars). In some experiments, T cells were pre-treated with dexamethasone at the indicated dose for 24 hours (black bars). The proliferation of CAR-T cells in the presence of 0.1 M dasatinib is shown as a reference and for comparison. Data shown are summary data obtained in independent experiments with CAR-T cell lines prepared from n=3 donors. *** p<0.001.

FIG. 19: The influence of dasatinib and other clinically approved tyrosine-kinase inhibitors on the function of CAR-T cells.

A) Cytolytic activity: The cytolytic activity of CD8⁺ T cells expressing a ROR1 CAR with 4-1BB costimulatory domain was analyzed in a bioluminescence-based cytotoxicity assay. Diagram shows the cytolytic activity in the presence of 100 nM dasatinib, 5.3 μM imatinib, 4.2 μM lapatinib and 3.6 μM nilotinib, or untreated as control. The percent specific lysis of antigen positive target cells (RCH-ACV) mediated by CAR-T cells was calculated using unspecific control T cells as a reference and was determined at 1-hour intervals for up to 8 hours. Data shown are summary data obtained in independent experiments with CAR-T cell lines prepared from n=2 donors.

B) IFN-γ production: CD8⁺ CAR-T cells expressing a ROR1 CAR with 4-1BB costimulatory domain were co-cultured with antigen-positive (RCH-ACV) target cells in the presence of 100 nM dasatinib, 5.3 μM imatinib, 4.2 μM lapatinib and 3.6 μM nilotinib, or untreated as control. IFN-γ was measured by ELISA in supernatant obtained from these co-cultures after 20 hours of incubation. The amount of IFN-γ that was produced specifically in response to antigen was determined by subtracting the amount obtained without stimulation from the amount obtained with antigen-positive target cells. Data shown are summary data obtained in independent experiments with CAR-T cell lines prepared from n=2 donors.

C) Proliferation: CD8⁺ T cells expressing a ROR1 CAR with 4-1BB costimulatory domain were labeled with CFSE and co-cultured with antigen-positive (RCH-ACV) target cells, in the presence of 100 nM dasatinib, 5.3 μM imatinib, 4.2 μM lapatinib and 3.6 μM nilotinib, or untreated as control. The proliferation of CAR-T cells was analyzed by flow cytometry after 72 hours of incubation. The table below the histogram provides the percentage of CAR-T cells that underwent >3/2/1 cell divisions, respectively.

FIG. 20: The influence of dasatinib and other Src-kinase inhibitors on the cytolytic activity of CAR-T cells.

The cytolytic activity of CD8⁺ T cells expressing a CD19 CAR with 4-1BB costimulatory domain was analyzed in a bioluminescence-based cytotoxicity assay. Diagram shows the cytolytic activity of CD8⁺ CAR-T cells in the presence of titrated doses (1-1000 nM) of saracatinib, bosutinib, PP1-inhibitor or dasatinib. The percent specific lysis of antigen-positive target cells (K562/CD19) compared to untransduced control T cells was determined after 4 hours of co-culture.

FIG. 21: Intermittent exposure to dasatinib augments the antitumor function of CAR-T cells in vivo.

A) Experiment setup and treatment schedule: NSG mice were inoculated with firefly-luciferase-transduced Raji tumor cells by i.v. tail vein injection on day 0. CAR-T cells (i.e. CD8 and CD4 T cells expressing a CD19 CAR with 4-1BB costimulatory domain, total dose: 5×10e6; CD8:CD4 ratio=1:1) or control untransduced T cells were administered on day 7 by i.v. tail vein injection. Dasatinib was administered by i.p. injection every 24 hours from d7 until d11 followed by i.p. injection every 36 hours on d12 and 14 (total 7 doses). Serial bioluminescence imaging was performed to determine tumor burden. On day 15, mice were sacrificed and peripheral blood (PB), bone marrow (BM) and spleen (SP) analyzed.

B) Tumor burden assessed by bioluminescence imaging. Diagram shows the dorsal bioluminescence signal as average radiance in p/s/cm²/sr obtained from regions of interest encompassing the entire dorsal body of each mouse in the respective treatment cohort. Each cohort consists of two animals.

Key to legend: ctrl/−: mice had received untransduced control T cells and received no dasatinib; ctrl/+: mice had received untransduced control T cells and received dasatinib; CAR/−: mice had received CD19 CAR-T cells and received no dasatinib; CAR/+: mice had received CD19 CAR-T cells and had received dasatinib.

FIG. 22: Intermittent exposure to dasatinib augments the engraftment, proliferation and persistence of CAR-T cells in vivo.

Experiment setup and treatment schedule is same as in FIG. 21A. The presence of adoptively transferred CD19 CAR-modified and control untransduced T cells in peripheral blood (PB), bone marrow (BM) and spleen (SP) was analyzed by flow cytometry.

A) Gating strategy and data obtained in exemplary mice from the treatment cohort that received CD19 CAR-T cells but not dasatinib (CD19 CAR, upper panel), and the treatment cohort that received CD19 CAR-T cells and dasatinib (lower panel).

B) The diagram shows the frequency of CAR-modified and control untransduced T cells (identified as human CD3⁺/human CD45⁺) as percentage of live (7-AAD) cells. Each cohort consists of two animals.

Key to legend: ctrl/−: mice had received untransduced control T cells and received no dasatinib; ctrl/+: mice had received untransduced control T cells and received dasatinib; CAR/−: mice had received CD19 CAR-T cells and received no dasatinib; CAR/+: mice had received CD19 CAR-T cells and had received dasatinib.

FIG. 23: Intermittent treatment with dasatinib decreases expression of PD1 on CAR-T cells.

Experiment setup and treatment schedule is same as in FIG. 21A.

The diagram shows expression of PD-1 on CD19 CAR/4-1BB T cells as mean fluorescence intensity (MDI) obtained after staining with anti-PD1 mAb. Each cohort consists of 4 animals.** p<0.01; *** p<0.001.

Key to legend: CAR/−: mice had received CD19 CAR-T cells and received no dasatinib (black bars); CAR/+: mice had received CD19 CAR-T cells and had received dasatinib (grey bars).

FIG. 24: CAR-T cells that are blocked by dasatinib are susceptible to subsequent elimination with the iCasp9 suicide gene.

CD8 T cells expressing a CD19 CAR with 4-1BB costimulation were modified with an iCapase9 suicide gene. T cells were cultured in medium supplemented with 50 U/ml IL-2, in the absence or presence of 100 nM dasatinib, and in the absence or presence of an iCaspase inducer drug. After 24 hours, cells were labeled with anti-CD3 mAB and analyzed by flow cytometry for the presence of iCasp+ T cells.

The diagram shows the percentage of iCasp+ cells as percentage of CAR-T cells.

### DETAILED DESCRIPTION OF THE INVENTION

Adoptive immunotherapy with gene-modified CAR-T cells is a rapidly evolving translational research field in medicine. CD19-specific CAR-T cells have been demonstrated to induce durable complete remissions in end-stage leukemia and lymphoma patients [2], [7], [10], [19], [20]. Major concerns associated with CAR-T cell therapy relate to the occurrence of acute and chronic, potentially life-threatening side effects; and the inability to control the function and fate of these engineered T cells once they have been infused into the patient.

Current strategies for treating side effects of CAR-T cell therapy include attempts to neutralize cytokines like IL-6 that have been associated with the clinical occurrence of CRS; the use of steroids to reduce the activity of CAR-T cells, and the incorporation of suicide genes and depletion markers to eliminate CAR-T cells. All of these strategies have major shortcomings: attempts to neutralize or prevent the binding if cytokines to their receptors is a symptomatic treatment that does not exert a direct effect on the CAR-T cells themselves; steroids exert only incomplete control over CAR-T cells and are unable to prevent or stop CRS and other side effects; suicide genes and depletion markers aim at eliminating CAR-T cells and also terminate the antitumor effect, which is not desired by patient and physician. None of the currently known strategies allows patient or treating physician to exert precise, on-time control over the function of CAR-T cells in the patient's body.

According to the invention, dasatinib is used to control the function of CAR-T cells in the patient's body, and enables patient and physician to exert precise, on-time ‘remote-control’ over CAR-T cells after their infusion.

According to the invention, dasatinib exerts a dose-dependent, titratable inhibitory effect on CAR-signaling and ensuing CAR-T cell function. Depending on the dose of dasatinib, the function of CAR-T cells can be partially or completely blocked. The dasatinib-induced blockade of CAR-T cell function can be exploited to mitigate or prevent toxicity, and control the function of CAR-T cells in the patient's body (see Example 2).

According to the invention, the functional blockade of CAR-T cells by dasatinib has a rapid, immediate onset. The blockade is complete if dasatinib is provided at a concentration above a certain threshold (i.e. there is complete inhibition of cytolytic activity, cytokine secretion and proliferation of CAR-T cells). Below this threshold, dasatinib exerts a partial blockade of CAR-T cell functions. The mechanism of dasatinib-induced CAR-T cell inhibition/blockade includes but is not limited to the blockade of CAR-signaling through interference with phosphorylation of endogenous Src-kinases like Lck, and interference with the formation and function of transcription factors like NFAT (see Example 2).

According to the invention, dasatinib is able to inhibit and block CAR-T cell function in both CD8⁺ and CD4⁺ T cells, and is universally applicable to any synthetic receptor construct that uses, at least in part, signaling through endogenous Src-kinases like Lck, and transcription factors like NFAT (see Example 2).

The present invention does not only enable preventing the activation of resting non-activated CAR-T cells, but also blocks the function of CAR-T cells that are already activated and in the process of exerting their effector functions (see Example 3). This is of particular importance given that at clinical diagnosis of CRS and clinical manifestations of side effects, CAR-T cells in the patient's body are already activated.

According to the invention, the blocking effect of dasatinib on CAR-T cell function is rapidly and completely reversible (see Example 5). The exposure of CAR-T cells to dasatinib does neither reduce their viability, nor compromises their ability to subsequently resume their antitumor function (see Example 5). This is a critically important and distinguishing feature from steroids (that reduce CAR-T cell viability and compromise their subsequent function) (see Example 9) and suicide genes/depletion markers that terminate CAR-T cells (see Example 13). According to the invention, dasatinib exerts complete control over all CAR-T cell functions (i.e. cytolytic activity, cytokine secretion including IFN-γ and IL-2, proliferation), whereas steroids only interfere with IL-2 secretion and proliferation, but do not inhibit cytolysis or secretion of IFN-γ (see Example 2 and 9).

According to the invention, the blocking effect of dasatinib on CAR-T cell is effective as long as the concentration of dasatinib is maintained above a certain threshold, and can be extended and perpetuated as desired by the patient or treating physician (see Example 5).

According to the invention, dasatinib can be used to prevent, mitigate or treat side effects that occur during or after CAR-T cell therapy. In particular, dasatinib can be used to mitigate, prevent and/or treat cytokine release syndrome (see Example 6).

According to the invention, dasatinib can be administered in any way suitable to achieve the desired concentration (e.g. serum level) in the patient's body. As non-limiting examples, this includes the use of any kind of pumps, infusion, injection and/or oral administration.

According to the invention, inhibition and/or blockade of CAR-T cell function may also be accomplished with other compounds that interfere with endogenous Src-kinases like Lck, and transcription factors like NFAT (see Example 10).

According to the invention, dasatinib can also be used to augment the antitumor function of CAR-T cells. As shown in Example 11, the intermittent exposure to dasatinib leads to increased viability of CAR-T cells after encountering tumor cells. Further, the intermittent exposure of CAR-T cells to dasatinib leads to superior engraftment, proliferation and persistence after adoptive transfer in vivo. Further, the intermittent exposure of CAR-T cells to dasatinib leads to superior antitumor function in vivo.

According to the invention, dasatinib can also be used to decrease the expression of check-point molecules on CAR-T cells, including but not limited to PD-1 (see Example 12). Therefore, the present invention also comprises the use of dasatinib to augment the antitumor function of CAR-T cells.

The finding that dasatinib is able to interfere with and completely block the function of CAR-T cells was unexpected and not foreseeable. CARs are synthetic designer receptors that comprise amino acid sequences and domains of proteins that occur in non-gene modified human T cells. However, these amino acid sequences and domains are combined in a new and artificial way, and there is at present no or only very limited knowledge on how these domains work in the CAR and generate/transmit their signal.

The finding that dasatinib is able to augment the function of CAR-T cells and decrease expression of PD1 on CAR-T cells after intermittent exposure to dasatinib was unexpected and not foreseeable. Rather, one would have expected that exposure of CAR-T cells to dasatinib has either no effect or exerts a toxic effect.

### Definitions and Embodiments

Unless otherwise defined below, the terms used in the present invention shall be understood in accordance with the common meaning known to the person skilled in the art.

Each publication, patent application, patent, and other reference cited herein is incorporated by reference in its entirety for all purposes to the extent that it is not inconsistent with the present invention. References are indicated by their reference numbers in square brackets and their corresponding reference details which are provided in the “references” section.

A “kinase inhibitor” as referred to herein is a molecular compound which inhibits one or more kinase(s) by binding to said kinase(s) and exerting an inhibiting effect on said kinase. A kinase inhibitor is capable of binding to one or more kinase species, upon which the kinase activity of the one or more kinase is reduced. A kinase inhibitor as referred to herein is typically a small molecule, wherein a small molecule is a molecular compound of low molecular weight (typically less than 1 kDa) and size (a diameter which is typically smaller than 1 nm).

In one embodiment, the kinase inhibitor is a tyrosine kinase inhibitor. In a preferred embodiment, the kinase inhibitor is a Src kinase inhibitor. In a more preferred embodiment, the kinase inhibitor is an Lck inhibitor. In a very preferred embodiment, the kinase inhibitor is dasatinib.

The terms “K_{D}” or “K_{D} value” relate to the equilibrium dissociation constant as known in the art. In the context of the present invention, these terms can relate to the equilibrium dissociation constant of a targeting agent (e.g. a CAR T-cell) with respect to a particular antigen of interest (e.g. CD19, ROR1, BCMA, or FLT3). The equilibrium dissociation constant is a measure of the propensity of a complex (e.g. an antigen-targeting agent complex) to reversibly dissociate into its components (e.g. the antigen and the targeting agent). Methods to determine K_{D} values are known in art.

It is to be understood that terms such as “a tyrosine kinase inhibitor” refer to the presence of a kinase inhibitor but do not exclude the possibility that additional kinase inhibitors, e.g. one, two, three or more additional kinase inhibitors could be present. In one embodiment in accordance with the invention, only one kinase inhibitor is used.

In one embodiment, the chimeric antigen receptor is capable of binding to an antigen, preferably a cancer antigen, more preferably a cancer cell surface antigen. In a preferred embodiment, the chimeric antigen receptor is capable of binding to extracellular domain of a cancer antigen.

In a preferred embodiment, the chimeric antigen receptor is expressed in immune cells, preferably T cells. In a preferred embodiment of the invention, the chimeric antigen receptor is expressed in T cells and allows said T cells to bind specifically to antigen-expressing cancer cells with high specificity to exert a growth inhibiting effect, preferably a cytotoxic effect, on said cancer cells.

“Adoptive immunotherapy” as described herein refers to the transfer of immune cells into a patient for targeted treatment of cancer. The cells may have originated from the patient or from another individual. In adoptive immunotherapy, immune cells, preferably T cells, are typically extracted from an individual, preferably from the patient, genetically modified and cultured in vitro and administered to the patient. Adoptive immunotherapy is advantageous in that it allows targeted growth inhibiting, preferably cytotoxic, treatment of tumor cells with less non-targeted toxicity to non-tumor cells that occurs with conventional treatments.

In a preferred embodiment in accordance with the invention, T cells are isolated from a patient having cancer, transduced with a gene transfer vector encoding a chimeric antigen receptor capable of binding to an antigen expressed by said cancer, and administered to the patient to treat said cancer. In a preferred embodiment, the T cells are CD8⁺ T cells or CD4⁺ T cells.

The terms “intermittent administration” or “administered intermittently” in connection with a tyrosine kinase inhibitor as used herein refer to the use of said tyrosine kinase inhibitor in an administration regime that causes intermittent changes between a state wherein the patient has tyrosine kinase inhibitor serum levels within the therapeutic window and a state wherein the patient has tyrosine kinase inhibitor serum levels below the therapeutic window. A therapeutic window of a given tyrosine kinase inhibitor can be determined by any methods known in the art. Alternatively, the terms “intermittent administration” or “administered intermittently” in connection with a tyrosine kinase inhibitor as used herein refer to the use of said tyrosine kinase inhibitor in an administration regime that causes intermittent changes between a state wherein the patient has tyrosine kinase inhibitor serum levels which cause complete inhibition of the tyrosine kinase and a state wherein the patient has tyrosine kinase inhibitor serum levels which cause partial inhibition of the tyrosine kinase, or intermittent changes between a state wherein the patient has tyrosine kinase inhibitor serum levels which cause complete inhibition of the tyrosine kinase and a state wherein the patient has tyrosine kinase inhibitor serum levels which cause no inhibition of the tyrosine kinase, or intermittent changes between a state wherein the patient has tyrosine kinase inhibitor serum levels which cause partial inhibition of the tyrosine kinase and a state wherein the patient has tyrosine kinase inhibitor serum levels which cause no inhibition of the tyrosine kinase. Such inhibition can be measured by any methods known in the art, e.g. by measuring the activity of the tyrosine kinase itself using appropriate enzyme assays, or by measuring cellular functions downstream of said kinase. According to the invention, a partial inhibition refers to an inhibition of at least 25% to 75% at the most, compared to a situation in the absence of the inhibitor. As used herein, “no inhibition” refers to an inhibition of less than 25%, preferably of less than 10%, compared to a situation in the absence of the inhibitor. According to the invention, in the case of T lymphocytes expressing a chimeric antigen receptor, the inhibition of less than 25%, preferably less than 10%, can preferably be an inhibition of the cytotoxic lysis, cytokine secretion, and proliferation of said T lymphocytes. According to the invention, in the case of T lymphocytes expressing a chimeric antigen receptor, the inhibition of at least 25%, but no more than 75% can preferably be an inhibition of the cytotoxic lysis, cytokine secretion, and proliferation of said T lymphocytes. According to the invention, an intermittent administration of dasatinib preferably causes intermittent changes between a state wherein the serum levels of dasatinib are above 50 nM and a state wherein the serum levels of dasatinib are at or below 50 nM. Intermittent administration may preferably be achieved by using an administration interval longer than the terminal phase half-life of the tyrosine kinase inhibitor, more preferably by using an administration interval longer than 2 times the terminal phase half-life of the tyrosine kinase inhibitor, still more preferably by using an administration interval longer than 3 times, still more preferably 4 times, still more preferably 5 times the terminal phase half-life of the tyrosine kinase inhibitor. For example, intermittent administration of dasatinib may preferably be achieved by using an administration interval of at least 6 hours for dasatinib, more preferably by using an administration interval of at least 12 hours for dasatinib. It will be understood by a person skilled in the art that for each administration regime, appropriate dosages of the respective tyrosine kinase inhibitors can be selected based on pharmacokinetic and pharmacodynamics routine experiments.

The terms “continuous administration” or “administered continuously” in connection with a tyrosine kinase inhibitor as used herein refer to the use of said tyrosine kinase inhibitor in an administration regime that causes a complete inhibition of the tyrosine kinase in a continuous manner. According to the invention, a complete inhibition refers to an inhibition of at least 75%, compared to a situation in the absence of the inhibitor. Such inhibition can be measured by any methods known in the art, e.g. by measuring the activity of the tyrosine kinase itself using appropriate enzyme assays, or by measuring cellular functions downstream of said kinase. According to the invention, in the case of T lymphocytes expressing a chimeric antigen receptor, the inhibition of at least 75% can preferably be an inhibition of the cytotoxic lysis, cytokine secretion, and proliferation of said T lymphocytes. Alternatively, the terms “continuous administration” or “administered continuously” in connection with a tyrosine kinase inhibitor as used herein refer to the use of said tyrosine kinase inhibitor in an administration regime that results in tyrosine kinase inhibitor serum levels which are continuously within the therapeutic window. According to the invention, a continuous administration of dasatinib encompasses any administration wherein the serum levels of dasatinib are constantly maintained at or above 50 nM. In an exemplary preferred embodiment, dasatinib is to be administered continuously, wherein said continuous administration comprises oral administration of 50 mg-200 mg dasatinib every 6-8 hours, preferably 140 mg every 6 hours.

The term “cell mediated effector functions” or “cell effector functions” as referred to herein describes the effects that a cell, preferably an immune cell, exerts on another cell. An exemplary “cell mediated effector function” according to the invention is cytotoxic lysis, wherein a cell, preferably an immune cell, exerts cytolytic activity directed towards another cell, preferably a tumor or cancer cell.

The terms “on-target/off tumor toxicity” or “on-target/off tumor recognition” refer to a toxicity or recognition, respectively, which is caused by an on-target effect on non-tumor cells. Such a toxicity may be a toxicity due to target antigen-specific attack of an immunotherapy, typically by immune cells of said immunotherapy, on non-malignant host tissues, respectively cells, which express the targeted antigen.

The term “off target toxicity” as used herein refers to the toxicity due to non-specific attack, e.g. a non-specific attack of an immunotherapy, preferably by immune cells of said immunotherapy, on non-malignant host tissues, i.e. tissues or cells which do not express the target antigen against which the immunotherapy is targeted.

The term “macrophage activation syndrome” or “MAS” as used herein refers to the excessive activation and proliferation macrophages caused by the release of cellular debris through lysis of tumor cells.

An “inhibition of cytokine secretion” as referred to herein can be determined by any methods known in the art. Such an inhibition is preferably a reduction of cytokine serum levels, more preferably a reduction of cytokine serum levels by at least 50%.

“Cytokine release syndrome” as used herein refers to the term as it is known in the art.

According to the invention, cytokine release syndrome refers to the release of cytokines by immune cells, e.g. T lymphocytes, which can for example express a chimeric antigen receptor, in immunotherapy against cancer, such that this release of cytokines causes unwanted side effects in the patient. Exemplary cytokines which are released by T lymphocytes in adoptive immunotherapy against cancer and may cause the occurrence of cytokine release syndrome are GM-CSF, IFN-γ, IL-2, IL-4, IL-5, IL-6, IL-8, and IL-10, preferably IFN-γ and IL2.

The term “rejection” or “rejection of immunotherapy cells” is known in the art. It preferably refers to an immune reaction occurring in a cancer patient that is treated with adoptive immunotherapy against said cancer, wherein said adoptive immunotherapy comprises transplantation of allogeneic or syngeneic T lymphocytes expressing a chimeric antigen receptor capable of binding to a cell surface antigen which is expressed in a fraction of cells of said cancer, wherein the immune reaction causes depletion of said allogeneic or syngeneic T lymphocytes.

The term “inadvertent activation” or “inadvertent activation of immunotherapy cells” as used herein is known in the art. It preferably refers to adoptive immunotherapy against cancer with T lymphocytes expressing a chimeric antigen receptor capable of binding to a cell surface antigen, wherein other cells, preferably immune cells, bind to said T lymphocytes independent of the specific binding of said chimeric antigen receptor to the target antigen, causing an activation of said T lymphocytes in the absence of specific antigen binding of said T lymphocytes via their chimeric antigen receptor.

The term “tonic signaling” or “tonic signaling and activation of immunotherapy cells” is known in the art. It preferably refers to the activation of T lymphocytes expressing a chimeric antigen receptor in adoptive immunotherapy against cancer independent of cellular interaction.

“Tumor lysis syndrome” as used herein refers to the term as it is known in the art. According to the invention, tumor lysis syndrome can occur when a large amount of tumor cells are lysed during immunotherapy such as adoptive immunotherapy against cancer, e.g. with T lymphocytes expressing a chimeric antigen receptor, and cellular debris of the lysed tumor cells is released in the bloodstream, causing side effects associated with said immunotherapy. The release of said tumor cell debris due to cytotoxic lysis by T lymphocytes can cause, for example, kidney damage.

“Neurotoxicity” as used herein refers to any processes that cause toxic effects to cells associated with the central and/or peripheral nervous system.

“Viability” as used herein refers to the fraction of live cells as compared to dead cells. Assays to determine the fraction of live cells are known in the art. An exemplary non-limiting method demonstrated herein comprises staining with Annexin V and 7-AAD to determine the fraction of viable cells.

The term antibody as used herein refers to any functional antibody that is capable of specific binding to the antigen of interest. Without particular limitation, the term antibody encompasses antibodies from any appropriate source species, including avian such as chicken and mammalian such as mouse, goat, non-human primate and human. Preferably, the antibody is a humanized or human antibody. Humanized antibodies are antibodies which contain human sequences and a minor portion of non-human sequences which confer binding specificity to an antigen of interest (e.g. human FLT3). The antibody is preferably a monoclonal antibody which can be prepared by methods well-known in the art. The term antibody encompasses an IgG-1, -2, -3, or -4, IgE, IgA, IgM, or IgD isotype antibody. The term antibody encompasses monomeric antibodies (such as IgD, IgE, IgG) or oligomeric antibodies (such as IgA or IgM). The term antibody also encompasses—without particular limitations—isolated antibodies and modified antibodies such as genetically engineered antibodies, e.g. chimeric antibodies or bispecific antibodies.

An antibody fragment or fragment of an antibody as used herein refers to a portion of an antibody that retains the capability of the antibody to specifically bind to the antigen. This capability can, for instance, be determined by determining the capability of the antigen-binding portion to compete with the antibody for specific binding to the antigen by methods known in the art. Without particular limitation, the antibody fragment can be produced by any suitable method known in the art, including recombinant DNA methods and preparation by chemical or enzymatic fragmentation of antibodies. Antibody fragments may be Fab fragments, F(ab′) fragments, F(ab′)2 fragments, single chain antibodies (scFv), single-domain antibodies, diabodies or any other portion(s) of the antibody that retain the capability of the antibody to specifically bind to the antigen.

An “antibody” (e.g. a monoclonal antibody) or “a fragment thereof” as described herein may have been derivatized or be linked to a different molecule. For example, molecules that may be linked to the antibody are other proteins (e.g. other antibodies), a molecular label (e.g. a fluorescent, luminescent, colored or radioactive molecule), a pharmaceutical and/or a toxic agent. The antibody or antigen-binding portion may be linked directly (e.g. in form of a fusion between two proteins), or via a linker molecule (e.g. any suitable type of chemical linker known in the art).

Terms such as “treatment of cancer” or “treating cancer” according to the present invention refer to a therapeutic treatment. An assessment of whether or not a therapeutic treatment works can, for instance, be made by assessing whether the treatment inhibits cancer growth in the treated patient or patients. Preferably, the inhibition is statistically significant as assessed by appropriate statistical tests which are known in the art. Inhibition of cancer growth may be assessed by comparing cancer growth in a group of patients treated in accordance with the present invention to a control group of untreated patients, or by comparing a group of patients that receive a standard cancer treatment of the art plus a treatment according to the invention with a control group of patients that only receive a standard cancer treatment of the art. Such studies for assessing the inhibition of cancer growth are designed in accordance with accepted standards for clinical studies, e.g. double-blinded, randomized studies with sufficient statistical power. The term “treating cancer” includes an inhibition of cancer growth where the cancer growth is inhibited partially (i.e. where the cancer growth in the patient is delayed compared to the control group of patients), an inhibition where the cancer growth is inhibited completely (i.e. where the cancer growth in the patient is stopped), and an inhibition where cancer growth is reversed (i.e. the cancer shrinks). An assessment of whether or not a therapeutic treatment works can be made based on known clinical indicators of cancer progression.

A treatment of cancer according to the present invention does not exclude that additional or secondary therapeutic benefits also occur in patients. For example, an additional or secondary benefit may be an enhancement of engraftment of transplanted hematopoietic stem cells that is carried out prior to, concurrently to, or after the treatment of cancer.

The term “composition for use in a method for the treatment of cancer . . . wherein the method is a method for treating cancer comprising immunotherapy” can pertain to a situation where the composition has a direct effect on the cancer, or it can pertain to a situation where the composition has an indirect effect on the cancer, e.g. by enhancing the immunotherapy. For example, a composition comprising a tyrosine kinase inhibitor such as dasatinib can enhance adoptive immunotherapy, e.g. adoptive immunotherapy with CAR T-cells.

The treatment of cancer according to the invention can be a first-line therapy, a second-line therapy, a third-line therapy, or a fourth-line therapy. The treatment can also be a therapy that is beyond is beyond fourth-line therapy. The meaning of these terms is known in the art and in accordance with the terminology that is commonly used by the US National Cancer Institute.

The term “capable of binding” as used herein refers to the capability to form a complex with a molecule that is to be bound (e.g. CD19, FLT3, BCMA, or ROR1). Binding typically occurs non-covalently by intermolecular forces, such as ionic bonds, hydrogen bonds and Van der Waals forces and is typically reversible. Various methods and assays to determine binding capability are known in the art. Binding is usually a binding with high affinity, wherein the affinity as measured in K_{D} values is preferably is less than 1 μM, more preferably less than 100 nM, even more preferably less than 10 nM, even more preferably less than 1 nM, even more preferably less than 100 μM, even more preferably less than 10 μM, even more preferably less than 1 μM.

As used herein, each occurrence of terms such as “comprising” or “comprises” may optionally be substituted with “consisting of” or “consists of”.

A pharmaceutically acceptable carrier, including any suitable diluent or, can be used herein as known in the art. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. Pharmaceutically acceptable carriers include, but are not limited to, saline, buffered saline, dextrose, water, glycerol, sterile isotonic aqueous buffer, and combinations thereof. It will be understood that the formulation will be appropriately adapted to suit the mode of administration.

Compositions and formulations in accordance with the present invention are prepared in accordance with known standards for the preparation of pharmaceutical compositions and formulations. For instance, the compositions and formulations are prepared in a way that they can be stored and administered appropriately, e.g. by using pharmaceutically acceptable components such as carriers, excipients or stabilizers. Such pharmaceutically acceptable components are not toxic in the amounts used when administering the pharmaceutical composition or formulation to a patient. The pharmaceutical acceptable components added to the pharmaceutical compositions or formulations may depend on the chemical nature of the tyrosine kinase inhibitor present in the composition or formulation (depend on whether the targeting agent is e.g. an antibody or fragment thereof or a cell expressing a chimeric antigen receptor), the particular intended use of the pharmaceutical compositions and the route of administration.

In a preferred embodiment in accordance with the invention, the composition or formulation is suitable for administration to humans, preferably the formulation is sterile and/or non-pyrogenic.

A “combination” of an immune cell and a tyrosine kinase inhibitor for the uses according to the invention is not limited to a particular mode of administration. The immune cell and a tyrosine kinase inhibitor can, for example, be administered separately but at the same time, or in one composition and at the same time, or they can be administered separately and at separate time points.

A preferred embodiment is the use of a Src kinase inhibitor in combination with adoptive immunotherapy to treat, mitigate or prevent side effects associated said adoptive immunotherapy. A more preferred embodiment is the use of a Src kinase inhibitor, preferably dasatinib, saracatinib, bosutinib, nilotinib, or PP1-inhibitor, in combination with adoptive immunotherapy against cancer, to treat, mitigate or prevent side effects associated with said adoptive immunotherapy against cancer, wherein said adoptive immunotherapy against cancer comprises transplantation of immune cells, preferably T lymphocytes, which express a chimeric antigen receptor that recognizes an antigen expressed by a fraction of cells of said cancer. An even more preferred embodiment is the use of dasatinib to treat, mitigate or prevent side effects associated with adoptive immunotherapy against cancer with T lymphocytes genetically modified to express a chimeric antigen receptor, wherein the chimeric antigen receptor is capable of binding to a cell surface antigen expressed in a fraction of cells of said cancer.

A preferred embodiment is the use of a Src kinase inhibitor, preferably dasatinib, saracatinib, bosutinib, nilotinib, or PP1-inhibitor, most preferably dasatinib, to treat, mitigate or prevent side effects associated with adoptive immunotherapy against cancer, wherein said immunotherapy comprises transplantation of T lymphocytes genetically modified to express a chimeric antigen receptor which is capable of binding to a cell surface antigen expressed on a fraction of cells of said cancer. In this embodiment, the chimeric antigen receptor expressed in the transplanted T lymphocyte binds to a cell surface antigen of the cancer cells, which causes cytotoxic lysis of said cancer cells, and side effects associated with said adoptive immunotherapy are caused primarily or in part by the release of cellular debris of said cancer cells upon the cytotoxic lysis mediated by said T lymphocyte expressing a chimeric antigen receptor. In a more preferred embodiment, the side effects associated with said adoptive immunotherapy caused by said release of cellular debris can be classified as tumor lysis syndrome or macrophage activation syndrome.

A preferred embodiment is the use of a Src kinase inhibitor, preferably dasatinib, saracatinib, bosutinib, nilotinib, or PP1-inhibitor, most preferably dasatinib, to treat, mitigate or prevent side effects associated with adoptive immunotherapy against cancer, wherein said immunotherapy comprises transplantation of T lymphocytes genetically modified to express a chimeric antigen receptor which is capable of binding to a cell surface antigen expressed on a fraction of cells of said cancer. In this embodiment, the chimeric antigen receptor expressed in the transplanted T lymphocyte binds to a cell surface antigen of the cancer cells, which causes cytotoxic lysis of said cancer cells and activation of said T lymphocytes, and side effects associated with said adoptive immunotherapy are caused primarily or in part by the release of cytokines by said T lymphocytes expressing a chimeric antigen receptor upon binding of said chimeric antigen receptor to said cell surface antigen, preferably wherein said cell surface antigen is on the surface of a cancer cell. In a more preferred embodiment, the side effects associated with said immunotherapy caused by said release of cytokines by said T lymphocytes expressing a chimeric antigen receptor can be classified as cytokine release syndrome.

In a preferred embodiment, the use of said Src kinase inhibitor, preferably dasatinib, saracatinib, bosutinib, nilotinib, or PP1-inhibitor, most preferably dasatinib, to prevent side effects associated with adoptive immunotherapy against cancer comprises administration of said Src kinase inhibitor prior to adoptive immunotherapy. In another preferred embodiment, the use of said Src kinase inhibitor, preferably dasatinib, saracatinib, bosutinib, nilotinib, or PP1-inhibitor, most preferably dasatinib, to treat or mitigate side effects associated with adoptive immunotherapy against cancer comprises administration of said Src kinase inhibitor after to adoptive immunotherapy against cancer, preferably when symptoms of side effects associated with said adoptive immunotherapy against cancer occur. Symptoms of side effects associated with adoptive immunotherapy against cancer may include elevated serum levels of IFN-γ, IL-6, or MCP1, and/or elevated body temperature.

In a preferred embodiment, the side effects associated with adoptive immunotherapy against cancer are primarily or in part due to elevated serum levels of GM-CSF, IFN-γ, IL-2, IL-4, IL-5, IL-6, IL-8, or IL-10, preferably due to elevated serum levels of IFN-γ and IL-2. In a preferred embodiment, the method of treating cancer comprises adoptive immunotherapy with allogeneic or syngeneic T lymphocytes which express a chimeric antigen receptor capable of binding to a cell surface antigen expressed by a fraction of cells of said cancer. In this embodiment, said T lymphocytes, upon binding to said cell surface antigen, release the cytokines GM-CSF, IFN-γ, IL-2, IL-4, IL-5, IL-6, IL-8, or IL-10, preferably IFN-γ and IL-2, causing elevated serum levels thereof. A preferred embodiment is the use of a Src kinase inhibitor, preferably dasatinib, saracatinib, bosutinib, nilotinib, or PP1-inhibitor, most preferably dasatinib, to reduce the release of said cytokines by inhibition of said T lymphocytes, causing a decrease in the symptoms associated with said elevated serum levels of said cytokines.

In another preferred embodiment, the side effects associated with adoptive immunotherapy against cancer are primarily or in part due to on-target/off-tumor recognition. In a preferred embodiment, the method of treating cancer comprises adoptive immunotherapy with allogeneic or syngeneic T lymphocytes which express a chimeric antigen receptor capable of binding to a cell surface antigen expressed by a fraction of cells of said cancer. In this embodiment, said T lymphocytes, bind to said cell surface antigen, which is expressed on a fraction of non-tumor, non-malignant cells, causing unwanted cytotoxic lysis of said non-tumor, non-malignant cells. A preferred embodiment is the use of a Src kinase inhibitor, preferably dasatinib, saracatinib, bosutinib, nilotinib, or PP1-inhibitor, most preferably dasatinib, to reduce the on-target/off-tumor recognition by inhibition of the cytolytic activity of said T lymphocytes, causing a decrease in the symptoms associated with said on-target/off-tumor recognition. An exemplary embodiment is the use of dasatinib in a method for treating CD19 positive cancer with T lymphocytes expressing a chimeric antigen receptor capable of binding to CD19, wherein said T lymphocytes bind to non-tumor cells expressing CD19, leading to cytotoxic lysis of said non-tumor cells, causing unwanted on-target/off-tumor side effects in the patient.

A preferred embodiment is the use of a Src kinase inhibitor, preferably dasatinib, saracatinib, bosutinib, nilotinib, or PP1-inhibitor, most preferably dasatinib, in a method for treating cancer by adoptive immunotherapy with T lymphocytes expressing a chimeric antigen receptor to inhibit said T lymphocytes' cell mediated effector functions. In a preferred embodiment, said Src kinase inhibitor causes a decrease in cytokine secretion, cytotoxic lysis, or proliferation of said T lymphocytes. In a preferred embodiment, cytokine secretion of GM-CSF, IFN-γ, IL-2, IL-4, IL-5, IL-6, IL-8, or IL-10, preferably IFN-γ and IL-2, by said T lymphocytes is reduced by at least 10%, 20%, 30%, 40% or 50% after said Src kinase inhibitor has been administered, as compared to secretion of said cytokines in the absence of said Src kinase inhibitor. In a preferred embodiment, said cytokine secretion is reduced by at least 50%.

In a preferred embodiment, the use of the Src kinase inhibitor in the method of treating cancer by adoptive immunotherapy with T lymphocytes expressing a chimeric antigen receptor capable of binding to a cell surface antigen that is expressed on a fraction of cells of said cancer does not significantly decrease the viability of said T lymphocytes. In a preferred embodiment, the viability of the T lymphocytes expressing a chimeric antigen receptor is at least 50%, 60%, 70%, 80%, or 90% after the Src kinase inhibitor has been administered. In a preferred embodiment, the viability of the T lymphocytes expressing a chimeric antigen receptor is at least 80% after the Src kinase inhibitor has been administered.

In a preferred embodiment, the use of the Src kinase inhibitor in the method of treating cancer by adoptive immunotherapy with T lymphocytes expressing a chimeric antigen receptor capable of binding to a cell surface antigen that is expressed on a fraction of cells of said cancer inhibits the proliferation of said T lymphocytes. In a preferred embodiment, the proliferation of the T lymphocytes expressing a chimeric antigen receptor is reduced by at least 10%, 20%, 30%, 40% 50%, 60%, 70%, 80%, or 90% after the Src kinase inhibitor has been administered, compared to the proliferation of said T lymphocytes in the absence of said Src kinase inhibitor.

In a preferred embodiment, the proliferation of the T lymphocytes expressing a chimeric antigen receptor is reduced by at least 50% after the Src kinase inhibitor has been administered. in a preferred embodiment, the use of the Src kinase inhibitor in the method of treating cancer by adoptive immunotherapy with T lymphocytes expressing a chimeric antigen receptor capable of binding to a cell surface antigen that is expressed on a fraction of cells of said cancer inhibits the ability of said T lymphocytes for cytotoxic lysis of target cells expressing said cell surface antigen. In a preferred embodiment, the cytotoxic lysis of the T lymphocytes expressing a chimeric antigen receptor is reduced by at least 10%, 20%, 30%, 40% 50%, 60%, 70%, 80%, or 90% after the Src kinase inhibitor has been administered, compared to the proliferation of said T lymphocytes in the absence of said Src kinase inhibitor. In a preferred embodiment, the cytotoxic lysis of the T lymphocytes expressing a chimeric antigen receptor is reduced by at least 90% after the Src kinase inhibitor has been administered.

In a preferred embodiment, the use of the Src kinase inhibitor in the method of treating cancer by adoptive immunotherapy with T lymphocytes expressing a chimeric antigen receptor capable of binding to a cell surface antigen that is expressed on a fraction of cells of said cancer inhibits the ability of said T lymphocytes for expression of PD1. In a preferred embodiment, the expression of PD1 in said T lymphocytes is statistically significantly reduced compared to the expression of PD1 in said T lymphocytes in the absence of said Src kinase inhibitor. In a preferred embodiment, the expression of PD1 in said T lymphocytes is reduced by at least 5%, 10%, 15%, 20%, or more. In a preferred embodiment, the expression of PD1 in said T lymphocytes is reduced by at least 10%.

A preferred embodiment is the use of a Src kinase inhibitor in combination with adoptive immunotherapy to improve or augment adoptive immunotherapy, wherein the Src kinase inhibitor is to be administered intermittently. A more preferred embodiment is the use of a Src kinase inhibitor, preferably dasatinib, saracatinib, bosutinib, nilotinib, or PP1-inhibitor, in combination with adoptive immunotherapy against cancer, to improve the anti-cancer effect of said adoptive immunotherapy against cancer, wherein said adoptive immunotherapy against cancer comprises transplantation of immune cells, preferably T lymphocytes, which express a chimeric antigen receptor that recognizes an antigen expressed by a fraction of cells of said cancer, and said Src kinase inhibitor is to be administered intermittently. An even more preferred embodiment is the use of dasatinib to improve the anti-cancer effect of adoptive immunotherapy against cancer with T lymphocytes genetically modified to express a chimeric antigen receptor, wherein the chimeric antigen receptor is capable of binding to a cell surface antigen expressed in a fraction of cells of said cancer, and dasatinib is to be administered intermittently. In this embodiment, dasatinib is to be administered intermittently so that there is a partial inhibition of said T lymphocytes. A partial inhibition may be an inhibition of said T lymphocyte's cell mediated effector function, wherein said inhibition is an inhibition of at least 25% to 75% at the most of one or more cell mediated effector functions of said T lymphocytes. In a preferred embodiment, dasatinib is to be administered intermittently, such that the serum levels of dasatinib are not continuously at or above 50 nM. In another preferred embodiment, dasatinib is to be administered intermittently, such that the serum levels of dasatinib are not continuously at or above 10 nM. In an exemplary embodiment, dasatinib is to be administered intermittently, wherein the intermittent administration comprises oral administration of 50-200 mg dasatinib daily, preferably 100 mg daily.

In a preferred embodiment, the tyrosine kinase inhibitor is a Src kinase inhibitor. In a more preferred embodiment, the tyrosine kinase inhibitor is dasatinib, saracatinib, bosutinib, nilotinib, or PP1-inhibitor. In a more preferred embodiment, the inhibitor is bosutinib. In a more preferred embodiment, the inhibitor is saracatinib. In a more preferred embodiment, the inhibitor is nilotinib. In a more preferred embodiment, the inhibitor is PP1-inhibitor. In an even more preferred embodiment, the inhibitor is dasatinib.

In a preferred embodiment, the method for treating cancer comprises adoptive immunotherapy with allogeneic or syngeneic T lymphocytes expressing a chimeric antigen receptor which is capable of binding to a cell surface antigen expressed on a fraction of cells of said cancer. In a more preferred embodiment, the chimeric antigen receptor is capable of binding to CD4, CD5, CD10, CD19, CD20, CD22, CD27, CD30, CD33, CD38, CD44v6, CD52, CD64, CD70, CD72, CD123, CD135, CD138, CD220, CD269, CD319, ROR1, ROR2, SLAMF7, BCMA, αvβ3-Integrin, α4β1-Integrin, LILRB4, EpCAM-1, MUC-1, MUC-16, L1-CAM, c-kit, NKG2D, NKG2D-Ligand, PD-L1, PD-L2, Lewis-Y, CAIX, CEA, c-MET, EGFR, EGFRvIII, ErbB2, Her2, FAP, FR-a, EphA2, GD2, GD3, GPC3, IL-13Ra, Mesothelin, PSMA, PSCA, VEGFR, or FLT3. In an even more preferred embodiment, the chimeric antigen receptor is capable of binding to CD19, BCMA, ROR1, FLT3, CD20, CD22, CD123, or SLAMF7.

In a preferred embodiment, the chimeric antigen receptor comprises a CD27, CD28, 4-1BB, ICOS, DAP10, NKG2D, MyD88 or OX40 costimulatory domain. In a more preferred embodiment, the chimeric antigen receptor comprises a CD28, 4-1BB, or OX40 costimulatory domain.

In a preferred embodiment, the chimeric antigen receptor comprises a CD3 zeta, CD3 epsilon, CD3 gamma, T-cell receptor alpha chain, T-cell receptor beta chain, T-cell receptor delta chain, and T-cell receptor gamma chain signaling domain.

### EXAMPLES

The present invention is exemplified by the following non-limiting examples.

### Example 1: Materials and Methods

Human Subjects

Blood samples were obtained from healthy donors who provided written informed consent to participate in research protocols approved by the Institutional Review Board of the University of Würzburg [Universitätsklinikum Würzburg, Germany (UKW)]. Peripheral blood mononuclear cells (PBMC) were isolated by centrifugation over Ficoll-Hypaque (Sigma, St. Louis, Mo.).

Cell Lines

The 293T, K562, Raji and RCH-ACV cell lines were obtained from the German Collection of Microorganisms and Cell Cultures (DSMZ, Braunschweig, Germany). K562-ROR1 were generated by lentiviral transduction with the full-length human ROR1-gene. K562-CD19 were generated by lentiviral transduction with the full-length human CD19-gene. Each of the K562, Raji and RCH-ACV cell lines were transduced with a lentiviral vector encoding a firefly luciferase (ffluc)_enhanced green fluorescent protein (GFP) transgene to enable detection by flow cytometry (GFP), bioluminescence-based cytotoxicity assays (ffLuc), and bioluminescence imaging (ffLuc) in mice. Each of the cell lines was cultured in Dulbecco's modified Eagle's medium supplemented with 10% fetal calf serum and 100 U/ml penicillin/streptomycin.

Immunophenotyping

PBMC and T-cell lines were stained with one or more of the following conjugated mAb: CD3, CD4, CD8, CD45RA, CD45RO, CD62L, PD-1 and matched isotype controls (BD Biosciences, San Jose, Calif.). CAR-transduced (i.e. EGFRt+) T-cells were detected by staining with anti-EGFR antibody (ImClone Systems Inc.) that had been biotinylated in-house (EZ-LinkSulfo-NHS-SS-Biotin, ThermoFisher Scientific, IL; according to the manufacturer's instructions) and streptavidin-PE (BD Biosciences). Staining with 7-AAD (BD Biosciences) was performed for live/dead cell discrimination as directed by the manufacturer. Flow analyses were done on a FACS Canto and data analyzed using FlowJo software (Treestar, Ashland, Oreg.).

Vector Construction

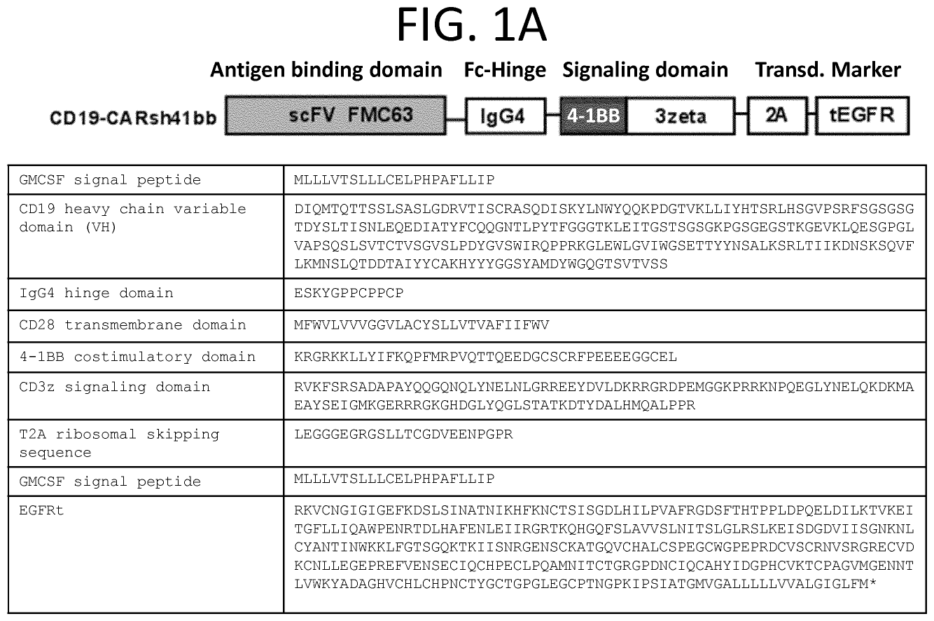

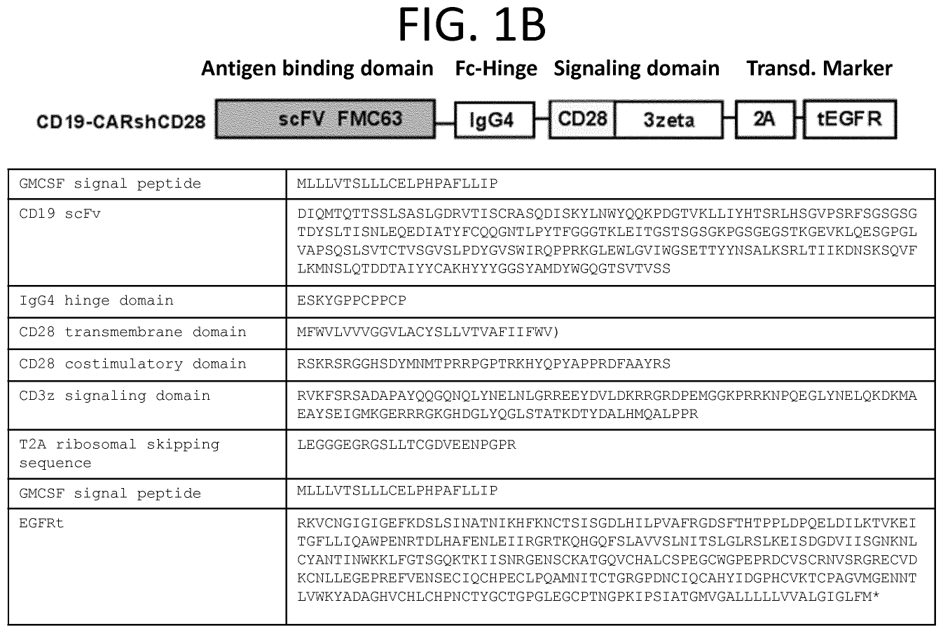

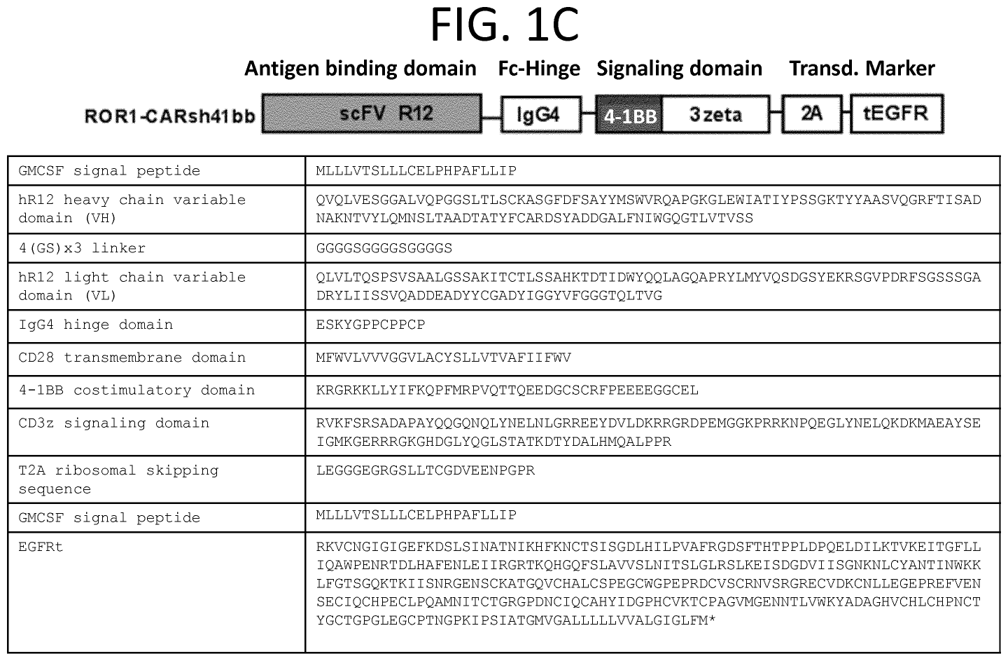

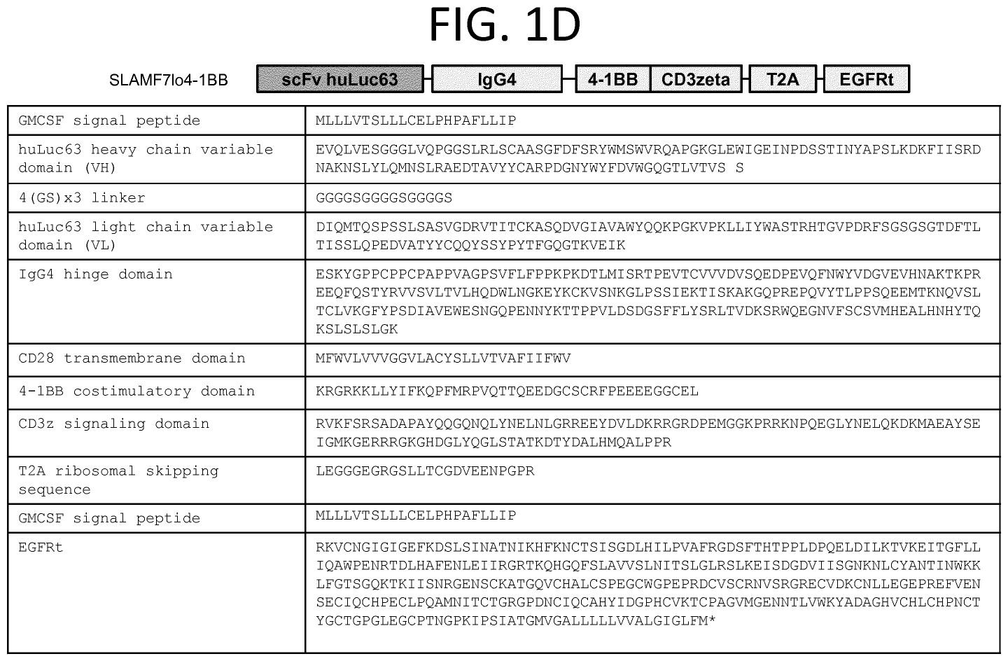

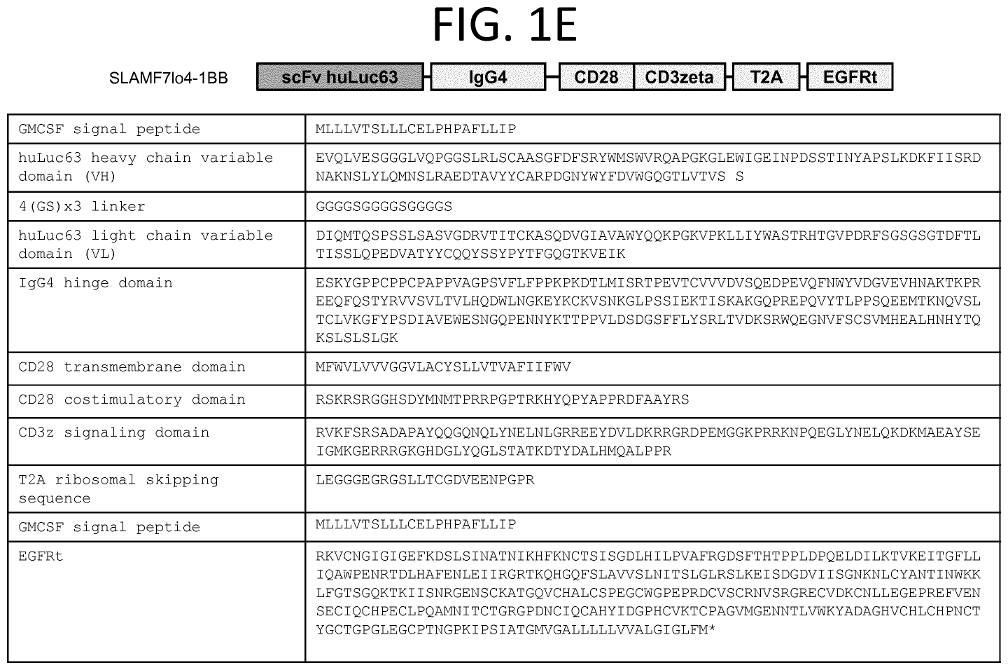

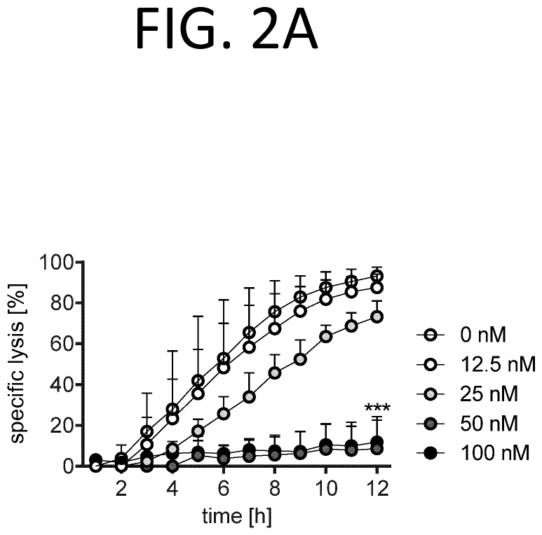

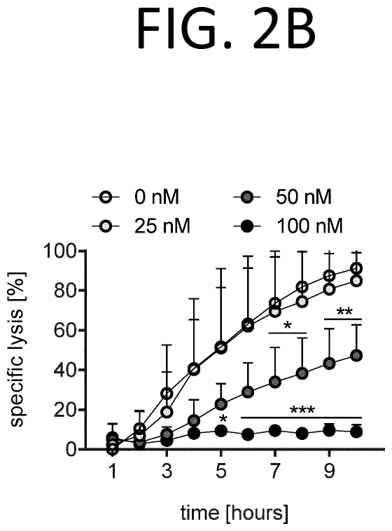

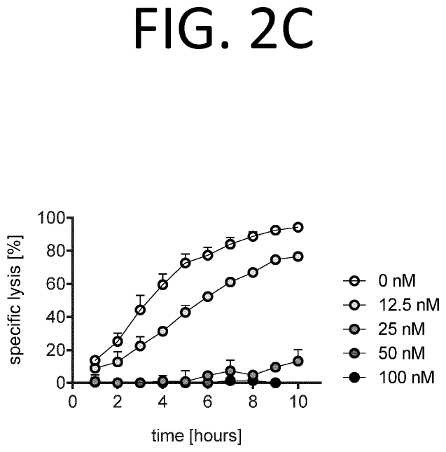

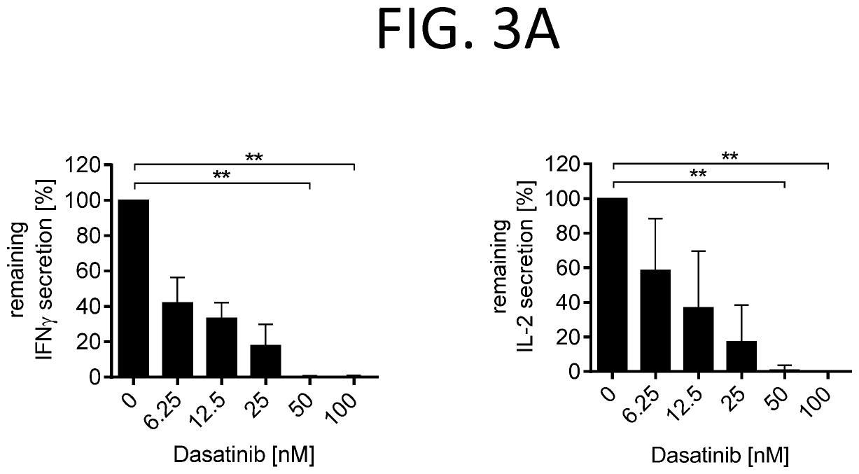

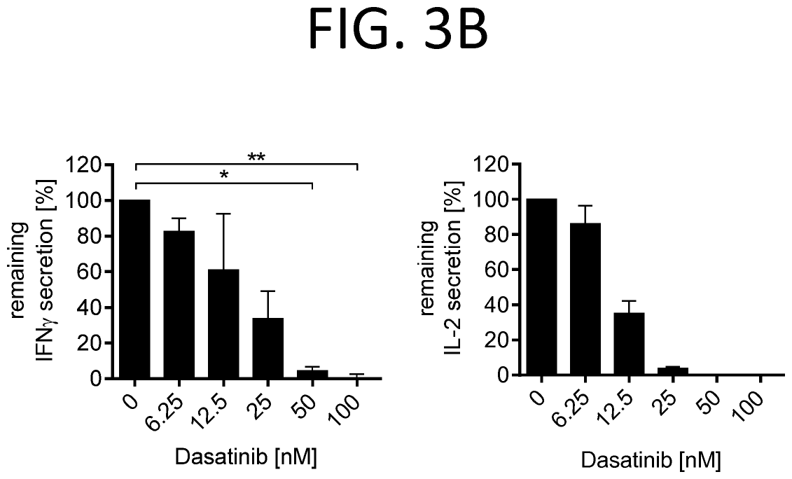

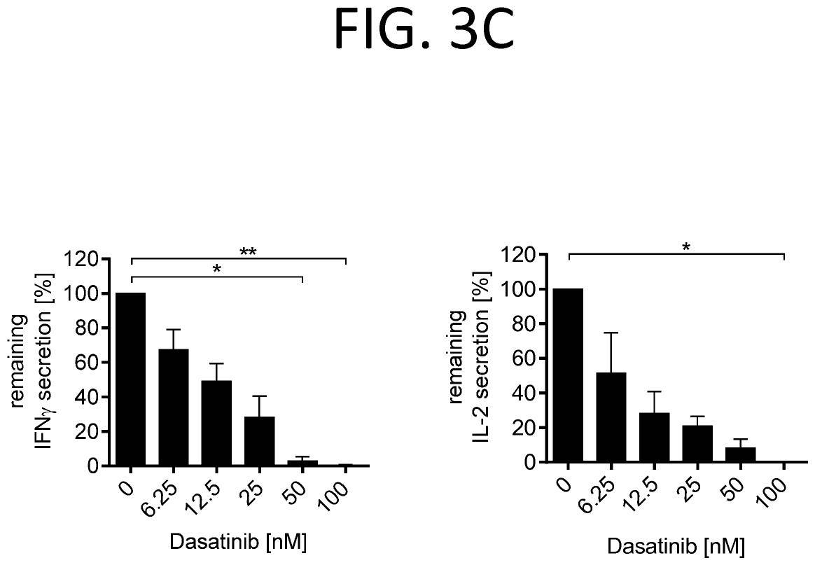

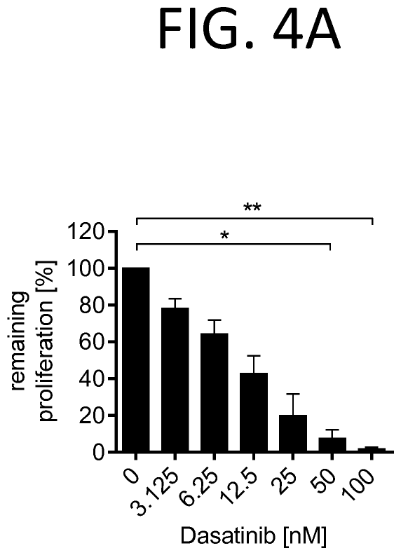

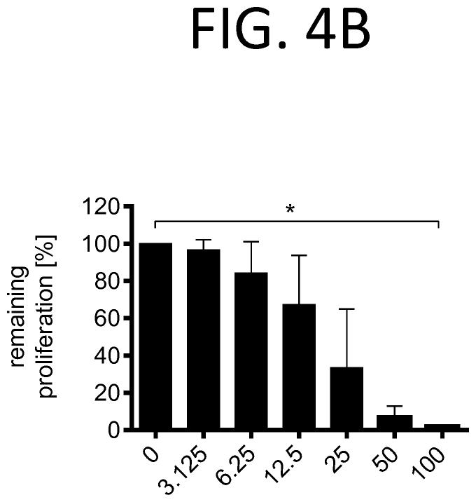

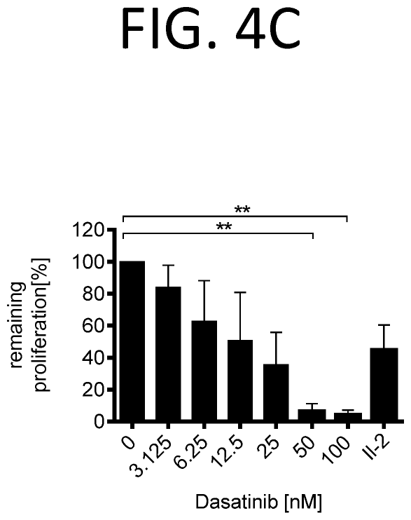

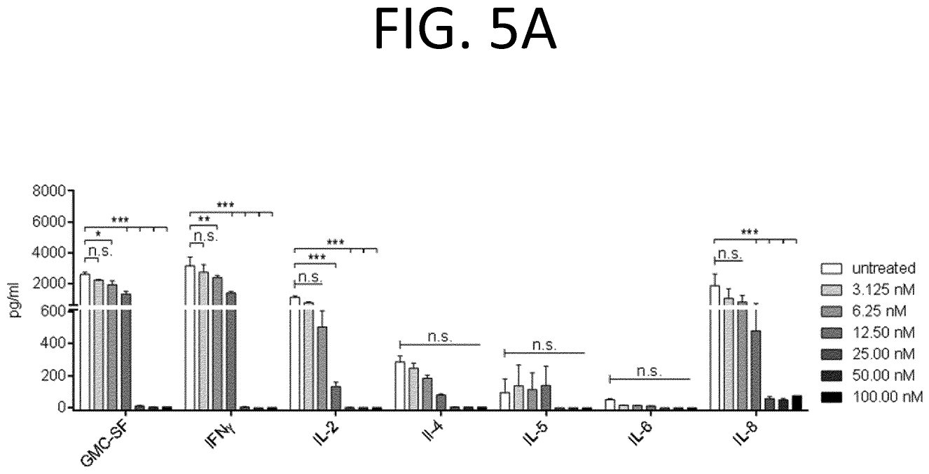

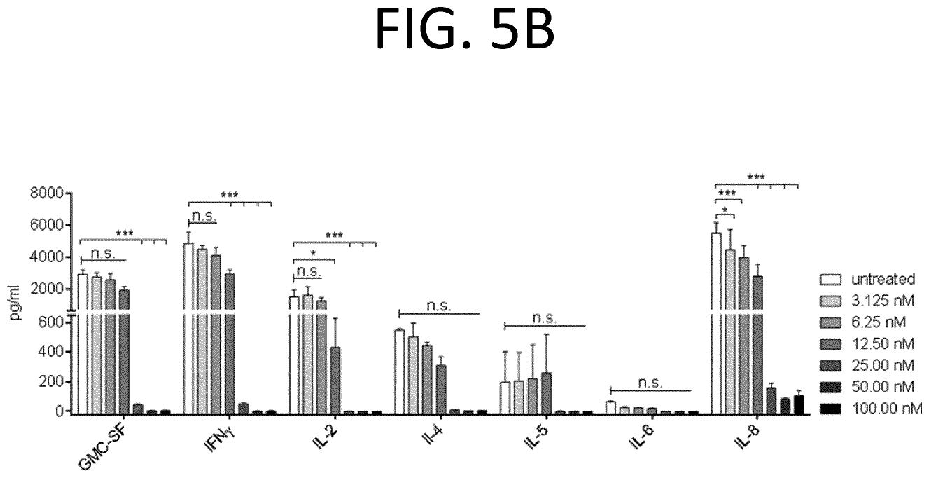

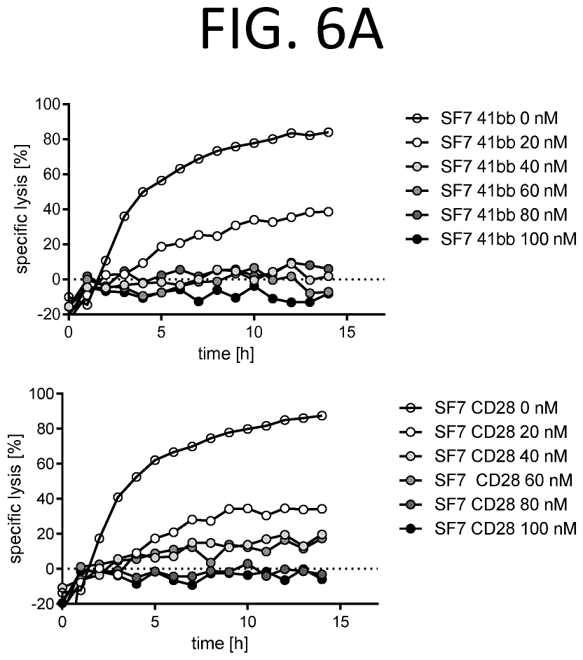

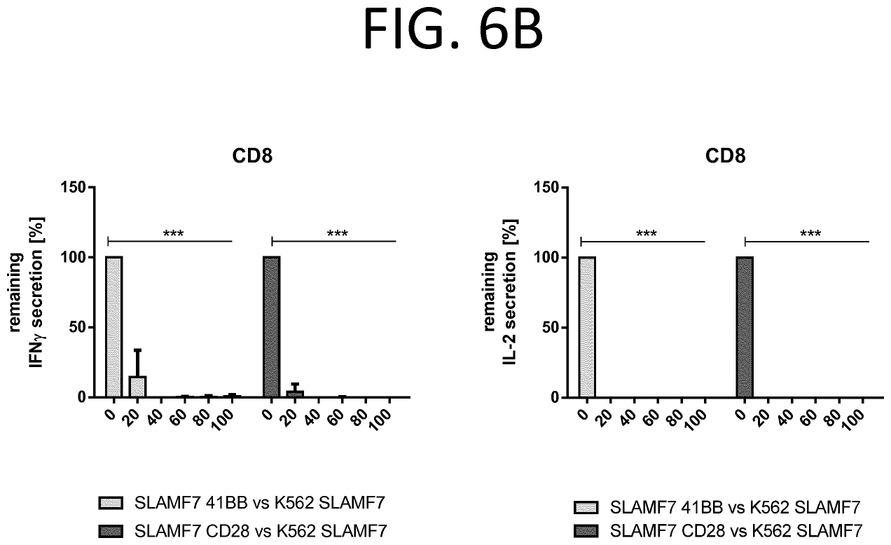

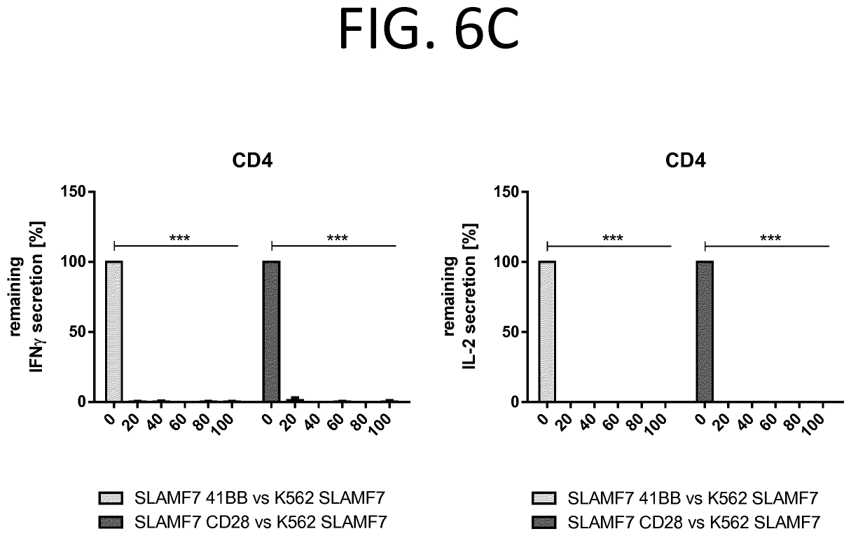

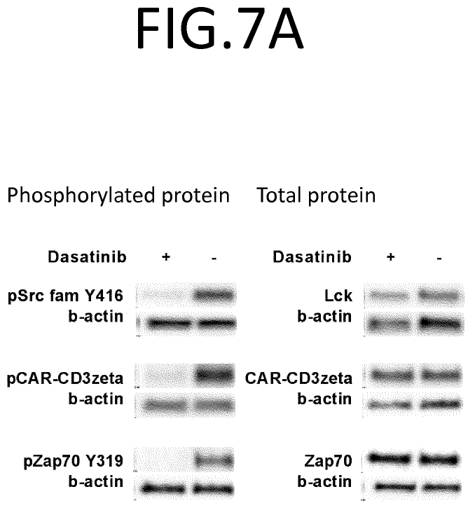

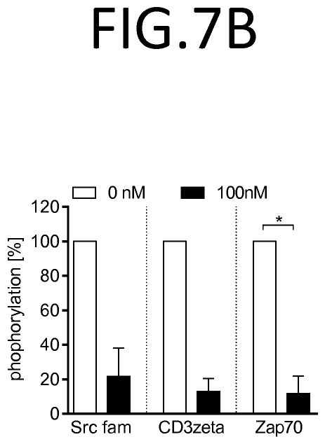

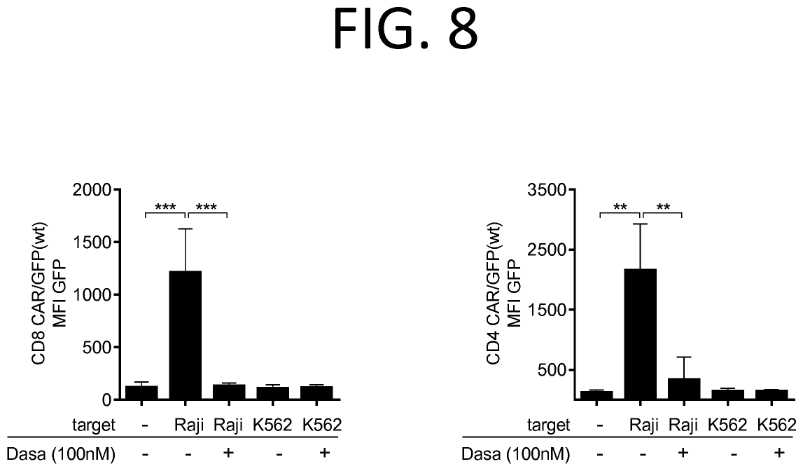

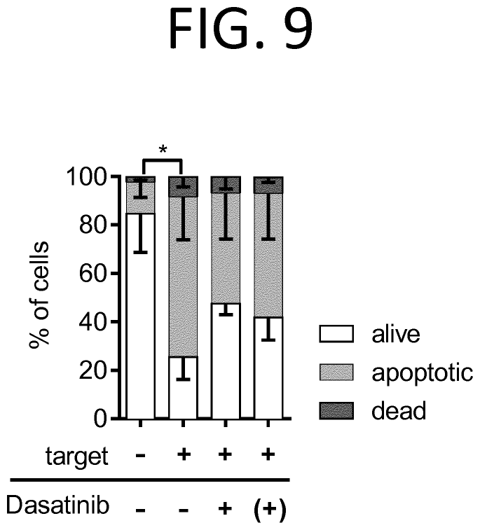

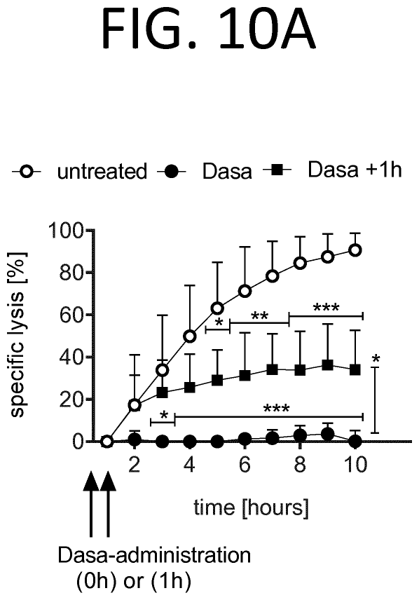

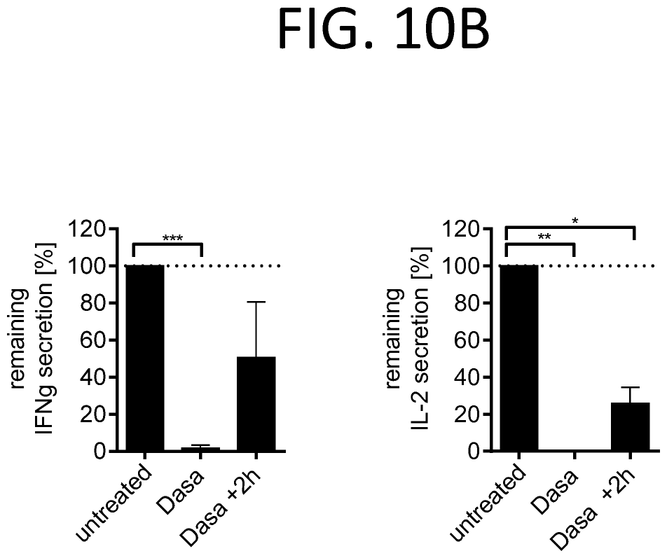

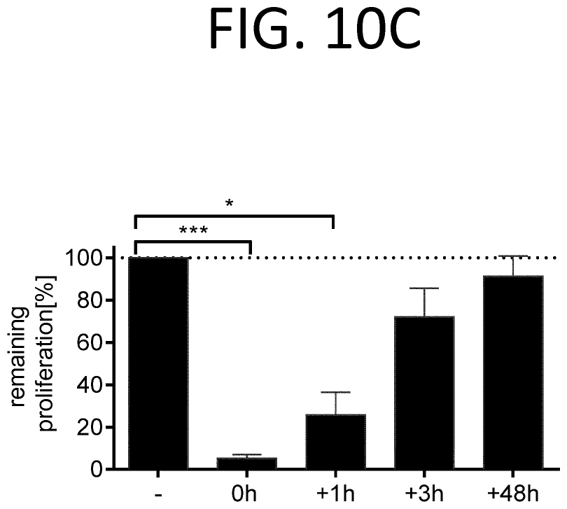

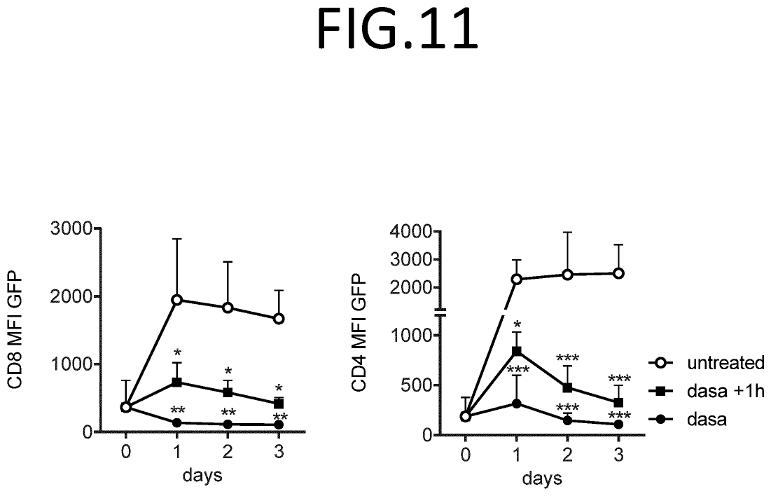

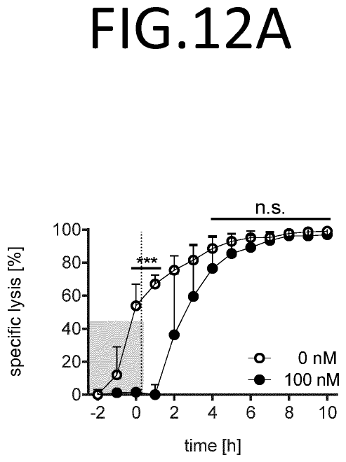

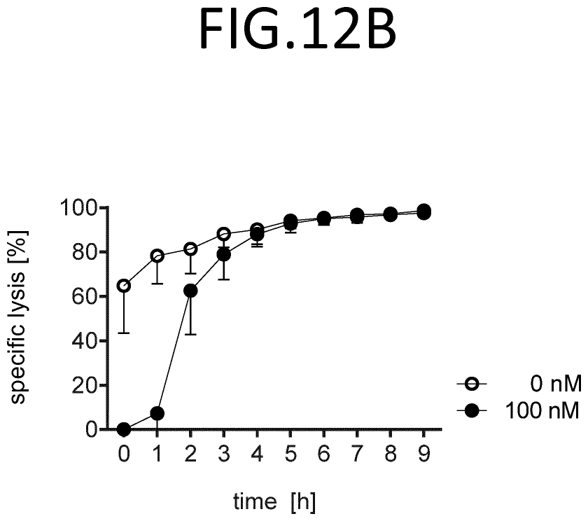

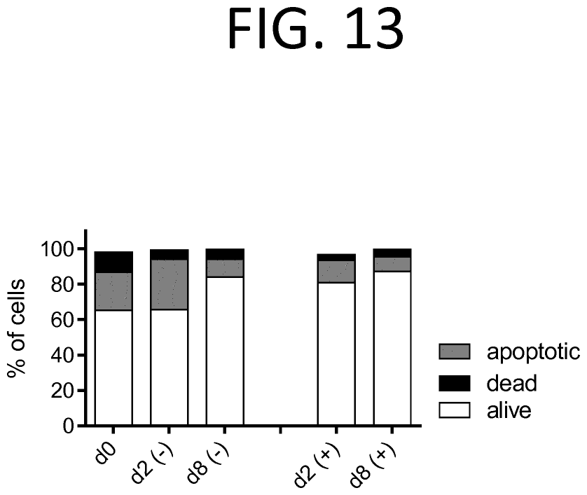

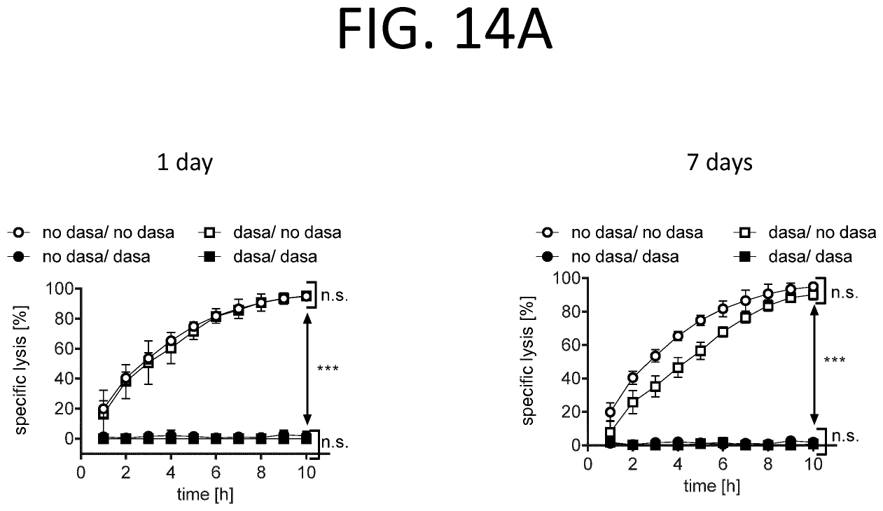

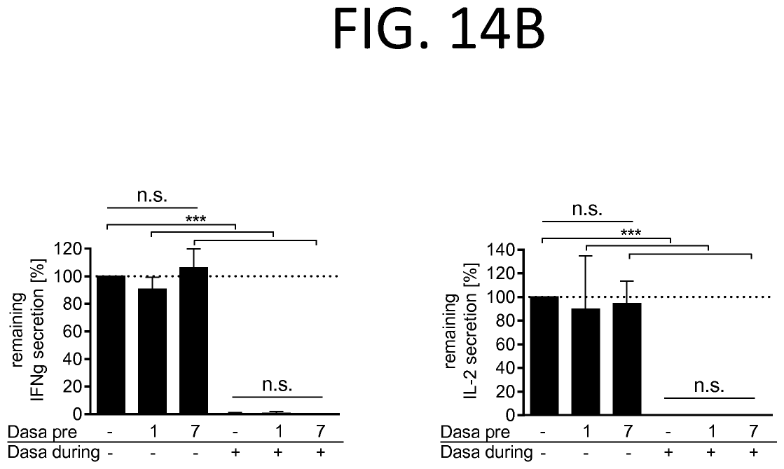

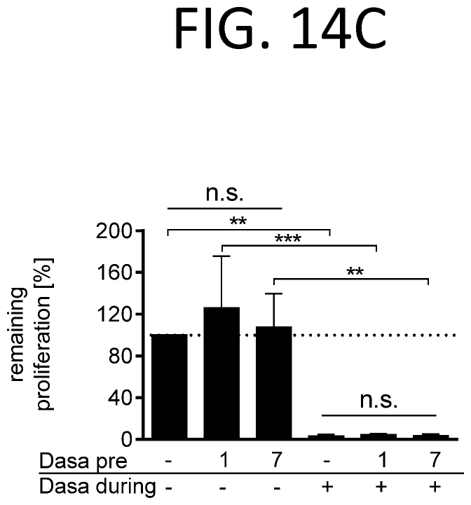

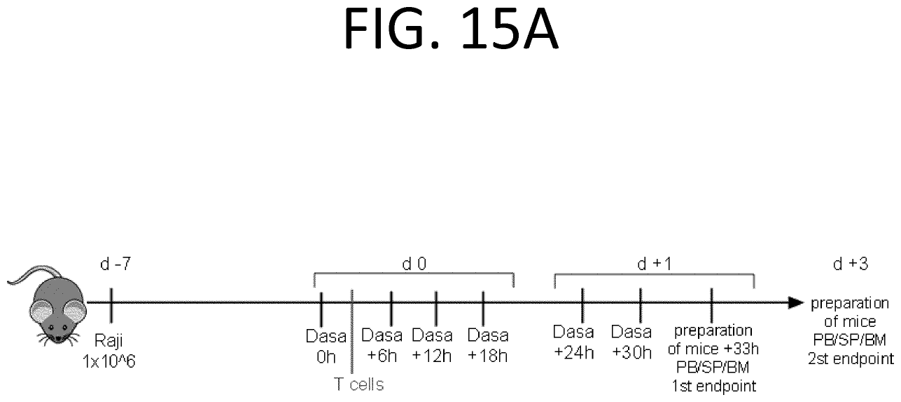

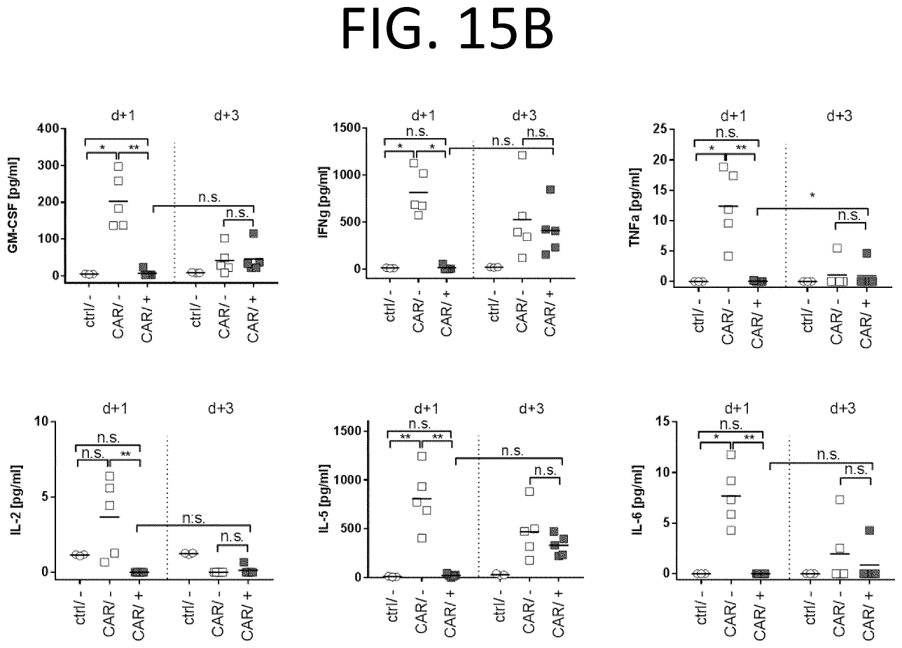

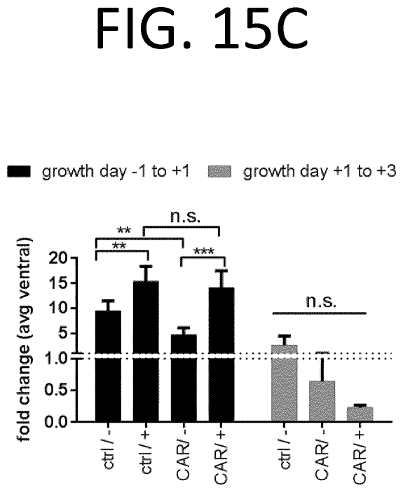

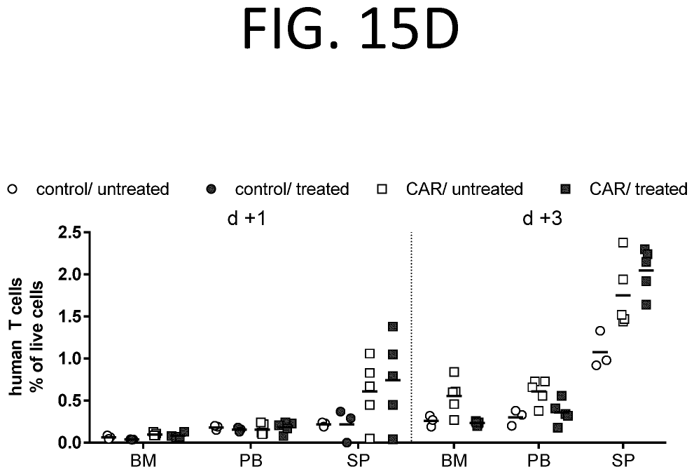

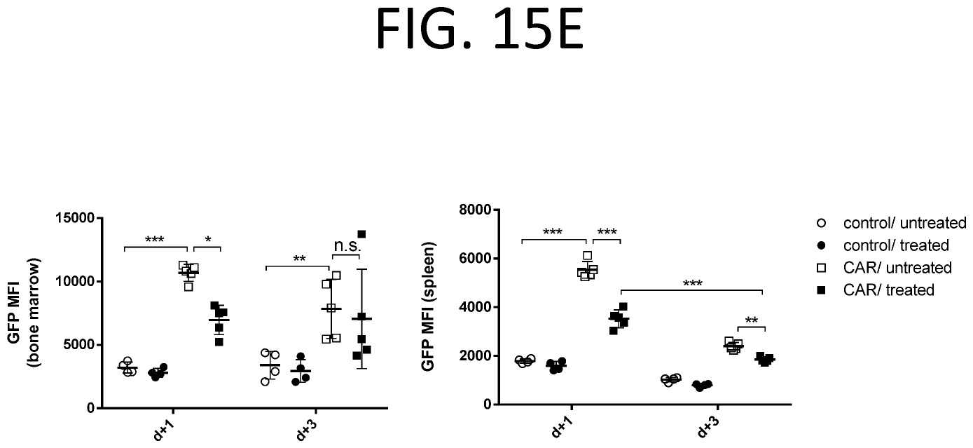

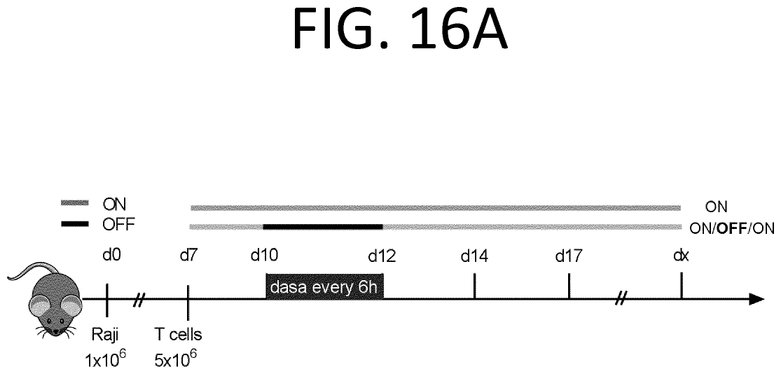

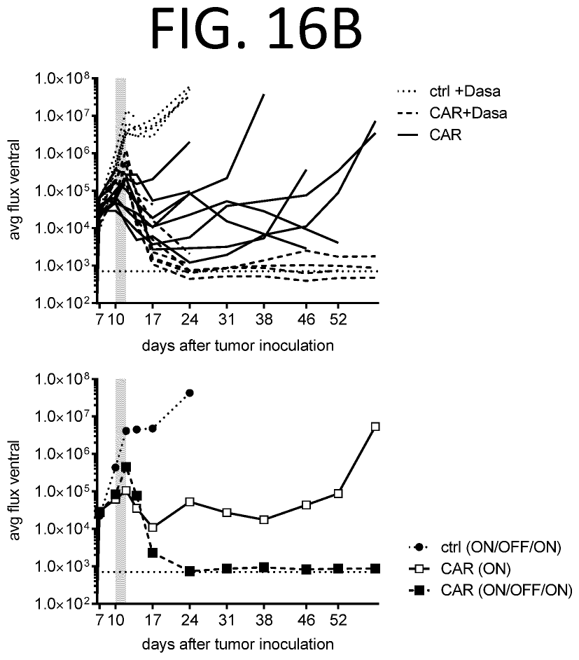

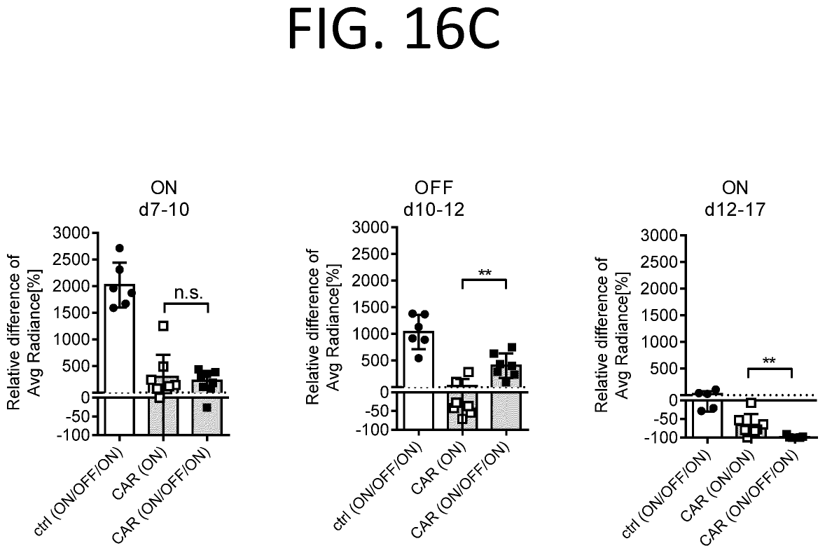

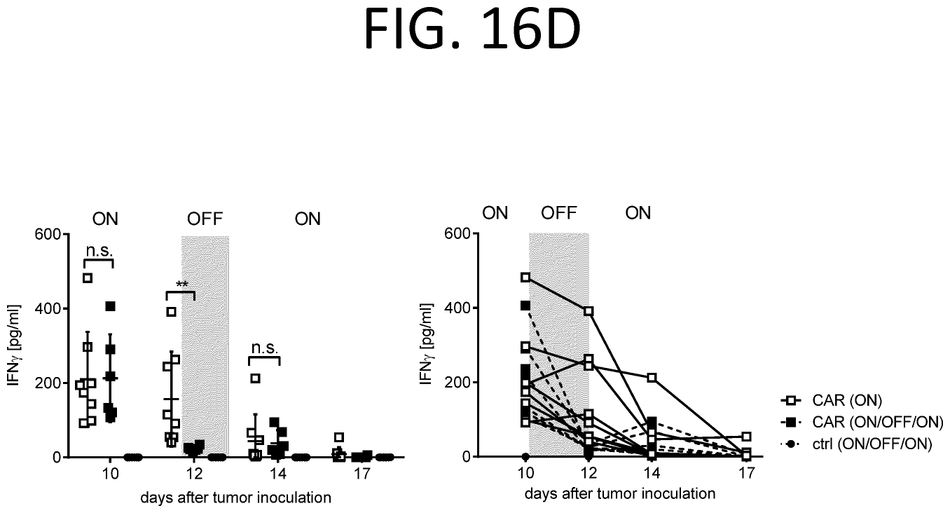

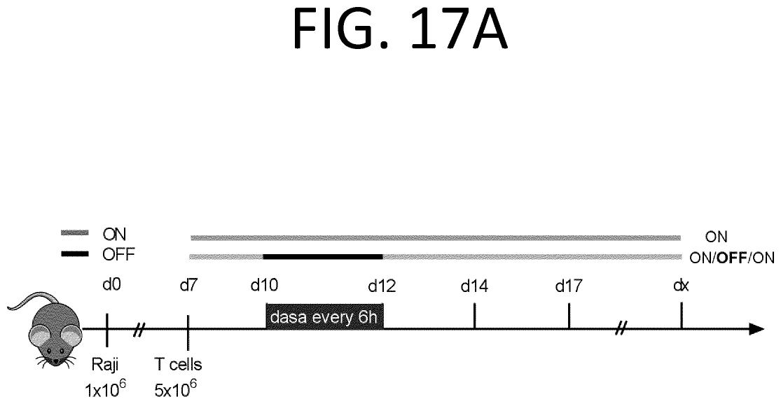

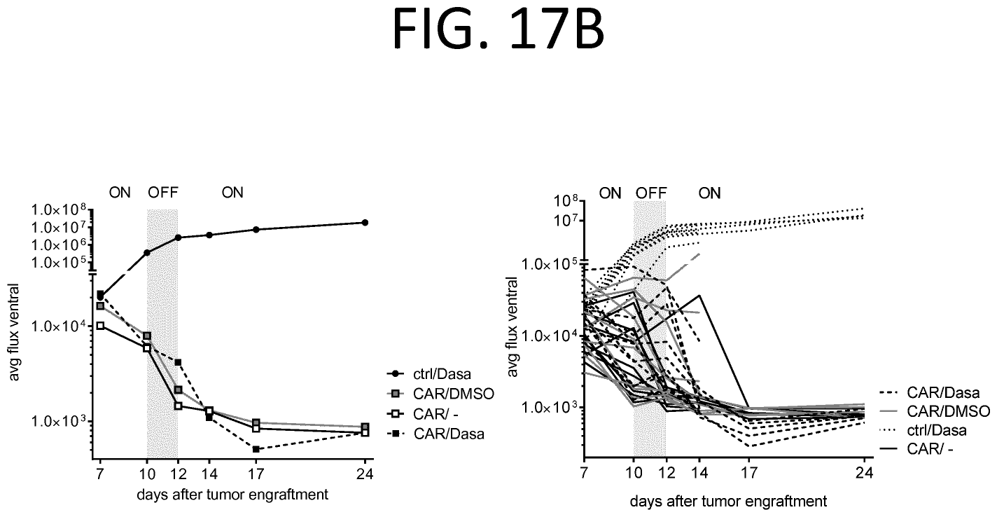

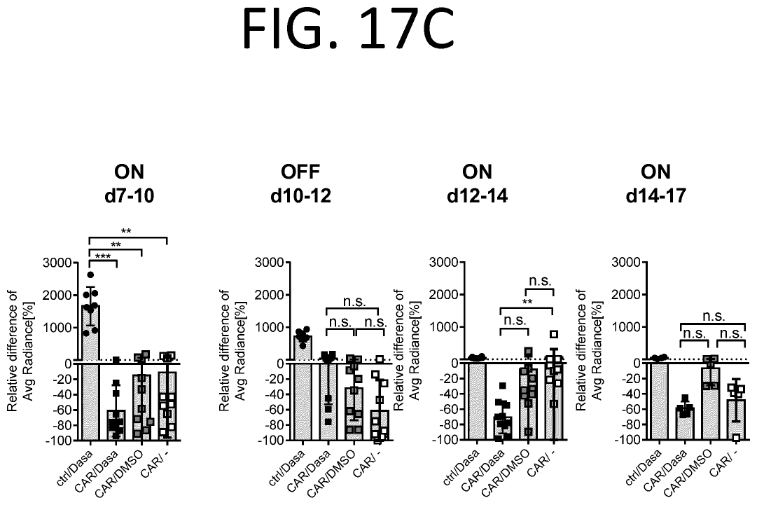

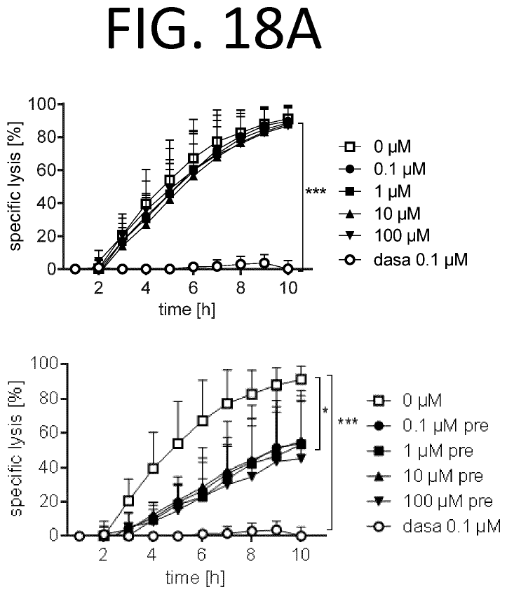

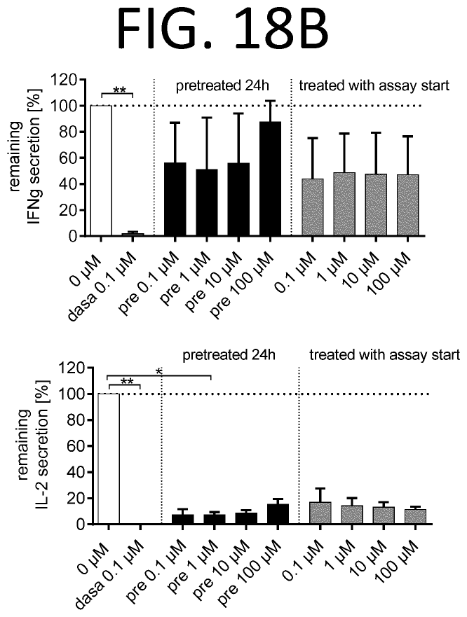

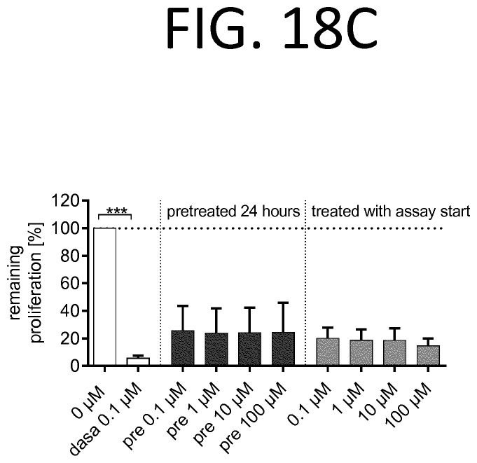

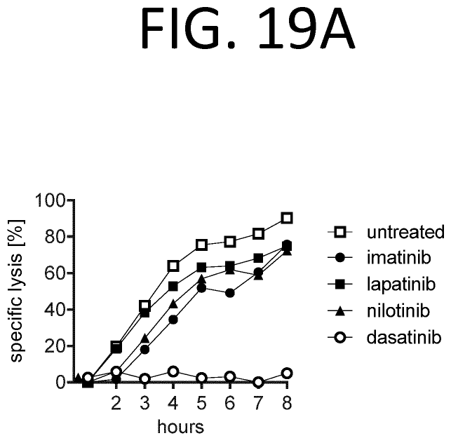

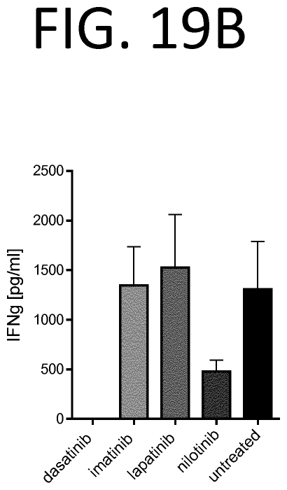

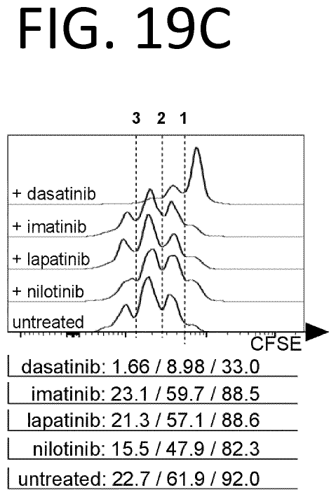

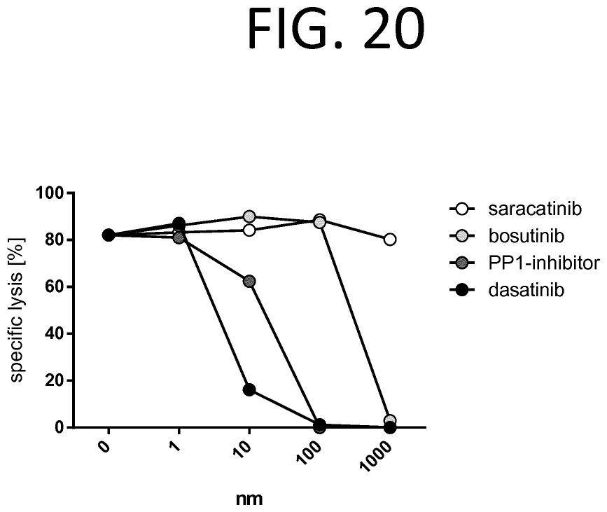

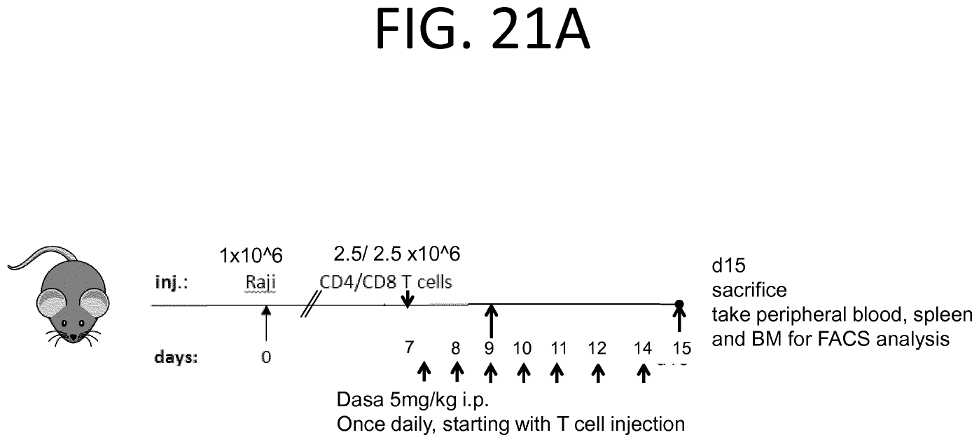

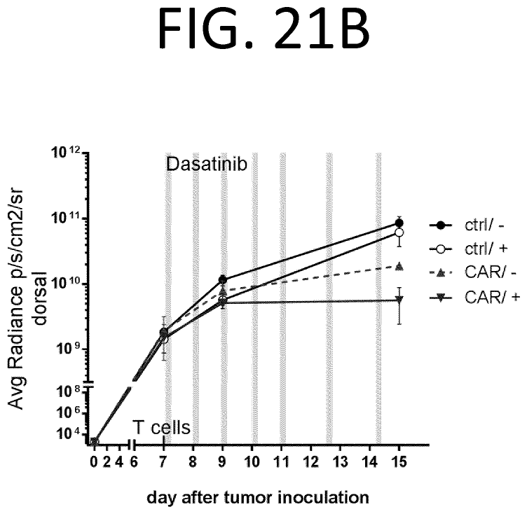

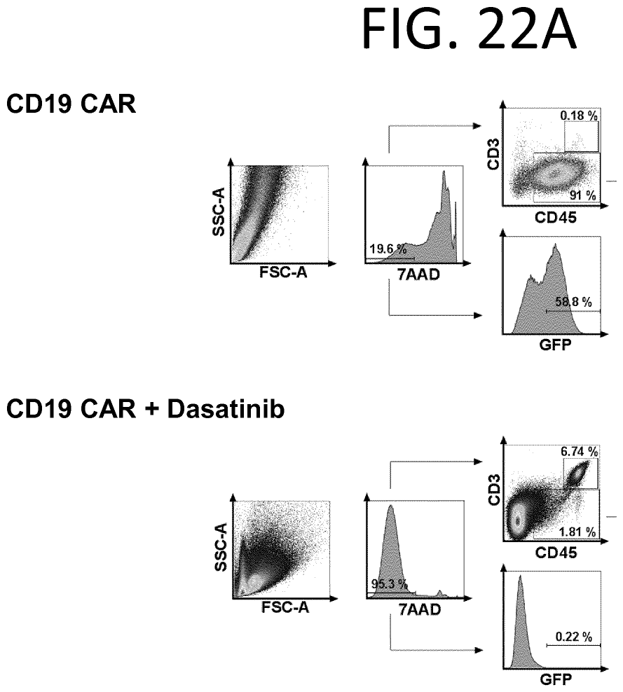

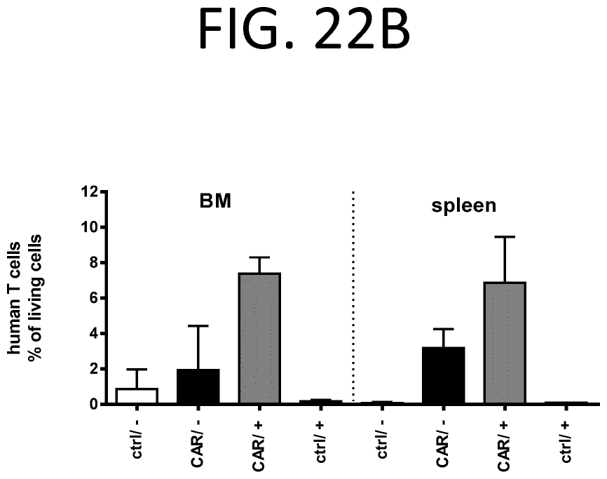

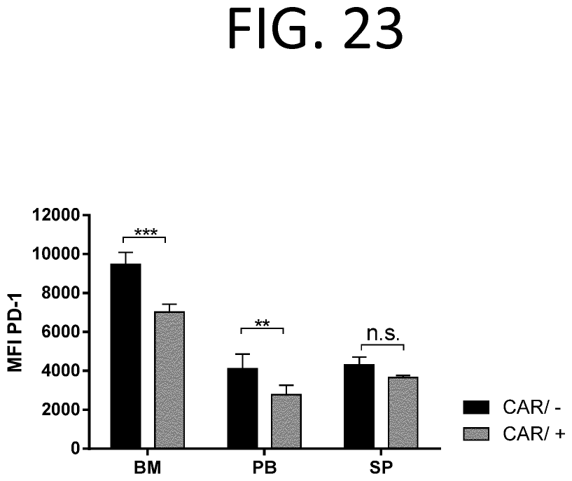

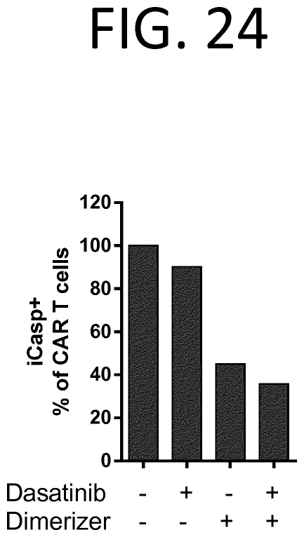

The construction of epHIV7 lentiviral vectors containing ROR1- or CD19-specific CARs with 4-1BB or CD28 costimulatory domain has been described, see reference [5], which is hereby incorporated by reference in its entirety for all purposes. A schematic design of the CAR constructs is provided in FIG. 1A-C. All vectors comprised a truncated epidermal growth factor receptor (EGFRt), see reference [9], which is hereby incorporated by reference in its entirety for all purposes, encoded in the transgene cassette downstream of the CAR. The CAR and EGFRt transgenes were separated by a T2A ribosomal skip element.

The inventors developed a reporter gene vector out of the epHIV7 lentiviral vector, containing wildtype green fluorescent protein (NFAT inducible GFPwt) or a GFP-variant destabilized by a mutated version of the residues 422 to 461 of mouse ornithine decarboxylase with an in vivo half-life of ^{˜}4 hours (NFAT inducible GFPd4) under control of a NFAT responsive element.

The inventors constructed an inducible suicide switch containing the iCasp9 suicide gene as described [21]

Preparation of Lentivirus

CAR/EGFRt, ffluc/GFP and NFATindGFP-encoding lentivirus supernatants were produced in 293T cells co-transfected with the respective lentiviral vector plasmids and the packaging vectors pCHGP-2, pCMV-Rev2 and pCMV-G using Calphos transfection reagent (Clontech, Mountain View, Calif.). Medium was changed 16 h after transfection, and lentivirus collected after 72 h. To collect virus particles, ultracentrifugation was performed at 24,900 rpm for 2 hours at 4° C. Jurkat cells were transduced with increasing amounts of virus to perform titration of lentivirus, and cells were analyzed for protein surface expression using flow cytometry on day 3 after transduction.

Preparation of CAR-T Cells

CAR-T cells were generated as described [5], [22]. In brief, CD8⁺ central memory and CD4⁺ bulk T cells were purified from PBMC of healthy donors using negative isolation with immunomagnetic beads (Miltenyi Biotec, Bergisch-Gladbach, Germany), activated with anti-CD3/CD28 beads according to the bead manufacturer's instructions (Life Technologies), and transduced with lentiviral supernatant at a moiety of infection (MOI) of 5. In some experiments, T cells were co-transduced with CAR/EGFRt and NFATindGFP-encoding lentiviral supernatant. Lentiviral transduction was performed on day 1 after bead stimulation by spinoculation. T cells were propagated and maintained in RPMI-1640 with 10% human serum, GlutaminMAX (Life technologies), 100 U/mL penicillin-streptomycin and 50 U/mL IL-2. Trypan blue staining was performed to quantify viable T cells. After bead removal on day 6 and expansion until day 10-14, T cells were enriched for EGFRt and further expanded using either a rapid expansion protocol (ROR1 CAR-T cells and corresponding untransduced control T cells) or antigen-specific expansion with irradiated CD19⁺ feeder cells (CD19 CAR-T cells and corresponding untransduced control T cells).

Analyses of CAR-T Cell Function

Cytotoxicity: Target cells were stably transduced with ffluc_GFP and incubated in triplicate wells at 1×10⁴ cells/well with effector T cells at an effector to target (E:T) ratio of 5:1. D-luciferin substrate (Biosynth, Staad, Switzerland) was added to the co-culture to a final concentration of 0.15 mg/ml and the decrease in luminescence signal in wells that contained target cells and T-cells was measured using a luminometer (Tecan, Männedorf, Switzerland). Specific lysis was calculated using the standard formula.

Cytokine secretion: 5×104 T-cells were plated in duplicate or triplicate wells with target cells at an E:T ratio of 4:1 (K562/ROR1, K562/CD19, RCH-ACV), and IFN-γ and IL-2 were measured by ELISA, or cytokine panels were measured by multiplex cytokine immunoassay (Luminex) in supernatant removed after 20-h incubation. Specific cytokine production was calculated by subtracting the amount of cytokines released by unstimulated CAR-T cells from the amount of cytokines released after antigen-specific stimulation. The remaining cytokine secretion in % as shown in the diagrams is normalized to CAR-T cells in the absence of dasatinib-treatment (100%).

Proliferation: T cells were labeled with 0.2 μM carboxyfluorescein succinimidyl ester (CFSE, Invitrogen), washed and plated in duplicate or triplicate wells with irradiated (80 Gy) stimulator cells at an E:T ratio of 4:1 (K562/ROR1, K562/CD19 or RCH-ACV). No exogenous cytokines were added to the culture medium. After a 72-hour incubation, cells were labeled with anti-CD3 mAb, and analyzed by flow cytometry to assess cell division of T cells. The proliferation index was calculated using FlowJo Software (FlowJO, LLC, Ashland, Oreg., USA), and used to determine the “remaining proliferation”, i.e. normalized to the prolfieration of CAR-T cells in the absence of dasatinib (100%).

Western Blot Analyses

After expansion, T cells were washed and cultured in absence of exogenous IL-2 for two days. Protein was isolated after a 30-minute stimulation of T cells with RCH-ACV (E:T ratio of 4:1). Western blots were performed under reducing conditions using the following antibodies according to the manufacturer's instructions: anti-pSrc fam Y416 (cell signaling #2101S), anti-Lck (cell signaling #2752S), anti-pCD247 Y142 (CD3zeta, BD #558402), anti-CD247 (Sigma Life science #HPA008750), anti-pZap70 Y319 (cell signaling #27175) and anti-Zap70 ( . . . ). Staining against β-actin was used as a loading control and for normalization. Western blots were developed using the ChemiDoc MP imaging system (Biorad, Munich, Germany); quantitative analysis of western blots was performed using Image Lab Software (Biorad, Munich, Germany).

NFAT Reporter Assay

T cells (co-)expressing the NFAT inducible GFPwt reporter gene were co-cultured in the presence of 10 U IL-2 with irradiated (80 Gy) Raji or K562 tumor cells at an E:T ratio of 5:1 or without target cells. T cells and target cells were co-cultured in the absence of dasatinib or in the presence of 100 nM dasatinib. After 24 hours of co-culture, cells were labeled with anti-CD3 mAb, and analyzed by flow cytometry to assess GFP expression in T cells.

Apoptosis Assays

CD8⁺ CD19 CAR-T cells were cultured in the presence of 50 U IL-2 either alone or with irradiated (80 Gy) K562/CD19 tumor cells at an E:T ratio of 4:1. Dasatinib was added to a final concentration of 100 nM either at the start of the assay or two hours after the start of the assay. After 24 hours of co-culture, co-cultures were labeled with anti-CD8 mAb, 7AAD and AnnexinV according to the manufacturer's instructions (BD Biosciences, Heidelberg, Germany), and analyzed by flow cytometry to evaluate the amount of apoptotic and dead T cells.

Elimination of ICasp+ T Cells

CAR-T cells co-expressing the iCasp suicide gene were cultured in the presence of 50 U/m IL-2, either without further treatment or in the presence of 100 nM dasatinib, and in the absence or presence of 10 nM AP20187, which is an iCaspase inducer drug. After 24 hours, cells were labeled with anti-CD3 mAB and analyzed by flow cytometry for the presence of iCasp+ T cells.

Preparation of Dasatinib

Lyophilized dasatinib was purchased from Selleck Chemicals (Houston, Tex., USA) and reconstituted in DMSO (AppliChem, Darmstadt, Germany) to obtain a stock solution with a concentration of 10 mM. Working solutions were prepared by further dilution in DMSO or medium as appropriate.

Preparation of Dexamethasone

Dexamethasone (SigmaAldrich, Steinheim, Germany) was reconstituted in DMSO (AppliChem, Darmstadt, Germany) to obtain a stock solution with a concentration of 100 mM. Working solutions were prepared by further dilution in DMSO or medium as appropriate.

Preparation of Other Tyrosine Kinase Inhibitors

Nilotinib, lapatinib and imatinib were purchased from Cell Signaling (Leiden, Netherlands) and reconstituted in DMSO (Sigma Aldrich) to obtain stock solutions with a concentration of 10 mM, respectively. Saracatinib, bosutinib and PP1-inhibitor were purchased from Selleck Chemicals (Houston, Tex., USA) and reconstituted in DMSO to obtain stock solutions with a concentration of 10 mM, respectively. Working solutions were prepared by further dilution in DMSO or medium as appropriate.

In Vivo Experiments

The Institutional Animal Care and Use Committee of UKW approved all mouse experiments. NOD.Cg-Prkdc^{scid} Il12rg^{tm1Wjl}/SzJ (NSG) mice (female, 6-8 week old) were purchased from Charles River (Sulzfeld, Germany). Mice were inoculated with 1×10⁶ Raji/ffluc_GFP tumor cells via tail vein injection (i.v.). Mice were treated with 5 ×10⁶ CAR-modified or control untransduced T-cells (CD4:CD8 ratio=1:1) via tail vein injection (i.v.). Dasatinib was administered by intraperitoneal injection (i.p.) at a dose of 10 mg/kg dasatinib (consecutive treatment), or with 5 mg/kg (intermittent treatment). Tumor burden and distribution was analyzed by serial bioluminescence imaging on an IVIS Lumina imager (Perkin Elmer, Baesweiler, Germany): mice received i.p. injections of 0.3 mg/g luciferin and images were acquired 10 minutes after luciferin injection in small binning mode at an acquisition time of 1 s to 1 min to obtain unsaturated images. Data were analyzed using LivingImage Software (Caliper) and the average radiance (or photon flux) analyzed in regions of interest that encompassed the entire body of each individual mouse. Mice were sacrificed at the end of the experiment and human T cells in bone marrow, peripheral blood and spleen were analyzed by flow cytometry. The presence of (human) cytokines in serum was measured using multiplex cytokine analysis.

### Example 2: Dasatinib Blocks CAR-T Cells Function

A) Dasatinib Blocks the Function of CD19 CAR-T Cells and ROR1 CAR-T Cells

Dasatinib Blocks the Cytolytic Activity of CD8⁺ CAR-T Cells

The inventors prepared CD8+ CAR-T cell lines from n=3 healthy donors. In each of the T-cell lines, the inventors enriched CAR-expressing T cells to >90% purity using the EGFRt-transduction marker. The inventors analyzed cytolytic activity of CD8⁺ CAR-T cells in a bioluminescence-based cytotoxicity assay using K562 that the inventors had transduced with either CD19 (for testing CD19 CAR-T cells) or ROR1 (for testing ROR1 CAR-T cells) as target cells. Dasatinib was added to the assay medium at the beginning of the assay.

The data show that dasatinib is capable of completely blocking cytolytic function of CD8+ T cells expressing a CD19 CAR with 4-1BB costimulation. The extent of the dasatinib-induced blockade of cytolytic CAR-T cell function is dose-dependent (FIG. 2A):
at concentrations 512.5 nM of dasatinib in the assay medium, the cytolytic function of CAR-T cells was not significantly affected (>88% specific lysis of target cells by treated CAR-T cells compared to 93% specific lysis of target cells by non-dasatinib treated CAR-T cells at t=12 h);
at a concentration of ≥25 nM of dasatinib in the assay medium, there was partial inhibition of the cytolytic function of CAR-T cells (26% specific lysis of target cells compared to 53% specific lysis by non-dasatinib treated CAR-T cells at t=6 h; and 73% specific lysis of target cells compared to 93% specific lysis of target cells by non-dasatinib treated CAR-T cells at t=12 h);
at a concentration of ≥50 nM of dasatinib in the assay medium, there was (near-) complete inhibition of the cytolytic function of CAR-T cells (less than 7% specific lysis of target cells up to t=6 h; and less than 12% specific lysis of target cells compared to 93% specific lysis of target cells by non-dasatinib treated CAR-T cells at t=12 h).

The inventors confirmed that dasatinib was capable of completely blocking cytolytic function of CD8+ T cells expressing a CD19 CAR with CD28 costimulatory domain (FIG. 2B).
at a concentration of ≥50 nM of dasatinib in the assay medium, there was partial inhibition of the cytolytic function of CAR-T cells (less than 23% specific lysis of target cells compared to 52% specific lysis of target cells by non-dasatinib treated CAR-T at t=5 h; and 47% residual specific lysis of target cells compared to 91% specific lysis of target cells by non-dasatinib treated CAR-T cells at t=10 h);
at a concentration of ≥100 nM of dasatinib in the assay medium, there was (near-) complete inhibition of the cytolytic function of CAR-T cells (less than 10% specific lysis of target cells for any given time point, compared to 91% specific lysis by non-dasatinib treated CAR-T cells t=10 h).

The inventors also confirmed that dasatinib was capable of completely blocking cytolytic function of CD8⁺ T cells expressing a ROR1 CAR with 4-1BB costimulatory domain (FIG. 2C).
at a concentration of 25 nM of dasatinib in the assay medium, there was less than 2% specific lysis of target cells compared to 73% specific lysis by non-dasatinib treated CAR-T cells up to t=5 h; and at a concentration of 50 nM of dasatinib in the assay medium, there was less than 2% specific lysis of target cells for any given timepoint, compared to 94% specific lysis by non-dasatinib treated CAR-T cells at t=10 h.

Dasatinib Blocks Cytokine Production and Secretion in CD8⁺ CAR-T Cells

The inventors analyzed the cytokine production and secretion of the CD8⁺ CAR-T cell lines in the presence or absence of dasatinib. CAR-T cells were co-cultured with K562 that the inventors had transduced with either CD19 (for testing CD19 CAR-T cells) or ROR1 (for testing ROR1 CAR-T cells). Dasatinib was added to the assay medium at the beginning of the co-culture assay. ELISA was performed to detect IFN-γ and IL-2 in supernatant removed from the co-culture.

The data show that dasatinib is capable of completely blocking cytokine production and secretion in CD8⁺ T cells expressing a CD19 CAR with 4-1BB costimulation. The extent of the dasatinib-induced blockade of cytokine production and secretion is dose-dependent (FIG. 3A):
at concentrations of ≥6.25 nM of dasatinib in the assay medium, there was less than 45% of residual specific IFN-γ production, and less than 60% of residual specific IL-2 production compared to non-dasatinib treated CAR-T cells;
at a concentration of ≥50 nM of dasatinib in the assay medium, there was no residual specific IFN-γ production, and less than 1% of residual specific IL-2 production compared to non-dasatinib treated CAR-T cells.

The inventors confirmed that dasatinib was capable of completely blocking cytokine production and secretion in CD8+ T cells expressing a CD19 CAR with CD28 costimulatory domain (FIG. 3B).
at a concentration of ≥50 nM of dasatinib in the assay medium, there was less than 4.5% of residual specific IFN-γ production, and less no residual specific IL-2 production compared to non-dasatinib treated CAR-T cells.

The inventors also confirmed that dasatinib was capable of completely blocking cytokine production and secretion in CD8+ T cells expressing a ROR1 CAR with 4-1BB costimulatory domain (FIG. 3C).
at a concentration of ≥50 nM of dasatinib in the assay medium, there was less than 3% of residual IFN-γ, and less than 8.5% of residual Il-2 production compared to non-dasatinib treated CAR-T cells.

These data are evidence for the fact that the dasatinib is a suitable inhibitor of cytokine secretion by CAR-T cells independent of receptor design and specificity.

Dasatinib Blocks Proliferation of CD8⁺ CAR-T Cells

The inventors analyzed the proliferation of CD8⁺ CAR-T cell lines in the presence or absence of dasatinib. CAR-T cells were labeled with CFSE and co-cultured with K562 that the inventors had transduced with either CD19 (for testing CD19 CAR-T cells) or ROR1 (for testing ROR1 CAR-T cells). Dasatinib was added to the assay medium at the beginning of the co-culture assay. Flow cytometric analyses were performed to determine the proliferation of T cells at the end of the co-culture assay. The proliferation index, indicating the average number of cell divisions performed during the assay period, was calculated, and was used to determine the remaining proliferation as normalized to the proliferation index of stimulated CAR-T cells in the absence of dasatinib as 100%.

The data show that dasatinib is capable of completely blocking the proliferation of CD8+ T cells expressing a CD19 CAR with 4-1BB costimulation. The extent of the dasatinib-induced blockade of proliferation is dose-dependent (FIG. 4A):
at concentrations of ≥3.125 nM of dasatinib in the assay medium, there was less than 80% of residual proliferation compared to non-dasatinib treated CAR-T cells;
at concentrations of ≥12.5 nM of dasatinib in the assay medium, there was less than 45% of residual proliferation compared to non-dasatinib treated CAR-T cells;
at a concentration of ≥50 nM of dasatinib in the assay medium, there was less than 8% of residual proliferation compared to non-dasatinib treated CAR-T cells.

The inventors confirmed that dasatinib was capable of completely blocking the proliferation of CD8⁺ T cells expressing a CD19 CAR with CD28 costimulatory domain (FIG. 4B).
at a concentration of ≥50 nM of dasatinib in the assay medium, there was less than 7% of residual proliferation compared to non-dasatinib treated CAR-T cells.

The inventors also confirmed that dasatinib was capable of completely blocking the proliferation of CD8⁺ T cells expressing a ROR1 CAR with 4-1BB costimulatory domain (FIG. 4C).
at a concentration of ≥50 nM of dasatinib in the assay medium, there was less than 7% of residual proliferation compared to non-dasatinib treated CAR-T cells. Stimulation with IL-2 was used as a positive control and reference.

Dasatinib Blocks Cytokine Production and Secretion in CD4⁺ CAR-T Cells

The inventors analyzed the cytokine production and secretion of the CD4⁺ CAR-T cell lines in the presence or absence of dasatinib. CAR-T cells were co-cultured with K562 that the inventors had transduced with CD19. Dasatinib was added to the assay medium at the beginning of the co-culture assay. A multiplex cytokine analysis was performed in supernatant removed from the co-culture.

The data show that dasatinib is capable of completely blocking cytokine production and secretion in CD4⁺ T cells expressing a CD19 CAR with 4-1BB costimulation. The extent of the dasatinib-induced blockade of cytokine production and secretion is dose-dependent (FIG. 5A):
at concentrations of ≥25 nM of dasatinib in the co-culture assay medium, the production and secretion of GM-CSF, IFN-γ, IL-2, IL-4, IL-5, IL-6 and IL-8 was (near-)completely blocked compared to non-dasatinib treated CAR-T cells (>95% reduction for GM-CSF, IFN-γ, IL-2, IL-4, IL-5, IL-6; IL-8).

The inventors confirmed that dasatinib was capable of completely blocking cytokine production and secretion in CD4⁺ T cells expressing a CD19 CAR with CD28 costimulatory domain (FIG. 5B).
at concentrations of ≥25 nM of dasatinib in the co-culture assay medium, the production and secretion of GM-CSF, IFN-γ, IL-2, IL-4, IL-5, IL-6 and IL-8 was (near-)completely blocked compared to non-dasatinib treated CAR-T cells (>95% reduction for GM-CSF, IFN-γ, IL-2, IL-4, IL-5, IL-6; IL-8).

B) Dasatinib Blocks the Function of SLAMF7 CAR-T Cells

Dasatinib Blocks the Cytolytic Activity of CD8⁺ SLAMF7 CAR-T Cells

The inventors prepared SLAMF7-specific CD8⁺ CAR-T cell lines from n=2 healthy donors. In each of the T-cell lines, the inventors enriched CAR-expressing T cells to >90% purity using the EGFRt-transduction marker. The inventors analyzed cytolytic activity of CD8⁺ CAR-T cells in a bioluminescence-based cytotoxicity assay using K562 that the inventors had transduced with SLAMF7 as target cells. K562-SLAMF7 had been generated by lentiviral transduction with the full length human SLAMF7 gene. Dasatinib was added to the assay medium at the beginning of the assay.

The data show that dasatinib is capable of completely blocking cytolytic function of CD8⁺ T cells expressing a SLAMF7-CAR with 4-1BB costimulation. The extent of the dasatinib-induced blockade of cytolytic CAR-T cell function is dose-dependent (FIG. 6A, upper diagram; see also FIG. 1D for the structure of the SLAMF7-CAR with 4-1BB costimulation):
at a concentration of ≥20 nM of dasatinib in the assay medium, there was partial inhibition of the cytolytic function of CAR-T cells (21% specific lysis of target cells compared to 63% specific lysis of target cells by non-dasatinib treated CAR-T cells at to t=6 h, and 35% specific lysis of target cells compared to 83% specific lysis of target cells by non-dasatinib treated CAR-T cells at t=12 h);
at a concentration of >40 nM of dasatinib in the assay medium, there was (near-) complete inhibition of the cytolytic function of CAR-T cells (less than 5.5% specific lysis of target cells up to t=6 h; and less than 10% specific lysis of target cells compared to 83% specific lysis of target cells by non-dasatinib treated CAR-T cells at t=12 h).

The inventors confirmed that dasatinib was also capable of completely blocking cytolytic function of CD8⁺ T cells expressing a SLAMF7 CAR with CD28 costimulatory domain (FIG. 6A, lower panel; see also FIG. 1E for the structure of the SLAMF7 CAR with CD28 costimulatory domain).
at a concentration of ≥20, 40 and 60 nM of dasatinib in the assay medium, there was partial inhibition of the cytolytic function of CAR-T cells (less than 21% specific lysis of target cells compared to 67% specific lysis of target cells by non-dasatinib treated CAR-T at t=6 h; and less than 35% residual specific lysis of target cells compared to 85% specific lysis of target cells by non-dasatinib treated CAR-T cells at t=12 h);
at a concentration of ≥80 nM of dasatinib in the assay medium, there was (near-)complete inhibition of the cytolytic function of CAR-T cells (less than 3% specific lysis of target cells for any given time point, compared to 85% specific lysis by non-dasatinib treated CAR-T cells t=12 h).

Dasatinib Blocks Cytokine Production and Secretion in CD8⁺ and CD4⁺ SLAMF7 CAR-T Cells

The inventors prepared SLAMF7-specific CD8⁺ and CD4⁺ CAR-T cell lines from n=2 healthy donors. In each of the T-cell lines, the inventors enriched CAR-expressing T cells to >90% purity using the EGFRt-transduction marker. The inventors analyzed the cytokine production and secretion of CD8⁺ and CD4⁺ CAR-T cell lines in the presence or absence of dasatinib. CAR-T cells were co-cultured with K562 that the inventors had transduced with SLAMF7. Dasatinib was added to the assay medium at the beginning of the co-culture assay. ELISA was performed to detect IFN-γ and IL-2 in supernatant removed from the co-culture.

The data show that dasatinib is capable of completely blocking cytokine production and secretion in CD8⁺ T cells expressing a SLAMF7 CAR with 4-16B costimulation. The extent of the dasatinib-induced blockade of cytokine production and secretion is dose-dependent (FIG. 6B):
at concentrations of ≥20 nM of dasatinib in the assay medium, there was less than 15% of residual specific IFN-γ production, and no residual specific IL-2 production compared to non-dasatinib treated CAR-T cells;
at a concentration of ≥40 nM of dasatinib in the assay medium, there was less than 0.8% residual specific IFN-γ production, and no residual specific IL-2 production compared to non-dasatinib treated CAR-T cells.

The inventors confirmed that dasatinib was capable of completely blocking cytokine production and secretion in CD8⁺ T cells expressing a SLAMF7 CAR with CD28 costimulatory domain (FIG. 6B).
at a concentration of ≥20 nM of dasatinib in the assay medium, there was less than 4% of residual specific IFN-γ production, and less no residual specific IL-2 production compared to non-dasatinib treated CAR-T cells.
at a concentration of ≥40 nM of dasatinib in the assay medium, there was less than 0.2% residual specific IFN-γ production, and no residual specific IL-2 production compared to non-dasatinib treated CAR-T cells.

The inventors also confirmed that dasatinib was capable of completely blocking cytokine production and secretion in CD4⁺ T cells expressing a SLAMF7 CAR with 4-1BB costimulatory domain (FIG. 6C).
at a concentration of ≥20 nM of dasatinib in the assay medium, there was less than 0.5% of residual IFN-γ, and no residual 11-2 production compared to non-dasatinib treated CAR-T cells.

The inventors also confirmed that dasatinib was capable of completely blocking cytokine production and secretion in CD4⁺ T cells expressing a SLAMF7 CAR with CD28 costimulatory domain (FIG. 6C).
at a concentration of ≥20 nM of dasatinib in the assay medium, there was less than 1.2% of residual IFN-γ, and no residual Il-2 production compared to non-dasatinib treated CAR-T cells.

These data are additional evidence for the fact that the dasatinib is a suitable inhibitor of cytokine secretion by CAR-T cells independent of receptor design and specificity.

C) Dasatinib Blocks CAR-T Cell Signaling

Dasatinib Blocks Phosphorylation of Kinases Involved in CAR-Signaling

The inventors co-cultured CD8⁺ T cells expressing a CD19 CAR with 4-1BB costimulation with RCH-ACV target cells (CD19+) in the presence or absence of 100 nM dasatinib, and performed Western blot analyses to determine the phosphorylation state of kinases presumably involved in CAR-signaling.

In CAR-T cells that the inventors had co-cultured in the presence of dasatinib, the phosphorylation of Lck/Src family kinase at tyrosine 416, CAR CD3 zeta at tyrosine 142, and ZAP70 at tyrosine 319 was lower compared to CAR-T cells that the inventors had co-cultured in the absence of dasatinib (FIG. 7A). For reference and control the inventors performed concomitant Western blots for Lck, CAR CD3 zeta, and ZAP70, and β-actin, both in dasatinib-treated and non-treated CAR-T cells. The CD3 zeta domain comprised in the CAR (CAR CD3 zeta) was distinguished from endogenous CD3 zeta by its distinct molecular weight.

Quantitative Western blot analysis showed that phosphorylation in dasatinib-treated CAR-T cells was only 12.86% (CAR CD3 zeta), 21.57% (Lck) and 11.61% (ZAP70), respectively compared to CAR-T cells co-cultured in the absence of dasatinib (FIG. 7B).

Dasatinib Blocks NFAT Mediated Induction of GFP Expression

The inventors prepared CD8⁺ CD19 CAR/4-1BB T cells, that the inventors transduced to co-express an NFAT-inducible GFP reporter gene. The inventors co-cultured these T cells with Raji (CD19⁺) or K562 (CD19) tumor cells, either in the presence or absence of 100 nM dasatinib, and performed flow cytometric analyses to determine expression of the GFP reporter gene.

The data show that in the presence of dasatinib, induction of GFP reporter gene expression was completely abrogated. The mean fluorescence intensity (MFI) of GFP expression in the absence of dasatinib was on average 1211 after stimulation with Raji; and was only 129 in the presence of dasatinib after stimulation with Raji, which is similar to the background MFI obtained with unstimulated T cells (MFI 117) (FIG. 8, left panel).

The inventors confirmed that the presence of dasatinib abrogated NFAT signaling and GFP reporter gene expression in CD4⁺ CD19 CAR/4-1BB T cells (FIG. 8, right panel).

The data show that dasatinib completely blocks CAR signaling and prevents expression of the NFAT transcription factor in both CD8⁺ and CD4⁺ T cells.

Interruption of Signaling by Dasatinib does not Decrease the Viability of CAR-T Cells

The inventors cultured CD8⁺ CD19 CAR/4-1BB T cells alone or in co-culture with irradiated K562/CD19 for 24 hours, either in the absence or presence of 100 nM dasatinib. At the end of the co-culture, the inventors performed staining with Annexin-V and 7-AAD to determine the percentage of live CAR-T cells (Annexin-V-negative/7-AAD-negative), CAR-T cells undergoing apoptosis (Annexin-V-positive/7-AAD-negative), and dead CAR-T cells (Annexin-V-positive/7-AAD-positive).

The data show that after stimulation with K562/CD19 tumor cells and in presence of dasatinib, there was a higher proportion of live CAR-T cells and smaller proportion of dead or apoptotic CAR-T cells (alive: 47.4%; apoptotic: 45.7%; dead 6.9%) than in the absence of dasatinib (alive: 25.4%; apoptotic: 66.2%; dead 8.4%) (FIG. 9). A similar effect was observed when dasatinib was added to the co-culture of CAR-T cells and K562/CD19 tumor cells at 2 hours after the start of the co-culture assay (alive: 41.7%; apoptotic: 51.3%; dead 7%). These data show that dasatinib can protect CAR-T cells from activation induced cell death (AICD) after encountering tumor cells.

In aggregate, the data show that dasatinib is able to completely block the stimulation, activation and subsequent effector function of resting CAR-T cells. The blockade is effective in both CD8+ and CD4+ T cells, and works independent from antigen-specificity and particular design (example: costimulatory moiety) of the CAR construct. The blockade of CAR-T cell function by dasatinib is dose-dependent. Partial inhibition of CAR-T cell function can also be accomplished, and is dependent on the selected concentration of dasatinib.

### Example 3: Dasatinib Blocks the Function of Activated CAR-T Cells

Dasatinib Blocks the Function of Activated CAR-T Cells

The inventors sought to determine whether dasatinib was able to block the function of CAR-T cells that are already activated and in the process of executing their effector function. The inventors prepared CD8⁺ CAR-T cell lines from n=3 healthy donors. In each of the T-cell lines, the inventors enriched CAR-expressing T cells to >90% purity using the EGFRt-transduction marker. The inventors analyzed cytolytic activity of CD8⁺ CAR-T cells in a bioluminescence-based cytotoxicity assay using K562 target cells that the inventors had transduced with CD19. Dasatinib was added to the assay medium 1 hour after the start of the co-culture (dasa+1 h). For comparison, the inventors included a setting where dasatinib was added right at the start of the co-culture (dasa; as was done in experiments in Example 2), and a setting where no dasatinib was added (untreated).

The data show that dasatinib is capable of blocking the cytolytic function of already activated CD8⁺ T cells expressing a CD19 CAR with 4-1BB costimulation. In the setting where dasatinib (100 nM) was added to the assay medium 1 hour after the start of the co-culture, the inventors detected a reduced increase in the percentage of specifically lysed target cells for up to 7 hours of the co-culture. After 7 hours, the percentage of specifically lysed target cells plateaued and did not increase further, with a specific lysis of 34% at t=10 hours (FIG. 10A). For comparison, in the setting were no dasatinib was added to the co-culture, there was a much faster and steady increase in specific target cell lysis over the entire 10-hour assay period. At each of the analysis time points beyond 2 hours, the percentage of specifically lysed target cells was higher compared to the setting with delayed (+1 hour) dasatinib addition. At the 10-hour analysis time point, the percentage of specifically lysed target cells was >90% (FIG. 10A). In the setting where dasatinib (100 nM) was added at the start of the co-culture, there was a complete blockade of cytolytic activity, consistent with the data obtained in Example 2.

The inventors also show that dasatinib is capable of blocking cytokine production and secretion of already activated CD8⁺ T cells expressing a CD19 CAR with 4-1BB costimulation. CD8⁺ CAR-T cells were co-cultured with K562/CD19 target cells for 20 hours, and the presence of IFN-γ and IL-2 in supernatant obtained from these co-cultures analyzed by ELISA. The data show that in the setting where dasatinib (100 nM) was added to the assay medium at 2 hours after the start of the co-culture, there were lower levels of IFN-γ and IL-2 compared to the setting where no dasatinib was added (untreated control) (FIG. 10B). After normalization (level of cytokine production in untreated CAR-T cells =100%), the percentage of residual IFN-γ and Il-2 production was 51% and 28%, respectively (FIG. 10B).

The inventors also show that dasatinib is capable of blocking the proliferation of already activated CD8⁺ T cells expressing a CD19 CAR with 4-1BB costimulation. CD8⁺ CAR-T cells were labeled with CFSE and co-cultured with K562/CD19 target cells. The proliferation of CAR-T cells was analyzed after 72 hours based on CFSE dye dilution, and the proliferation index calculated. Dasatinib (100 nM) was added either at the start of the co-culture (0 h), or 1 hour (+1 h), or 3 hours (+3 h), or 48 hours (+48 h) after the start of the co-culture. The proliferation observed in CAR-T cells that were stimulated with K562/CD19 target cells in the absence of dasatinib was used as a reference (proliferation=100%). The data show that the addition of dasatinib at 1 hour and at 3 hours after the start of the co-culture led to lower proliferation index of less than 26% and 72% compared to untreated CAR-T cells (FIG. 10C). The addition of dasatinib at 48 hours after the start of the co-culture led to a lower proliferation index of 91% compared to untreated CAR-T cells; however, this difference was not statistically significant (FIG. 10C).

In aggregate, these data show that dasatinib is able to block the function of CAR-T cells that are already activated and are in the process of executing their effector functions. This ability is of particular clinical relevance for mitigating toxicity or preventing the exacerbation of toxicity in the context of CAR-T cell immunotherapy.

### Example 4: Dasatinib Prevents CAR-T Cell Activation During Sequential Stimulation

The inventors employed the NFAT/GFP reporter system to interrogate the effects of dasatinib on activated CAR-T cells on a signaling level and to evaluate if dasatinib mediated inhibition could be sustained over time and during sequential antigen encounter (FIG. 11). NFAT reporter CAR T cells were generated as described in example 1.

The inventors analyzed the NFAT-driven expression of GFP in CD8⁺ and CD4⁺ CAR-T after co-culture with K562 target cells that the inventors had transduced with CD19. Dasatinib was added to the assay medium 1 hour after the start of the co-culture (dasa+1 h) or during assay set up (dasa). For comparison, the inventors included a setting where no dasatinib was added (untreated). Subsequently, target cells and 100 nM dasatinib were added simultaneously every 24 hours.

The data show that on day 1, T cells were partially activated and showed reduced expression of GFP when dasatinib was added one hour after assay set up (MFI of 734 compared to 1949 in untreated but stimulated CAR-T cells). When dasatinib was present from the beginning, expression of GFP was completely suppressed on day 1 (MFI 137).

The data show that once dasatinib was present, GFP was not induced by subsequent stimulation on day 2 or 3, neither in T cells that had been treated from the beginning, nor T cells that had been treated at 1 hour after assay set up. Instead, GFP levels decreased until day 3, indicating that further antigen specific stimulation was prevented by dasatinib and cells were maintained in a function OFF state.

The inventors confirmed that dasatinib prevents subsequent antigen specific stimulation in CD4⁺ T cells co-expressing the CD19 CAR with 4-1BB costimulatory domain and the NFAT/GFP reporter system (FIG. 11).

The data show that on day 1, T cells were partially activated and showed reduced expression of GFP when dasatinib was added one hour after assay set up (MFI of 841 compared to 2288 in untreated but stimulated CAR-T cells). When dasatinib was present from the beginning, expression of GFP was completely suppressed on day 1 (MFI 317).

The data show that once dasatinib was present, GFP was not induced by subsequent stimulation on day 2 or 3, neither in T cells that had been treated from the beginning, nor T cells that had been treated at 1 hour after assay set up. Instead, GFP levels decreased until day 3 (MFI 108 and MFI 326, respectively), while GFP expression remained high in untreated CAR_T cells (MFI 2499), indicating that further antigen specific stimulation was prevented by dasatinib and cells were maintained in a function OFF state.

In aggregate, these data confirm that dasatinib is able to block the function of CAR-T cells that are already activated. Furthermore, the data show that dasatinib can interrupt already induced activation, and prevents the subsequent induction of transcription factors despite presence of antigen.

### Example 5: The Blockade of CAR-T Cell Function is Rapidly and Completely Reversible after Removal of Dasatinib

The Blockade of CAR-T Cell Function is Rapidly and Completely Reversible after Short-Term Exposure to Dasatinib

The inventors prepared CD8⁺ T-cell lines expressing a CD19 CAR with 4-1BB costimulation from n=3 healthy donors. In each of the T-cell lines, the inventors enriched CAR-expressing T cells to >90% purity using the EGFRt-transduction marker. The inventors analyzed cytolytic activity of CD8⁺ CAR-T cells in a bioluminescence-based cytotoxicity assay using K562 target cells that the inventors had transduced with CD19.

Dasatinib (100 nM) was added to the assay medium at the start of the co-culture (t=−2 h). After 2 hours (t=0 h), the assay medium was discarded and replaced with fresh assay medium (i.e. dasatinib was removed). The CAR-T cell cytolytic activity was analyzed at 1-hour intervals for 10 hours. For comparison, the inventors included a setting where no dasatinib was present in the assay medium (FIG. 12A).

The data show that in the presence of dasatinib (i.e. in the first 2 hours of the co-culture assay), CAR-T cells did not exert any cytolytic activity, consistent with the data obtained in Example 2. However, immediately after the medium change (i.e., immediately after removal of dasatinib), CAR-T cells started to exert their cytolytic activity. At +4 hours, CAR-T cells had conferred 77% specific lysis of target cells. At the end of the co-culture assay at 10 hours, CAR-T cells had conferred >95% specific lysis of target cells, similar to CAR-T cells that had not been treated with dasatinib in the first 2 hours of the co-culture (FIG. 12A). The data were confirmed with CD8⁺ T-cell lines expressing a CD19 CAR with CD28 costimulation that the inventors prepared from n=2 healthy donors (FIG. 12B).

Long-Term Exposure to Dasatinib does not Decrease the Viability of CAR-T Cells

CD8⁺ CD19 CAR/4-1BB-T cells were maintained in culture medium supplemented with 50 U/ml IL-2, either in the absence of dasatinib [(−)] or in the presence of dasatinib [100 nM,(+)] for eight consecutive days. Dasatinib was added to the culture medium every 24 hours.

On day 2 days (i.e. 48 hours, short-term exposure) and on day 8 (long-term exposure), the inventors obtained an aliquot of CAR-T cells from each culture condition and determined cell viability using staining with 7AAD and AnnexinV. At each time point, the percentage of viable CAR-T cells was higher in CAR-T cell lines that had been maintained in presence of dasatinib when compared to CAR-T-cells that had been cultured without dasatinib (FIG. 13) The data show that both short-term and long-term exposure to dasatinib does not lead to decreased viability of CAR-T cells.

The Blockade of CAR-T Cell Unction is Rapidly and Completely Reversible after Exposure to and Subsequent Removal of Dasatinib

CD8⁺ CD19 CAR/4-1BB-T cells were maintained in culture medium supplemented with 50 U/ml IL-2, either in the absence of dasatinib or in the presence of dasatinib (100 nM) for seven consecutive days. Dasatinib was added to the culture medium every 24 hours.

After 1 day (i.e. 24 hours, short-term exposure) and after 7 days (long-term exposure), the inventors obtained an aliquot of CAR-T cells from each culture condition and performed a complete medium change to remove dasatinib. Then, the inventors performed functional testing to assess whether the prior exposure to dasatinib had an influence on the subsequent ability of CAR-T cells to exert their antitumor function. The data show that both after short-term and long-term exposure to dasatinib and subsequent removal of dasatinib, CAR-T cells were able to exert their antitumor functions, at a level and with a potency that was identical to CAR-T cells that had been cultured in the absence of dasatinib.

The data in FIG. 14A show that after 1-day (left diagram) and after 7-day exposure to dasatinib (right diagram), and subsequent removal of dasatinib, CAR-T cells exerted rapid and potent specific cytolytic activity of target cells, equivalent to CAR-T cells that had never been exposed to dasatinib (dasa/no dasa compared to no dasa/no dasa). The data in FIG. 14A also show that after 1-day (left diagram) and after 7-day exposure to dasatinib (right diagram), subsequent removal of dasatinib (washing) and re-newed exposure to dasatinib (dasa/dasa), the complete blockade of CAR-T cell cytolytic activity was still working.

The data in FIG. 14B show that after 1-day and after 7-day exposure to dasatinib, and subsequent removal of dasatinib, CAR-T cells produced and secreted IFN-γ (left diagram) and IL-2 (right diagram) in response to stimulation with target cells, equivalent to CAR-T cells that had never been exposed to dasatinib (Dasa pre −). The data in FIG. 14B also show that after 1-day and after 7-day exposure to dasatinib, subsequent removal of dasatinib and re-newed exposure to dasatinib (Dasa during), the complete blockade of CAR-T cell cytokine production and secretion was still working.

The data in FIG. 14C show that after 1-day and after 7-day exposure to dasatinib and subsequent removal of dasatinib, CAR-T cells proliferated in response to stimulation with target cells, equivalent to CAR-T cells that had never been exposed to dasatinib (Dasa pre −). The data in FIG. 14C also show that after 1-day and after 7-day exposure to dasatinib, subsequent removal of dasatinib and re-newed exposure to dasatinib (Dasa during), the complete blockade of CAR-T cell proliferation was still working.

In aggregate, these data show that the blockade of CAR-T cell function by dasatinib does not negatively affect CAR-T cells viability, and that the blockade of CAR-T cell function is rapidly and completely reversible after removal of dasatinib independent of the duration of pre-treatment.

Previous exposure to dasatinib does not preclude the ability of dasatinib to block CAR-T cell function upon repeated exposure. These data show that dasatinib can be used to precisely and very effectively control the function of CAR-T cells.

### Example 6: Dasatinib Blocks CAR-T Cell Function In Vivo and Prevents Cytokine Release Syndrome

Dasatinib Blocks CAR-T Cell Function in a Murine Xenograft Lymphoma Model

The inventors employed a xenograft model in immunodeficient mice (NSG/Raji) to assess the influence of dasatinib on CD19 CAR/4-1BB-T cells in vivo. The experiment setup and treatment schedule is provided in FIG. 15A. In brief, cohorts of n3 mice were inoculated with 1×10\hat{ }6 firefly-luciferase_GFP-transduced Raji tumor cells on day 0, and on day 7 mice were treated with either CAR-transduced or control untransduced T cells. T-cell products consisted of equal proportions of CD4⁺ and CD8 T cells (1:1 ratio), the total dose was 5×10\hat{ }6 T cells. In each treatment cohort, a subgroup of mice received dasatinib beginning 3 hours prior to T-cell transfer, and then every 6 hours for a total of 6 doses.

Based on the known pharmacokinetic and—dynamic of dasatinib in mice [23] (assuming that pharmacokinetics after i.p. injection will not be faster than after i.v. injection) this provided a window between 3 hours prior to T-cell transfer and 33 hours after T-cell transfer (total window: 36 hours) where dasatinib was present in mouse serum at a concentration of at least 100 nM. In this mouse model, blockade by dasatinib should therefore be effective until day +1 after CAR-T cell transfer, and not be effective anymore on day +3 after CAR-T cell transfer.

Dasatinib blocks cytokine production and secretion in CAR-T cells in vivo and prevents cytokine release

The inventors analyzed serum cytokine levels in mice (NSG/Raji) that had been concurrently treated with CAR-T cells and dasatinib. To determine cytokine levels, the inventors performed multiplex cytokine analysis in mouse serum (FIG. 15B).

The data show that in mice that had received CAR-T cells and dasatinib (day +1, CAR/+), there were significantly lower serum levels of GM-CSF (6.4 pg/ml), IFN-γ (13.4 pg/ml), TNF-α (0.04 pg/ml), IL-2 (below detection limit), IL-5 (21.4 pg/ml) and IL-6 (below detection) [i.e. 3.2% of the GM-CSF, 1.7% of the IFN-γ, 0.3% of the TNF-α and 2.6% of the IL-5 level] compared to mice the had received CAR-T cells and no dasatinib (CAR/−) (FIG. 15B). The data confirm the inventors' prior observation in vitro, that dasatinib is able to block cytokine secretion of CAR-T cells (see Example 2). The data also confirm the inventors' prior observation in vitro, that the blockade of CAR-T cell function by dasatinib is rapidly reversible (see Example 5) (FIG. 15B).

On day +3 of the experiment, when dasatinib had been discontinued, cytokine serum levels had increased to 45.8 pg/m GM-CSF, 411.8 pg/ml IFN-γ, 0.9 pg/m TNF-α, 0.2 pg/ml IL-2, 331.2 pg/ml IL-5 and 0.9 pg/ml IL-6 [which is a fold-change of 7.2 in GM-CSF, 30.7 in IFN-γ, 22.9 in TNF-α and 15.5 in IL-5 secretion, respectively] compared to serum cytokine levels observed in the same mice on day +1 (when dasatinib had been administered) (FIG. 15).

In aggregate, these data show that i) the cytokine production and secretion in CAR-T cells can be blocked by dasatinib in vivo; ii) that the blockade of cytokine production and secretion can be maintained by repeated administration of dasatinib for at least 36 hours; iii) that the blockade of cytokine secretion is reversible after discontinuation of dasatinib in vivo.

CAR-T Cell Function is Blocked in the Presence of Dasatinib In Vivo/CAR-T Cells Resume their

Antitumor Function In Vivo Once Exposure to Dasatinib is Discontinued The inventors analyzed the CAR-T cell antitumor function in mice that had received either CAR-T cells or untransduced control T cells, and had either received dasatinib according to the treatment schedule in FIG. 15A, or had not received dasatinib. Raji tumor burden was determined by bioluminescence imaging on day −1, day 1 and day 3.

The data show that between day −1 and day 1, mice that received CAR-T cells plus dasatinib showed tumor progression at a similar rate (CAR/+; 14.1fold change) as mice that had received untransduced control T cells and dasatinib (ctrl/+; 15.4 fold change) (FIG. 15C, black bars), i.e. the CAR was ineffective. For comparison, tumor progression was significantly slower in this short interval in mice that had received CAR-T cells without dasatinib.

The data show that between day +1 and +3, when dasatinib administration had been discontinued, there was a strong reduction in tumor burden in both groups that had been treated with CAR-T cells (with or without prior dasatinib), in particular there was a stron reduction in tumor burden in mice that had been previously treated with dasatinib, illustrating that the blockade of CAR-T function by dasatinib was rapidly reversible in vivo (FIG. 15C, grey bars).

The inventors analyzed the presence of human T cells in bone marrow (BM), spleen (SP) and peripheral blood (PB) of mice, at d1 and d3 after T cell injection using flow cytometry. Live human T cells were identified as 7AAD⁻, CD3⁺ and CD4⁺.

The data show that on day 1, the frequency of human T cells were not different in mice that received CAR-T cells and dasatinib (CAR/treated: BM: 0.087%; PB: 0.19%) compared to mice that received CAR-T cells and no dasatinib (CAR/untreated: BM: 0.099%; PB 0.16%); i.e. the administration of dasatinib did not impair the engraftment of CAR-T cells (FIG. 15D, d+1). On day +3, the frequency of CAR-T cells was lower in mice that had been concurrently treated with dasatinib (CAR/treated: BM: 0.23%; PB: 0.36%) compared to mice that had not received dasatinib (CAR/untreated: BM: 0.56%; PB: 0.61%) (FIG. 15D, d+3), consistent with the inventors' observation in vitro, that dasatinib was capable of blocking CAR-T cell proliferation and expansion.

Dasatinib Blocks CAR-Signaling and Induction of the NFAT Transcription Factor in CAR-T Cells In Vivo

The inventors prepared CD8⁺ and CD4⁺ CD19CAR/41BB T cells, which the inventors transduced to co-express an NFAT inducible GFP reporter gene. The inventors used a xenograft mouse model as described above (FIG. 15A), and performed flow cytometry analyses to determine the expression of the GFP reporter gene in human T cells isolated from bone marrow and spleen of mice treated with either CAR-T cells or control T cells in the presence or absence of dasatinib, and during (d+1) or after (d+3) dasatinib treatment.

The data show that in the presence of dasatinib, expression of GFP reporter gene in CAR-T cells obtained from bone marrow and spleen was significantly lower in mice that had been treated with dasatinib compared to mice that had not been treated with dasatinib (FIG. 15E). The mean fluorescence intensity (MFI) for GFP in bone marrow CAR-T cells was 10687 in the absence of dasatinib (CAR/untreated) on d+1, and was only 6967 in the presence of dasatinib (CAR/treated), which is a reduction of 35%. A similar reduction of GFP reporter gene expression was observed in spleen CAR-T cells on d+1 (reduction: 36%). On d+3, when dasatinib was not in effect any more, the difference was only 10% between CAR-T cells that had been previously treated and untreated CAR-T cells in the bone marrow, and only 23% between previously treated and untreated CAR-T cells in the spleen.

In aggregate, these data show that dasatinib is capable of controlling the function of CAR-T cells in vivo. In particular, the data show that administration of dasatinib prevents cytokine release from CAR-T cells and prevents cytokine release syndrome. Treatment with dasatinib does not impair the engraftment of T cells. Once exposure to dasatinib is discontinued, CAR-T cells resume their antitumor function.

### Example 7: Dasatinib Blocks the Function of Activated CD19CAR/4-1BB CAR-T Cells In Vivo

Dasatinib Blocks CAR-T Cell Function an Murine Xenograft Lymphoma Model

The inventors employed a xenograft model in immunodeficient mice (NSG/Rai) to assess the influence of dasatinib on activated CAR/4-1BB-T cells in vivo. The experiment set up and treatment schedule is provided in FIG. 16A. In brief, cohorts of n6 mice were inoculated with 1×10\hat{ }6 firefly-luciferase GFP-transduced Raji tumor cells on day 0, and on day 7 mice were treated with either CAR-transduced or control untransduced T cells. T-cell products consisted of equal proportions of CD4⁺ and CD8⁺ T cells 1:1 ratio the total dose was 5×10\hat{ }6 T cells. In indicated cohorts, mice received dasatinib three days after T-cell transfer, and then every 6 hours for a total of 8 doses. Based on the known pharmacokinetic and—dynamic of dasatinib in mice this provided a window between day 10 and day 12 after tumor inoculation where dasatinib was present in mouse serum at a concentration above the threshold required for blocking the function of CAR-T cells. CAR-T cell function is OFF in the presence of dasatinib, and re-ignites to function ON once dasatinib administration is discontinued.

The inventors analyzed the CAR-T cell antitumor function in mice that had received either CAR-T cells or untransduced control T cells, and had either received dasatinib according to the treatment schedule in FIG. 16A, or had not received dasatinib. Raji tumor burden was determined by bioluminescence imaging on day 7, day 10, day 12, day 14, day 17, and subsequently, once a week (FIG. 16B). The data show that in the first phase after T-cell transfer (day 7 to day 10), CD19-CAR T-cells commenced exerting their antilymphoma activity and delayed lymphoma progression as demonstrated by BLI (FIG. 16B). In the second phase after T-cell transfer (day 10 to day 12), dasatinib rapidly induced a function OFF state and halted antilymphoma reactivity, as evidenced by strongly increasing BLI signal in the dasatinib treatment cohort. In contrast, the BLI signal did not increase during this phase in mice that had received CD19-CAR T-cells but no dasatinib. In the third phase (after day 12), administration of dasatinib was discontinued in order to allow CAR T-cells to revert back into their function ON state. CAR T-cells rapidly resumed their antilymphoma function as revealed by rapidly decreasing BLI signal. Following day 17, CAR-T cells were even more effective in controlling the tumor in cohorts that had been treated with dasatinib, as the tumor was controlled in all mice until day 59. In contrast, tumor relapsed in the majority of mice in the cohort that had received CAR-T cells and no dasatinib (FIG. 16B)

The data show that between day 7 and day 10, mice that received CAR-T cells showed a reduced tumor growth that was equal in mice receiving CAR only and mice that were determined to receive dasatinib subsequently (growth rate of 298% and 227%, respectively) when compared to mice receiving control T cells (growth rate of 2018%). Between day 10 and day 12, mice that received CAR-T cells plus dasatinib showed tumor progression at a much higher rate (CAR (ON/OFF/ON)); 405%) as mice that had received CAR-T cells alone (CAR(ON) 22.2%) (FIG. 16C), i.e. the CAR was ineffective in the presence of dasatinib despite primary activation of CAR-T cells. The data show that between day 12 and 17, when dasatinib administration had been discontinued, there was a strong reduction in tumor burden in both groups that had been treated with CAR-T cells (with or without prior dasatinib; reduction of tumor luminescence by 66% and 97.8%, respectively), in particular there was a strong reduction in tumor burden in mice that had been previously treated with dasatinib, illustrating that the blockade of CAR-T function by dasatinib was rapidly reversible in vivo (FIG. 16C).

Dasatinib Blocks Cytokine Production and Secretion from CAR-T Cells In Vivo and Prevents Cytokine Release Syndrome

The inventors analyzed serum cytokine levels in mice (NSG/Raji) that had been concurrently treated with CAR-T cells and dasatinib. To evaluate the expression of cytokines, the inventors performed analysis of IFNγ in mouse serum (FIG. 16D).

The data show that in mice that had received CAR-T cells, IFN γ serum levels were equal on day 10, thus before dasatinib administration. On day 12, thus after dasatinib administration, there were significantly lower serum levels of IFN-γ (24 pg/ml) in mice that had received dasatinib (CAR(ON/OFF/ON)) compared to mice the had received CAR-T cells and no dasatinib (CAR(ON)) (157 pg/ml, FIG. 16D). The data confirm the inventors' prior observation in vitro, that dasatinib is able to block cytokine secretion of activated CAR-T cells, and prevents the subsequent stimulation of inhibited T cells (see Example 3 and Example 4).

The data also confirm the inventors' prior observation in vitro, that the blockade of CAR-T cell function by dasatinib is rapidly reversible (see Example 5). On day 14 of the experiment, when dasatinib had been discontinued, cytokine serum levels had increased to 38 pg/m IFN-γ, which is a fold-change of 1.6 compared to serum cytokine levels observed in the same mice on day 12 (when dasatinib had been administered) (FIG. 16D).

In aggregate, these data show that i) the cytokine production and secretion in activated CAR-T cells can be blocked by dasatinib in vivo; ii) that the blockade of cytokine production and secretion can be maintained by repeated administration of dasatinib for at least 54 hours; iii) that the blockade of cytokine secretion is reversible after discontinuation of dasatinib in vivo.

### Example 8: Dasatinib Blocks the Function of Activated CD19/CD28 CAR-T Cells In Vivo

Dasatinib Blocks CAR-T Cell Unction an Murine Xenograt Lymphoma Model

The inventors employed a xenograft model in immunodeficient mice (NSG/Raji) to assess the influence of dasatinib on activated CAR/CD28-T cells in vivo. The experiment schedule set up and treatment schedule is provided in FIG. 17A. In brief, cohorts of n>8 mice were inoculated with 1×10\hat{ }6 firefly-luciferase_GFP-transduced Raji tumor cells on day 0, and on day 7 mice were treated with either CAR-transduced or control untransduced T cells. T-cell products consisted of equal proportions of CD4⁺ and CD8⁺ T cells (1:1 ratio), the total dose was 5×10\hat{ }6 T cells. In indicated cohorts, mice received dasatinib three days after T-cell transfer, thus on day 10, and then every 6 hours for a total of 8 doses. Based on the known pharmacokinetic and -dynamic of dasatinib in mice this provided a window between day 10 and day 12 after tumor inoculation where dasatinib was present in mouse serum at a concentration above the threshold required for blocking CAR-T cell function. As a control, a cohort of mice receiving CAR-T cells was additionally treated with dasatinib-free vehicle (indicated as CAR/DMSO).

CAR-T cell function is OFF in the presence of dasatinib, and re-ignites to function ON once dasatinib administration is discontinued.

The inventors analyzed the CAR-T cell antitumor function in mice that had received either CAR-T cells or untransduced control T cells, and had either received dasatinib according to the treatment schedule in FIG. 17A, or had not received dasatinib. Raji tumor burden was determined by bioluminescence imaging on day 7, day 10, day 12, day 14, day 17, and subsequently, once a week. The data show that in the first phase after T-cell transfer (day 7 to day 10), CD19-CAR T-cells commenced exerting their antilymphoma activity and were strongly activated, as demonstrated by decreasing BLI. At the same time, tumor grew rapidly in mice receiving CAR T cells and dasatinib (FIG. 17B). In the second phase after T-cell transfer (day 10 to day 12), dasatinib rapidly induced a function OFF state and halted antilymphoma reactivity, as tumor started to re-grow in 7 out of 10 animals in the dasatinib treated cohort. In contrast, the BLI signal was rapidly reduced during this phase in mice that had received CD19-CAR T-cells but no additional treatment in 9 out of 10 mice, and in 8 out of 10 mice that had received CD19-CAR T-cells and dasatinib-free vehicle. In the third phase (after day 12), administration of dasatinib was discontinued in order to allow CAR T-cells to revert back into their function ON state. Indeed, CAR T-cells rapidly resumed their antilymphoma function as revealed by rapidly decreasing BLI signal (FIG. 17B) that lead into even deeper remission on day 17 (median BLI of 507) when compared to mice receiving CAR-T cells and vehicle or CAR-T cells alone (median BLI of 966 and 839, respectively)

The data show that between day 7 and day 10, mice that received CAR-T cells showed a reduced tumor growth, indicating that T-cells have been activated (reduction of tumor by 75% (dasa), 15% (DMSO) and 11% (CAR only), respectively) when compared to mice receiving control T cells (growth rate of 1628%) (FIG. 17C). Between day 10 and day 12, mice that received CAR-T cells plus dasatinib showed tumor progression (CAR (ON/OFF/ON)); growth of 33%) as mice that had received CAR-T cells alone (reduction of BL by 32% (CAR/DMSO) and 61% (CAR/−)) (FIG. 17C), i.e. the CAR was ineffective in the presence of dasatinib despite primary activation of CAR-T cells.

The data show that between day 12 and 14, when dasatinib administration had been discontinued, there was a strong reduction in tumor burden in all groups that had been treated with CAR-T cells (9 out of 10 in CAR/DMSO, and 6/10 in CAR only cohorts), in particular there was a strong reduction in tumor burden in mice that had been previously treated with dasatinib (10 out of 10 mice, mean reduction of BLI by 71%), illustrating that the blockade of CAR-T function by dasatinib was rapidly reversible in vivo (FIG. 17C).

### Example 9: Dasatinib Exerts Superior Control Over CAR-T Cell Function Compared to Dexamethasone

The inventors prepared CD8⁺ CD19 CAR-T cell lines with a 4-1BB costimulatory domain from n=3 healthy donors. In each of the T-cell lines, the inventors enriched CAR expressing T cells to >90% purity using the EGFRt-transduction marker. The inventors performed functional testing using K562 that the inventors had transduced with CD19 as target cells to assess the influence of dexamethasone on CAR-T cell function. Dexamethasone was added to the assay medium either at the beginning of the assay, or used for 24-hour pretreatment in indicated dosages.

Dasatinib Exerts Superior Control Over the Cytolytic Function of CAR-T Cells Compared to Dexamethasone

The inventors analyzed cytolytic activity of CD8⁺ CAR-T cells in a bioluminescence-based cytotoxicity assay. The data show that dexamethasone is not capable of completely blocking the cytolytic function of CD8⁺ CAR-T cells expressing a CD19 CAR with 4-18B costimulatory domain. The extent of dexamethasone-induced inhibition of cytolytic function is not primarily dependent on dose, but rather depends on the treatment schedule:
When dexamethasone was added to the assay medium at the beginning of the assay, the cytolytic function of CAR-T cells was not significantly affected in any of the applied dosages (FIG. 18A, left panel) (>87% specific lysis of target cells by treated CAR-T cells compared to 91% specific lysis of target cells mediated by non-treated CAR-T cells).
When CAR-T cells were pre-treated for 24 h with dexamethasone (FIG. 18A, right panel), there was only partial inhibition of the cytolytic function of CAR-T cells at all tested doses (>45% specific lysis of target cells by dexamethasone-treated CAR-T cells at t=10 h compared to 91% specific lysis of target cells mediated by non-treated CAR-T cells).
Complete blockade of specific lysis of target cells mediated by CAR-T cells was observed for cells that had been treated with 0.1 μM dasatinib at the beginning of the assay, which was included into both panels as a reference and for comparison (<1% specific lysis at t=10 h).

Dasatinib Exerts Superior Control Over Cytokine Production and Secretion by CAR-T Cells Compared to Dexamethasone

The inventors analyzed the cytokine production and secretion by CD8⁺ CAR-T cell lines in the presence or absence of dexamethasone. ELISA was performed to detect IFN-γ and IL-2 in supernatant removed from the co-culture.

The data show that dexamethasone is not capable of completely blocking the cytokine secretion in CD8⁺ CAR-T cells expressing a CD19 CAR with 4-1BB costimulatory domain. The influence of dexamethasone on the secretion of cytokines depends on the treatment schedule and varies for different cytokines:
There was no significant reduction of IFN-γ secretion (FIG. 18B, left panel) for CAR-T cells that had been pre-treated (black bars) or had been treated during the assay only (grey bars). At any given concentration of dexamethasone, there was more than 43% of residual specific IFN-γ secretion by CAR-T cells that had been treated with dexamethasone compared to non-treated CAR-T cells.
There was a partial reduction of IL-2 secretion (FIG. 18B, right panel) for CAR-T cells that had been pre-treated (black bars) or had been treated with dexamethasone during the assay only (grey bars). At any given concentration of dexamethasone, there was less than 17% of residual specific IL-2 secretion by CAR-T cells that had been treated with dexamethasone compared to non-treated CAR-T cells. CAR-T cells that had been treated with 0.1 μM dasatinib showed a complete block of IL-2 secretion, consistent with the data obtained in Experiment 2, and were included as reference and for comparison.

Dasatinib Exerts Superior Control Over Proliferation of CAR-T Cells Compared to Dexamethasone

The inventors analyzed the proliferation of CD8⁺ CAR-T cell lines in the presence or absence of dexamethasone. CAR-T cells were labeled with CFSE and co-cultured with K562 that the inventors had transduced with CD19. Flow cytometry analyses were performed to determine the proliferation of T cells after 72 h. The proliferation index, indicating the average number of cell divisions performed during the assay period, was calculated, and was used to determine the remaining proliferation as normalized to the proliferation index of stimulated CAR-T cells in the absence of further treatment as 100%.

The data confirm that dexamethasone is able to reduce the proliferation of CD8⁺ CAR-T cells. The effects were equal between CAR-T cells that had received 24 h-pretreatment with dexamethasone and CAR-T cells that received dexamethasone at the start of co-culture. At any given concentration, the remaining proliferation was less than 26% compared to CAR-T cells that remained untreated (FIG. 18C). Nonetheless, a complete blockade of CAR-T cell proliferation as observed with 0.1 μM dasatinib (<5.6%), could not be accomplished by dexamethasone.

In aggregate, these data show that dasatinib exerts superior control over CAR-T cells compared to dexamethasone. In particular, the data show that administration of dexamethasone to CAR-T cells, neither at the start of the co-culture nor 24 h before the assay, can achieve a complete blockade of CAR-T cell functions as observed by treatment of CAR-T cells with 0.1 M dasatinib.

### Example 10: Tyrosine Kinase Inhibitors are Able to Influence CAR-T Cell Effector Functions

The Influence of Dasatinib and Other Clinically Approved Tyrosine Kinase Inhibitors on the Function of CAR-T Cells

The inventors prepared CD8⁺ ROR CAR-T cell lines with a 4-1BB costimulatory domain from n=2 healthy donors. In each of the T-cell lines, the inventors enriched CAR expressing T cells to >90% purity using the EGFRt-transduction marker. The inventors performed functional testing using ROR1⁺ RCH-ACV as target cells to assess the influence of a panel of clinically approved TKI on CAR-T cell function. TKIs were added to the assay medium at the beginning of the assay to a final concentration of ≥100 nM dasatinib, 5.3 μM imatinib, 4.2 μM lapatinib or 3.6 μM nilotinib.

Untreated CAR-T Cells were Used for Calculations and as a Control.

The inventors analyzed the cytolytic activity of CD8⁺ CAR-T cells in a bioluminescence-based cytotoxicity assay. The data show that of the tested panel, dasatinib is the only TKI that was capable of completely blocking the cytolytic function (specific lysis <5% at t=8 hours) (FIG. 19A). The data show that in the presence of lapatinib, nilotinib or imatinib, there was a partial inhibition of the cytolytic function of CAR-T cells (<75% specific lysis mediated by CAR-T cells treated with either lapatinib, nilotinib or imatinib compared to >90% specific lysis mediated by untreated CAR-T cells at t=8 hours).

The inventors analyzed the production and secretion of IFN-γ of the CD8⁺ CAR-T cells by performing ELISA using supernatant removed from the co-culture of CAR-T cells with RCH-ACV target cells. The data show that dasatinib and nilotinib are capable to reduce the amount of IFN-γ production and secretion (FIG. 19B):
In the presence of 100 nM dasatinib, the production and secretion of IFN-γ was completely blocked and below detection level.
In the presence of 3.6 M nilotinib, the production and secretion of IFN-γ was reduced to 480 pg/ml compared to 1310 pg/ml produced by untreated CAR-T cells, which resembles a remaining IFNγ secretion of 36.6%.

The inventors then analyzed the proliferation of CD8⁺ CAR-T cell lines in the presence or absence of TKI-treatment. CAR-T cells were labeled with CFSE and co-cultured with RCH-ACV. Proliferation of CAR-T cells was assessed by flow cytometry after 72 h of co-culture.

The data show that treatment with 100 nM dasatinib mediates a (near-)complete inhibition of CAR-T cell proliferation similar to the data shown in Example 2. The data also show that nilotinib is capable of partially blocking the proliferation of CD8⁺ CAR-T cells: at a concentration of 3.6 μM nilotinib in the assay medium, the proliferation index was reduced to 2.24 when compared to untreated CAR-T cells with a proliferation index of 2.84.

The Influence of Dasatinib and Other Src-Kinase Inhibitors on the Cytolytic Unction of CAR-T Cells

The inventors prepared CD8⁺ CD19 CAR-T cell lines with a 4-1BB costimulatory domain from one healthy donor. In the T-cell line, the inventors enriched CAR expressing T cells to >90% purity using the EGFRt-transduction marker. The inventors analyzed cytolytic activity of CD8⁺ CAR-T cells in a 4-hour bioluminescence-based cytotoxicity assay using K562 that the inventors had transduced with CD19 as target cells to assess the influence of a panel of Src-kinase inhibitors on the cytolytic function of CAR-T cells. Src-kinase inhibitors were added to the assay medium at the beginning of the assay over a 4-log concentration range.

The data show that of the four tested Src kinase inhibitors, three inhibitors are capable of blocking the cytolytic activity of CAR-T cells (FIG. 20):
at a concentration of ≥10 nM of dasatinib in the assay medium, there was a partial inhibition of cytolytic function of CAR-T cells (16.1% specific lysis of target cells compared to 82% specific lysis of target cells by untreated CAR-T cells).
at a concentration of ≥100 nM of dasatinib in the assay medium, there was a (near-) complete inhibition of cytolytic function of CAR-T cells (<3% specific lysis of target cells compared to 82% specific lysis of target cells by untreated CAR-T cells).
at a concentration of ≥10 nM of PP1-inhibitor in the assay medium, there was a partial inhibition of cytolytic function of CAR-T cells (62.4% specific lysis of target cells compared to 82% specific lysis of target cells by untreated CAR-T cells).
at a concentration of ≥100 nM of PP1-inhibitor in the assay medium, there was a (near-) complete inhibition of cytolytic function of CAR-T cells (<3% specific lysis of target cells compared to 82% specific lysis of target cells by untreated CAR-T cells).
at a concentration of ≥1000 nM of bosutinib in the assay medium, there was a (near-) complete inhibition of cytolytic function of CAR-T cells (<3% specific lysis of target cells compared to 82% specific lysis of target cells by untreated CAR-T cells).

In aggregate, these data show that tyrosine kinase inhibitors other than dasatinib can exert an inhibitory effect to CAR-T cell functions. In particular, the data show that nilotinib is a potent inhibitor for cytokine production and secretion from CAR-T cells. The Src-kinase inhibitors PP1-inhibitor and bosutinib are able to completely block the cytolytic function of CD8⁺ CAR-T cells.

### Example 11: Intermittent Treatment with Dasatinib Augments CAR-T Cell Function

Intermittent Exposure to Dasatinib Augments the Antitumor Function of CAR-T Cells In Vivo

The inventors employed a xenograft model in immunodeficient mice (NSG/Raji) to assess the influence of dasatinib on CD19 CAR/4-1BB-T cells in vivo. The experiment setup and treatment schedule is provided in FIG. 21A. In brief, cohorts of n2 mice were inoculated with 1×10\hat{ }6 firefly-luciferase_GFP-transduced Raji tumor cells on day 0. CAR-T cells (i.e. CD8⁺ and CD4⁺ T cells expressing a CD19 CAR with 4-1BB costimulatory domain, total dose: 5×10e6; CD8:CD4 ratio=1:1) or control untransduced T cells were administered on day 7 by i.v. tail vein injection. 5 mg/kg Dasatinib was administered by i.p. injection every 24 hours from d7 until d11 followed by i.p. injection every 36 hours on d12 and 14 (total 7 doses). Serial bioluminescence imaging was performed to determine tumor burden on day 7, 9 and 15.

Based on the known pharmacokinetic and—dynamic of dasatinib in mice [23], this provided a window of ^{˜}6 hours after each injection when dasatinib was present in mouse serum at a concentration of >50 nM, which should lead to a temporary blockade of CAR-T cell function as shown in Example 2. For the following 21 hours (until the next injection), dasatinib should be below the inhibitory threshold of 50 nM and therefore should not have inhibitory effects on CAR-T cell function.

The data show that intermittent treatment of mice with dasatinib increases the antitumor function of CAR-T cells in vivo (FIG. 21B). Mice that had received CAR-T cells and dasatinib showed superior tumor control and slower tumor progression compared to mice that had received CAR-T cells without dasatinib: On day 8, the average bioluminescence signal in mice that had received CAR-T cells without dasatinib was 1.9e10 p/s/cm*2/sr, whereas in mice that received CAR-T cells and intermittent treatment with dasatinib the average bioluminescence signal was only 5.6e9 p/s/cm*2/sr (p<0.05). On day 8, there was no statistically significant difference in bioluminescence signal between mice that had received untransduced control T cells with or without dasatinib.

Intermittent Exposure to Dasatinib Augments the Engraftment, Proliferation and Persistence of CAR-T Cells In Vivo

The inventors used a xenograft model as described in FIG. 21A to analyze CAR-T cell engraftment, proliferation and persistence in mice with intermittent exposure to dasatinib. On day 15, mice were sacrificed and peripheral blood (PB), bone marrow (BM) and spleen (SP) analyzed for the presence of human CAR-T cells by flow cytometry. The gating strategy used to assess the percentage of live human T cells (7AAD⁻, CD3⁺, CD45⁺) and of remaining tumor cells (GFP⁺) is displayed in FIG. 22A.

The data show that intermittent exposure to dasatinib augments the antitumor function of CAR-T cells, as has been shown in FIG. 21B. A high tumor burden of 58.8% GFP positive tumor cells of all living cells was detected in the bone marrow of one individual mouse that had been treated with CAR-T cells (FIG. 22A, upper panel). In contrast to that, one exemplary mouse treated with CAR-T cells and intermittent dasatinib showed a remaining tumor burden of 0.22% of all living cells in the bone marrow (FIG. 22A, lower panel).

The data in FIG. 22B show that intermittent treatment with dasatinib augments the engraftment, proliferation and persistence of CAR-T cells in vivo. In bone marrow and spleen, the percentage of human CAR-T cells was higher in animals that had been treated with intermittent dasatinib (BM: 7.3%; SP: 6.9%) when compared to animals that had received CAR-T cells but no intermittent dasatinib (BM: 1.9%, SP: 3.2%) (p>0.05).

In aggregate, these data show that intermittent exposure to dasatinib augments the antitumor function of CAR-T cells in vivo; intermittent exposure to dasatinib also augments the engraftment, proliferation and persistence of CAR-T cells in vivo.

### Example 12: Intermittent Treatment with Dasatinib Decreases PD-1 Expression on CAR-T Cells

Based on the mouse model introduced in Example 11 (FIG. 21A), the inventors analyzed the surface expression of PD-1 on human CAR-T cells in bone marrow (BM), peripheral blood (PB) and spleen (SP) by flow cytometry.

The data show that intermittent exposure of dasatinib significantly reduces PD-1 expression in CAR-T cells in bone marrow and peripheral blood compared to CAR-T cells in corresponding organs of mice that were not exposed to intermittent dasatinib (FIG. 23):
In bone marrow (BM), the mean fluorescence intensity (MFI) obtained after staining CAR-T cells with an anti-PD1 mAb was 9461 in mice that had not been exposed to dasatinib (CAR/−), and was only 7025 in mice that had been intermittently treated with dasatinib (CAR/+).
In peripheral blood (PB), the mean fluorescence intensity (MFI) obtained after staining CAR-T cells with an anti-PD1 mAb was 4110 in mice that had not been exposed to dasatinib (CAR/−), and was only 2775 in mice that had been intermittently treated with dasatinib (CAR/+).
In spleen (SP), the mean fluorescence intensity (MFI) obtained after staining CAR-T cells with an anti-PD1 mAb was 4318 in mice that had not been exposed to dasatinib (CAR/−), and was only 23652 in mice that had been intermittently treated with dasatinib (CAR/+).

In aggregate, these data show that by intermittent exposure, dasatinib decreases expression of PD-1 on CAR-T cells.

### Example 13: CAR-T Cells that are Blocked by Dasatinib are Susceptible to Subsequent Elimination with the iCasp9 Suicide Gene

CAR-T cells co-expressing the iCasp suicide gene were cultured in medium supplemented with 50 U/ml IL-2, either in the absence or in the presence of 100 nM dasatinib, and in the absence or presence of 10 nM AP20187, which is an iCaspase inducer drug. After 24 hours, cells were labeled with anti-CD3 mAB and analyzed by flow cytometry for the presence of iCasp+ T cells.

The data show that the induction suicide genes and following apoptosis of T cells is not affected by dasatinib (FIG. 24):

In the presence of dimerizer (dasatinib/dimerizer +), the percentage of iCasp⁺ cells was reduced to 45%, which was comparable to the percentage of iCasp⁺ in the presence of 100 nM dasatinib (36%) and dimerizer (dasatinib +/dimerizer +).

In aggregate, these data show that CAR-T cells that are blocked by dasatinib are susceptible to subsequent elimination with the iCasp9 suicide gene.

### INDUSTRIAL APPLICABILITY

The immune cells and tyrosine kinase inhibitors for the uses according to the invention, as well as materials used for the methods of the invention, can be industrially manufactured and sold as products for the claimed methods and uses (e.g. for treating a cancer as defined herein), in accordance with known standards for the manufacture of pharmaceutical and diagnostic products. Accordingly, the present invention is industrially applicable.

### REFERENCES

[1] J. N. Kochenderfer et al, “Lymphoma remissions caused by anti-CD19 chimeric antigen receptor T cells are associated with high serum interleukin-15 levels,” J. Clin. Oncol., 2017.
[2] C. J. Turtle et al., “Immunotherapy of non-Hodgkins lymphoma with a defined ratio of CD8⁺ and CD4⁺ CD19-specific chimeric antigen receptor-modified T cells,” Sci. Transl. Med., 2016.
[3] C. J. Turtle et al., “CD19 CAR-T cells of defined CD4⁺:CD8⁺ composition in adult B cell ALL patients,” J. Clin. Invest., 2016.
[4] S. A. Ali et al., “T cells expressing an anti-B-cell maturation antigen chimeric antigen receptor cause remissions of multiple myeloma.,” Blood, vol. 128, no. 13, pp. 1688-700, September 2016.
[5] M. L. Davila et al., “Efficacy and Toxicity Management of 19-28z CAR T Cell Therapy in B Cell Acute Lymphoblastic Leukemia,” Sci. Transl. Med., 2014.
[6] D. W. Lee et al., “Current concepts in the diagnosis and management of cytokine release syndrome,” Blood, 2014.
[7] R. J. Brentjens et al., “CD19-Targeted T Cells Rapidly Induce Molecular Remissions in Adults with Chemotherapy-Refractory Acute Lymphoblastic Leukemia,” Sci. Transl. Med., p. sc, 2013.
[8] I. Diaconu et al., “Inducible Caspase-9 Selectively Modulates the Toxicities of CD19-Specific Chimeric Antigen Receptor-Modified T Cells,” Mol. Ther., vol. 25, no. 3, pp. 580-592, March 2017.
[9] X. Wang et al., “A transgene-encoded cell surface polypeptide for selection, in vivo tracking, and ablation of engineered cells,” Blood, 2011.
[10] J. Gust et al., “Endothelial Activation and Blood-Brain Barrier Disruption in Neurotoxicity after Adoptive Immunotherapy with CD19 CAR-T Cells,” Cancer Discov., 2017.
[11] J. Weber, “Immune checkpoint proteins: A new therapeutic paradigm for cancerpreclinical background: CTLA-4 and PD-1 blockade,” Seminars in Oncology. 2010.
[12] J. S. Tokarski et al., “The structure of dasatinib (BMS-354825) bound to activated ABL kinase domain elucidates its inhibitory activity against imatinib-resistant ABL mutants,” Cancer Res., vol. 66, no. 11, pp. 5790-5797, June 2006.
[13] P. C. Nowell and D. A. Hungerford, “Chromosome Studies on Normal and Leukemic Human Leukocytes 1.”
[14] D. Catovsky et al., “Multiparameter studies in lymphoid leukemias.,” Am. J. Clin. Pathol., vol. 72, no. 4 Suppl, pp. 736-45, October 1979.
[15] S. Blake, T. P. Hughes, G. Mayrhofer, and A. B. Lyons, “The Src/ABL kinase inhibitor dasatinib (BMS-354825) inhibits function of normal human T-lymphocytes in vitro,” Clin. Immunol., vol. 127, no. 3, pp. 330-339, June 2008.
[16] F. Fei et al., “Dasatinib exerts an immunosuppressive effect on CD8+ T cells specific for viral and leukemia antigens,” Exp. Hematol., vol. 36, no. 10, pp. 1297-1308, October 2008.
[17] C. K. Fraser et al., “Dasatinib inhibits recombinant viral antigen-specific murine CD4+ and CD8+ T-cell responses and NK-cell cytolytic activity in vitro and in vivo,” Exp. Hematol., vol. 37, no. 2, pp. 256-265, February 2009.
[18] R. Weichsel et al., “Profound Inhibition of Antigen-Specific T-Cell Effector Functions by Dasatinib,” Clin. Cancer Res., vol. 14, no. 8, pp. 2484-2491, March 2008.
[19] M. Kalos et al., “T Cells with Chimeric Antigen Receptors Have Potent Antitumor Effects and Can Establish Memory in Patients with Advanced Leukemia,” Sci. Transl. Med., 2011.
[20] J. N. Kochenderfer et al., “Donor-derived CD19-targeted T cells cause regression of malignancy persisting after allogeneic hematopoietic stem cell transplantation,” Blood, vol. 122, no. 25, pp. 4129-4139, December 2013.
[21] K. C. Straathof et al., “An inducible caspase 9 safety switch for T-cell therapy,” Blood, 2005.
[22] M. Hudecek et al., “Receptor affinity and extracellular domain modifications affect tumor recognition by ROR1-specific chimeric antigen receptor T cells,” Clin. Cancer Res., 2013.
[23] F. R. Luo et al., “Dasatinib (BMS-354825) Pharmacokinetics and Pharmacodynamic Biomarkers in Animal Models Predict Optimal Clinical Exposure,” Clin. Cancer Res., vol. 12, no. 23, pp. 7180-7186, December 2006.

Sequences

The following amino acid sequences are part of the construct “CD19 CAR with 4-1BB
costimulatory domain” (see FIG. 1A):
SEQ ID NO: 1 (GMCSF signal peptide):
MLLLVTSLLLCELPHPAFLLIP
SEQ ID NO: 2 (CD19 heavy chain variable domain (VH));
DIQMTQTTSSLSASLGDRVTISCRASQDISKYLNWYQQKPDGTVKLLIYHTSRLHSGVPSRFSGSGSGTDYSLTI
SNLEQEDIATYFCQQGNTLPYTFGGGTKLEITGSTSGSGKPGSGEGSTKGEVKLQESGPGLVAPSQSLSVTCTV
SGVSLPDYGVSWIRQPPRKGLEWLGVIWGSETTYYNSALKSRLTIIKDNSKSQVFLKMNSLQTDDTAIYYCAK
HYYYGGSYAMDYWGQGTSVTVSS
SEQ ID NO: 3 (IgG4 hinge domain):
ESKYGPPCPPCP
SEQ ID NO: 4 (CD28 transmembrane domain):
MFWVLVVVGGVLACYSLLVTVAFIIFWV
SEQ ID NO: 5 (4-1BB costimulatory domain):
KRGRKKLLYIFKQPFMRPVQTTQEEDGCSCRFPEEEEGGCEL
SEQ ID NO: 6 (CD3z signaling domain):
RVKFSRSADAPAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
SEQ ID NO: 7 (T2A ribosomal skipping sequence):
LEGGGEGRGSLLTCGDVEENPGPR
SEQ ID NO: 8 (GMCSF signal peptide):
MLLLVTSLLLCELPHPAFLLIP
SEQ ID NO: 9 (EGFRt):
RKVCNGIGIGEFKDSLSINATNIKHFKNCTSISGDLHILPVAFRGDSFTHTPPLDPQELDILKTVKEITGFLLIQAW
PENRTDLHAFENLEIIRGRTKQHGQFSLAVVSLNITSLGLRSLKEISDGDVIISGNKNLCYANTINWKKLFGTSG
QKTKIISNRGENSCKATGQVCHALCSPEGCWGPEPRDCVSCRNVSRGRECVDKCNLLEGEPREFVENSECIQ
CHPECLPQAMNITCTGRGPDNCIQCAHYIDGPHCVKTCPAGVMGENNTLVWKYADAGHVCHLCHPNCTY
GCTGPGLEGCPTNGPKIPSIATGMVGALLLLLVVALGIGLFM
The following amino acid sequences are part of the construct “CD19 CAR with CD28
costimulatory domain” (see FIG. 18):
SEQ ID NO: 10 (GMCSF signal peptide):
MLLLVTSLLLCELPHPAFLLIP
SEQ ID NO: 11 (CD19 scFv):
DIQMTQTTSSLSASLGDRVTISCRASQDISKYLNWYQQKPDGTVKLLIYHTSRLHSGVPSRFSGSGSGTDYSLTI
SNLEQEDIATYFCQQGNTLPYTFGGGTKLEITGSTSGSGKPGSGEGSTKGEVKLQESGPGLVAPSQSLSVTCTV
SGVSLPDYGVSWIRQPPRKGLEWLGVIWGSETTYYNSALKSRLTIIKDNSKSQVFLKMNSLQTDDTAIYYCAK
HYYYGGSYAMDYWGQGTSVTV
SEQ ID NO: 12 (IgG4 hinge domain):
ESKYGPPCPPCP
SEQ ID NO: 13 (CD28 transmembrane domain):
MFWVLVVVGGVLACYSLLVTVAFIIFWV
SEQ ID NO: 14 (CD28 costimulatory domain):
RSKRSRGGHSDYMNMTPRRPGPTRKHYQPYAPPRDFAAYRS
SEQ ID NO: 15 (CD3z signaling domain):
RVKFSRSADAPAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
SEQ ID NO: 16 (T2A ribosomal skipping sequence):
LEGGGEGRGSLLTCGDVEENPGPR
SEQ ID NO: 17 (GMCSF signal peptide):
MLLLVTSLLLCELPHPAFLLIP
SEQ ID NO: 18 (EGFRt):
RKVCNGIGIGEFKDSLSINATNIKHFKNCTSISGDLHILPVAFRGDSFTHTPPLDPQELDILKTVKEITGFLLIQAW
PENRTDLHAFENLEIIRGRTKQHGQFSLAVVSLNITSLGLRSLKEISDGDVIISGNKNLCYANTINWKKLFGTSG
QKTKIISNRGENSCKATGQVCHALCSPEGCWGPEPRDCVSCRNVSRGRECVDKCNLLEGEPREFVENSECIQ
CHPECLPQAMNITCTGRGPDNCIQCAHYIDGPHCVKTCPAGVMGENNTLVWKYADAGHVCHLCHPNCTY
GCTGPGLEGCPTNGPKIPSIATGMVGALLLLLVVALGIGLFM
The following amino acid sequences are part of the construct “ROR1 CAR with 4-1BB
costimulatory domain” (see FIG. 1C):
SEQ ID NO: 19 (GMCSF signal peptide):
MLLLVTSLLLCELPHPAFLLIP
SEQ ID NO: 20 (hR12 heavy chain variable domain (VH)):
QVQLVESGGALVQPGGSLTLSCKASGFDFSAYYMSWVRQAPGKGLEWIATIYPSSGKTYYAASVQGRFTISA
DNAKNTVYLQMNSLTAADTATYFCARDSYADDGALFNIWGQGTLVTVSS
SEQ ID NO: 21 (4(GS)x3 linker):
GGGGSGGGGSGGGGS
SEQ ID NO: 22 (hR12 light chain variable domain (VL)):
QLVLTQSPSVSAALGSSAKITCTLSSAHKTDTIDWYQQLAGQAPRYLMYVQSDGSYEKRSGVPDRFSGSSSG
ADRYLIISSVQADDEADYYCGADYIGGYVFGGGTQLTVG
SEQ ID NO: 23 (IgG4 hinge domain):
ESKYGPPCPPCP
SEQ ID NO: 24 (CO28 transmembrane domain):
MFWVLVVVGGVLACYSLLVTVAFIIFWV
SEQ ID NO: 25 (4-1BB costimulatory domain):
KRGRKKLLYIFKQPFMRPVQTTQEEDGCSCRFPEEEEGGCEL
SEQ ID NO: 26 (CD3z signaling domain):
RVKFSRSADAPAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
SEQ ID NO: 27 (T2A ribosomal skipping sequence):
LEGGGEGRGSLLTCGDVEENPGPR
SEQ ID NO: 28 (GMCSF signal peptide):
MLLLVTSLLLCELPHPAFLLIP
SEQ ID NO: 29 (EGFRt):
RKVCNGIGIGEFKDSLSINATNIKHFKNCTSISGDLHILPVAFRGDSFTHTPPLDPQELDILKTVKEITGFLLIQAW
PENRTDLHAFENLEIIRGRTKQHGQFSLAVVSLNITSLGLRSLKEISDGDVIISGNKNLCYANTINWKKLFGTSG
QKTKIISNRGENSCKATGQVCHALCSPEGCWGPEPRDCVSCRNVSRGRECVDKCNLLEGEPREFVENSECIQ
CHPECLPQAMNITCTGRGPDNCIQCAHYIDGPHCVKTCPAGVMGENNTLVWKYADAGHVCHLCHPNCTY
GCTGPGLEGCPTNGPKIPSIATGMVGALLLLLVVALGIGLFM
The following amino acid sequences are part of the construct “SLAMF7 CAR with 4-1BB
costimulatory domain” (see FIG. 1D):
SEQ ID NO: 30 (GMCSF signal peptide):
MLLLVTSLLLCELPHPAFLLIP
SEQ ID NO: 31 (huLuc63 heavy chain variable domain (VH)):
EVQLVESGGGLVQPGGSLRLSCAASGFDFSRYWMSWVRQAPGKGLEWIGEINPDSSTINYAPSLKDKFIISR
DNAKNSLYLQMNSLRAEDTAVYYCARPDGNYWYFDVWGQGTLVTVSS
SEQ ID NO: 32 (4(GS)x3 linker):
GGGGSGGGGSGGGGS
SEQ ID NO: 33 (huLuc63 light chain variable domain (VL)):
DIQMTQSPSSLSASVGDRVTITCKASQDVGIAVAWYQQKPGKVPKLLIYWASTRHTGVPDRFSGSGSGTDFT
LTISSLQPEDVATYYCQQYSSYPYTFGQGTKVEIK
SEQ ID NO: 34 (IgG4 hinge domain):
ESKYGPPCPPCPAPPVAGPSVFLFPPKPKDTLMISRTPEVTCVVVDVSQEDPEVQFNWYVDGVEVHNAKTK
PREEQFQSTYRVVSVLTVLHQDWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNVFSCSVMHEALHN
HYTQKSLSLSLGK
SEQ ID NO: 35 (CD28 transmembrane domain):
MFWVLVVVGGVLACYSLLVTVAFIIFWV
SEQ ID NO: 36 (4-1BB costimulatory domain):
KRGRKKLLYIFKQPFMRPVQTTQEEDGCSCRFPEEEEGGCEL
SEQ ID NO: 37 (CD3z signaling domain):
RVKFSRSADAPAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
SEQ ID NO: 38 (T2A ribosomal skipping sequence):
LEGGGEGRGSLLTCGDVEENPGPR
SEQ ID NO: 39 (GMCSF signal peptide):
MLLLVTSLLLCELPHPAFLLIP
SEQ ID NO: 40 (EGFRt):
RKVCNGIGIGEFKDSLSINATNIKHFKNCTSISGDLHILPVAFRGDSFTHTPPLDPQELDILKTVKEITGFLLIQAW
PENRTDLHAFENLEIIRGRTKQHGQFSLAVVSLNITSLGLRSLKEISDGDVIISGNKNLCYANTINWKKLFGTSG
QKTKIISNRGENSCKATGQVCHALCSPEGCWGPEPRDCVSCRNVSRGRECVDKCNLLEGEPREFVENSECIQ
CHPECLPQAMNITCTGRGPDNCIQCAHYIDGPHCVKTCPAGVMGENNTLVWKYADAGHVCHLCHPNCTY
GCTGPGLEGCPTNGPKIPSIATGMVGALLLLLVVALGIGLFM
The following amino acid sequences are part of the construct “SLAMF7 CAR with CD28
costimulatory domain” (see FIG. 1E):
SEQ ID NO: 41 (GMCSF signal peptide):
MLLLVTSLLLCELPHPAFLLIP
SEQ ID NO: 42 (huLuc63 heavy chain variable domain (VH)):
EVQLVESGGGLVQPGGSLRLSCAASGFDFSRYWMSWVRQAPGKGLEWIGEINPDSSTINYAPSLKDKFIISR
DNAKNSLYLQMNSLRAEDTAVYYCARPDGNYWYFDVWGQGTLVTVSS
SEQ ID NO: 43 (4(GS)x3 linker):
GGGGSGGGGSGGGGS
SEQ ID NO: 44 (huLuc63 light chain variable domain (VL)):
DIQMTQSPSSLSASVGDRVTITCKASQDVGIAVAWYQQKPGKVPKLLIYWASTRHTGVPDRFSGSGSGTDFT
LTISSLQPEDVATYYCQQYSSYPYTFGQGTKVEIK
SEQ ID NO: 45 (IgG4 hinge domain):
ESKYGPPCPPCPAPPVAGPSVFLFPPKPKDTLMISRTPEVTCVVVDVSQEDPEVQFNWYVDGVEVHNAKTK
PREEQFQSTYRVVSVLTVLHQDWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNVFSCSVMHEALHN
HYTQKSLSLSLGK
SEQ ID NO: 46 (CD28 transmembrane domain):
MFWVLVVVGGVLACYSLLVTVAFIIFWV
SEQ ID NO: 47 (CD28 costimulatory domain):
RSKRSRGGHSDYMNMTPRRPGPTRKHYQPYAPPRDFAAYRS
SEQ ID NO: 48 (CD3z signaling domain):
RVKFSRSADAPAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
SEQ ID NO: 49 (T2A ribosomal skipping sequence):
LEGGGEGRGSLLTCGDVEENPGPR
SEQ ID NO: 50 (GMCSF signal peptide):
MLLLVTSLLLCELPHPAFLLIP
SEQ ID NO: 51 (EGFRt):
RKVCNGIGIGEFKDSLSINATNIKHFKNCTSISGDLHILPVAFRGDSFTHTPPLDPQELDILKTVKEITGFLLIQAW
PENRTDLHAFENLEIIRGRTKQHGQFSLAVVSLNITSLGLRSLKEISDGDVIISGNKNLCYANTINWKKLFGTSG
QKTKIISNRGENSCKATGQVCHALCSPEGCWGPEPRDCVSCRNVSRGRECVDKCNLLEGEPREFVENSECIQ
CHPECLPQAMNITCTGRGPDNCIQCAHYIDGPHCVKTCPAGVMGENNTLVWKYADAGHVCHLCHPNCTY
GCTGPGLEGCPTNGPKIPSIATGMVGALLLLLVVALGIGLFM

1-148. (canceled)

149. A method of treatment comprising:
administering a composition comprising a tyrosine kinase inhibitor to a cancer patient that is undergoing immunotherapy.

150. The method of claim 149, wherein the immunotherapy is adoptive immunotherapy and/or immunotherapy with immune cells, optionally with immune cells expressing a chimeric antigen receptor, optionally wherein said chimeric antigen receptor is capable of binding to an antigen, optionally to a cell surface antigen, further optionally wherein said antigen is a cancer antigen, and/or wherein said antigen is selected from the group consisting of CD4, CD5, CD10, CD19, CD20, CD22, CD27, CD30, CD33, CD38, CD44v6, CD52, CD64, CD70, CD72, CD123, CD135, CD138, CD220, CD269, CD319, ROR1, ROR2, SLAMF7, BCMA, αvβ3-Integrin, α4β1-Integrin, LILRB4, EpCAM-1, MUC-1, MUC-16, L1-CAM, c-kit, NKG2D, NKG2D-Ligand, PD-L1, PD-L2, Lewis-Y, CAIX, CEA, c-MET, EGFR, EGFRvIII, ErbB2, Her2, FAP, FR-a, EphA2, GD2, GD3, GPC3, IL-13Ra, Mesothelin, PSMA, PSCA, VEGFR, and FLT3, and/or wherein said cancer comprises cancer cells which express said antigen, further optionally, said chimeric antigen receptor comprises a costimulatory domain selected from the group consisting of the CD27, CD28, 4-1BB, ICOS, DAP10, NKG2D, MyD88 and OX40 costimulatory domains, and, further optionally, said immune cells are lymphocytes, optionally B lymphocytes or T lymphocytes, optionally CD4⁺ and/or CD8⁺ T lymphocytes, and/or wherein said immune cells are selected from the group consisting of CD8⁺ killer T cells, CD4⁺ helper T cells, naïve T cells, memory T cells, central memory T cells, effector memory T cells, memory stem T cells, invariant T cells, NKT cells, cytokine induced killer T cells, gamma/delta T cells, natural killer cells, monocytes, macrophages, dendritic cells, and granulocytes.

151. The method of claim 149, wherein said tyrosine kinase inhibitor is a Src kinase inhibitor, and/or wherein said tyrosine kinase inhibitor is an inhibitor of kinases upstream of NFAT and/or an Lck kinase inhibitor, and/or wherein said tyrosine kinase inhibitor is selected from the group consisting of dasatinib, saracatinib, bosutinib, nilotinib, and PP1-inhibitor.

152. The method of claim 150, wherein said tyrosine kinase inhibitor causes inhibition of said immune cells, optionally wherein said inhibition is an inhibition of cell mediated effector functions of said immune cells, and/or wherein said inhibition of said immune cells is an inhibition of their
I) cytolytic activity; and/or
II) cytokine secretion; and/or
III) proliferation;
and/or
wherein said inhibition comprises inhibition of PD1 expression in said immune cells, and/or wherein said inhibition comprises inhibition of cytokine secretion of said immune cells of one or more cytokines selected from the group consisting of GM-CSF, IFN-γ, IL-2, IL-4, IL-5, IL-6, IL-8, and IL-10, and/or wherein said inhibition is a partial inhibition or a complete inhibition, and/or wherein said inhibition does not decrease the viability of said immune cells, optionally does not decrease the viability of said immune cells for a given time period during which said composition is administered to said patient, wherein said time period is 1 hour, preferably 2 hours, preferably 3 hours, preferably 4 hours, preferably 5 hours, preferably 6 hours, preferably 8 hours, preferably 12 hours, preferably 18 hours, preferably 1 day, preferably 2 days, more preferably 3 days, even more preferably 7 days, even more preferably 2 weeks, even more preferably 3 weeks, even more preferably 4 weeks, even more preferably 2 months, even more preferably 3 months, even more preferably 6 months, and/or wherein said inhibition is reversible, optionally wherein said reversible inhibition is reversed after said composition has not been administered to said patient for a given amount of time, optionally wherein said given amount of time is 3 days, preferably 2 days, more preferably 24 hours, even more preferably 18 hours, even more preferably 12 hours, even more preferably 8 hours, even more preferably 6 hours, even more preferably 4 hours, even more preferably 3 hours, even more preferably 2 hours, even more preferably 90 minutes, even more preferably 60 minutes, even more preferably 30 minutes.

153. The method of claim 149, wherein said composition is to be administered continuously or intermittently, and/or wherein the composition is to be administered such that after initial administration of said composition the serum levels of said tyrosine kinase inhibitor are maintained at or above a threshold serum level during the duration of said treatment, and/or wherein in the method, the composition is to be administered such that after initial administration of said composition the serum levels of said tyrosine kinase inhibitor are maintained at least once above a threshold serum level and a least once below the same threshold serum level during the duration of said treatment, optionally wherein said threshold serum level is within the range of 0.1 nM-1 μM, preferably 1 nM-500 nM, more preferably 5 nM-100 nM, even more preferably 10 nM-75 nM, even more preferably 25 nM-50 nM, further optionally wherein said threshold serum level is 50 nM, and/or wherein said threshold serum level is the minimum serum level at which said inhibition of said immune cells is a complete inhibition of their
I) cytolytic activity; and/or
II) cytokine secretion; and/or
III) proliferation.

154. The method of claim 149, wherein:
a) said treatment of cancer has an improved clinical outcome compared to said immunotherapy against said cancer alone;
b) said use is a use for mitigating or preventing toxicity associated with said immunotherapy against said cancer;
c) said use is a use for decreasing tumor burden in said patient compared to said immunotherapy against said cancer alone;
d) said use in the treatment of cancer does not decrease the therapeutic efficacy of said immunotherapy against said cancer compared to said immunotherapy against said cancer alone;
e) said use in the treatment of cancer is a use for increasing the therapeutic efficacy of said immunotherapy against said cancer compared to said immunotherapy against said cancer alone;
f) said use in the treatment of cancer is a use for decreasing the morbidity and mortality of said immunotherapy against said cancer compared to said immunotherapy against said cancer alone; and/or
g) said use in the treatment of cancer is a use for increasing the anti-tumor efficacy of said immunotherapy against said cancer compared to said immunotherapy against said cancer alone.

155. The method of claim 150, wherein said immunotherapy is immunotherapy with immune cells, wherein said use in the treatment of cancer is a use for increasing the engraftment and/or persistence of said immune cells in said immunotherapy against said cancer compared to the engraftment and/or persistence of said immune cells in said immunotherapy against said cancer alone, and/or wherein said use in the treatment of cancer is a use for increasing the engraftment of said immune cells in said immunotherapy compared to the engraftment of said immune cells in a method comprising said immunotherapy against said cancer alone, and/or wherein said use is a use for decreasing the exhaustion of said immune cells in said immunotherapy against said cancer compared to the exhaustion of said immune cells in a method comprising said immunotherapy against said cancer alone.

156. The method of claim 149, wherein said composition is to be administered
I) before said treatment of cancer by immunotherapy; and/or
II) concurrently to said treatment of cancer by immunotherapy; and/or
III) after said treatment of cancer by immunotherapy,
and/or wherein said immunotherapy is immunotherapy with immune cells and wherein said use is a use for preventing activation of said immune cells in said immunotherapy, optionally wherein said immune cells are resting immune cells, and/or wherein said immunotherapy is immunotherapy with immune cells and wherein said immune cells are of human origin, optionally wherein said immune cells of human origin are primary human cells, optionally wherein said primary human cells are primary human T lymphocytes, and/or wherein said immune cells of human origin are allogeneic or syngeneic cells with respect to said patient, and/or wherein said immune cells are immune cells which transiently or stably express said chimeric antigen receptor, and/or wherein said chimeric antigen receptor is of first, second, or third generation, and/or wherein said chimeric antigen receptor is capable of binding to an antigen and wherein said chimeric antigen receptor comprises a single chain variable fragment, preferably wherein said single chain variable fragment is capable of binding to said antigen, and/or wherein said chimeric antigen receptor is capable of binding to an antigen and wherein said chimeric antigen receptor comprises a ligand or fragment thereof, wherein said ligand or fragment thereof is capable of binding to said antigen, and/or wherein said chimeric antigen receptor is capable of binding to an antigen and wherein said chimeric antigen receptor comprises a signaling domain comprising one or more domains selected from the group consisting of CD3 zeta, CD3 epsilon, CD3 gamma, T-cell receptor alpha chain, T-cell receptor beta chain, T-cell receptor delta chain, and T-cell receptor gamma chain.

157. The method of claim 149, wherein said cancer is a cancer associated with a higher risk of morbidity and mortality in said immunotherapy, optionally wherein said cancer comprises cells which express one or more checkpoint molecules, which are preferably selected from the group consisting A2AR, B7-H3, B7-H4, BTLA, CTLA-4, IDO, KIR, LAG3, PD-L1, PD-L2, TIM-3, and VISTA, and/or wherein said cancer is a cancer selected from the group consisting of carcinoma, sarcoma, myeloma, leukemia, and lymphoma, optionally wherein said carcinoma selected from the group consisting of breast cancer, lung cancer, colorectal cancer, and pancreatic cancer, or optionally wherein said leukemia is B-cell leukemia, T-cell leukemia, myeloid leukemia, acute lymphoblastic leukemia, or chronic myeloid leukemia, or optionally wherein said lymphoma is non-Hodgkin lymphoma, Hodgkin lymphoma, or B-cell lymphoma, and/or wherein said cancer is a cancer characterized as
I) CD19 positive; and/or
II) BCMA positive; and/or
III) ROR1 positive; and/or
IV) FLT3 positive; and/or
V) CD20 positive; and/or
VI) CD22 positive; and/or
VII) CD123 positive; and/or
VIII) SLAMF7 positive;
and/or wherein said patient is a patient that is not eligible for said treatment of said cancer by said immunotherapy alone, and/or wherein said patient is a patient that is not eligible for conventional adoptive immunotherapy with T cells expressing a chimeric antigen receptor, and/or wherein said patient has an increased risk of developing cytokine release syndrome, and/or wherein said patient has an increased risk of developing neurotoxic side effects associated with said immunotherapy, and/or wherein said patient has an increased risk of developing on-target/off-tumor effects associated with said immunotherapy, and/or wherein said patient has elevated serum levels of one or more cytokines selected from the group of IFN-γ, IL-6, and MCP1, and/or wherein said patient is a patient that has developed an immune response to said immunotherapy, wherein said immune response is a side effect of said immunotherapy against said cancer, and/or wherein said method for treatment is a method for treatment in combination with allogeneic or autologous hematopoietic stem cell transplantation, and/or wherein said composition further comprises a pharmaceutically acceptable carrier, and/or wherein said composition is to be administered by a route other than oral administration, and/or wherein said cancer is a cancer other than chronic myeloid leukemia and acute lymphoblastic leukemia.

158. The method of claim 149, wherein the patient has a side effect associated with the immunotherapy.

159. The method of claim 158, wherein said immunotherapy is a CAR T immunotherapy and/or wherein said one or more side effects associated with immunotherapy are selected from the group consisting of:
I) cytokine release syndrome,
II) macrophage activation syndrome,
III) off-target toxicity,
IV) on-target/off-tumor recognition of normal and/or malignant cells,
V) rejection of immunotherapy cells,
VI) inadvertent activation of immunotherapy cells,
VII) tonic signaling and activation of immunotherapy cells, and/or
VIII) neurotoxicity,
IX) tumor lysis syndrome,
optionally wherein said side effect associated with immunotherapy is cytokine release syndrome and said cytokine release syndrome is characterized by elevated cytokine serum levels of one or more cytokines selected from the group consisting of GM-CSF, IFN-γ, IL-2, IL-4, IL-5, IL-6, IL-8, and IL-10, further optionally wherein said use is a use for causing a reduction of one or more of said elevated cytokine serum levels, and/or wherein said cytokine release syndrome is caused by said immunotherapy.

160. A method for modulating cells expressing a chimeric antigen receptor in a patient being treated for cancer using immunotherapy, comprising administering a tyrosine kinase inhibitor to the patient.

161. The method of claim 160, wherein said tyrosine kinase inhibitor is a dasatinib, saracatinib, bosutinib, nilotinib, or a PP1-inhibitor, and/or wherein the composition comprises a pharmaceutically acceptable carrier.

162. A composition, comprising:
I) an immune cell, and
II) a tyrosine kinase inhibitor.

163. The composition of claim 162, wherein said immune cell is an immune cell that expresses a chimeric antigen receptor, and/or wherein said tyrosine kinase inhibitor is a dasatinib, saracatinib, bosutinib, nilotinib, or a PP1-inhibitor, and/or wherein the composition comprises a pharmaceutically acceptable carrier.
